(19) United States
(12) Patent Application Publication
(10) Pub. No.: US 2025/0090597 A1
(43) Pub. Date: Mar. 20, 2025

(54) AIRWAY BASAL CELL ENGRAFTMENT METHODS

(71) Applicant: TRUSTEES OF BOSTON UNIVERSITY, Boston, MA (US)

(72) Inventors: Liang MA, Boston, MA (US); Darrell Kotton, Boston, MA (US); Finn Hawkins, Boston, MA (US)

(73) Assignee: TRUSTEES OF BOSTON UNIVERSITY, Boston, MA (US)

(21) Appl. No.: 18/792,994

(22) Filed: Aug. 2, 2024

Related U.S. Application Data
(60) Provisional application No. 63/530,370, filed on Aug. 2, 2023.

(51) Int. Cl. A61K 35/42; A61P 11/00; C12N 5/071

(52) U.S. Cl. CPC A61K 35/42; A61P 11/00; C12N 5/0688; C12N 2510/00

(57) ABSTRACT

The technology described herein is directed to engraftment methods for airway basal cells into the respiratory tract of a subject, following removal of epithelial cells of the respiratory tract. Also described herein are methods of treating a respiratory tract disease or a respiratory tract injury.

### CROSS-REFERENCE TO RELATED APPLICATIONS

This application claims benefit under 35 U.S.C. § 119(e) of U.S. Provisional Application No. 63/530,370 filed Aug. 2, 2023, the contents of which are incorporated herein by reference in their entirety.

### GOVERNMENT SUPPORT

This invention was made with government support under contract No. U01HL148692 and contract No. U24134763 which were awarded by the National Institutes of Health. The government has certain rights in the invention.

### TECHNICAL FIELD

The technology described herein relates to airway basal cell engraftment methods.

### BACKGROUND

Adult mammalian epithelial tissues are maintained throughout life through either the proliferation of common mature cells or the self-renewal and differentiation of tissue-resident stem cells. Engraftment of exogenous epithelial stem cells provides an opportunity to supplement or replenish endogenous reparative cells and has been attempted with success for ectodermally-derived epithelia. However, similar engraftment has been challenging to achieve for internal tissues. This in turn has prevented successful long-lived rescue of diseased internal epithelia, such as the respiratory airways (see e.g., Berical et al. (2019). Challenges Facing Airway Epithelial Cell-Based Therapy for Cystic Fibrosis. Front Pharmacol 10, 74). Reconstituting the tissue resident stem cell compartment of internal epithelial tissues has the advantage of resulting in durable and functional engraftment, since stem cells can undergo multipotent differentiation as well as self-renewal in vivo, if successfully engrafted.

The conducting airways of the lung presents a target for cell transplantation, since: a) the entire airway epithelium is in contact with the outside environment, providing access for exogenous cells; b) the resident stem cells of the pseudostratified respiratory epithelium, basal cells, are known and have been extensively characterized; and c) genetic diseases that affect the airway, such as cystic fibrosis or primary ciliary dyskinesia, remain significant sources of morbidity and mortality, which can be targeted by engraftment of gene-corrected airway stem cells. Yet the basal cell compartment of the airways remains difficult to target with cells or genes, as it is protected by both luminal cells that abut the airway surface and a highly effective innate immune system that have evolved to protect the airway epithelium from being penetrated by inhaled exogenous cells, pathogens, or particles.

While successful transplantation of exogenous cells, such as primary cells harvested from fetal or adult lungs, into the airway or alveolar epithelium of immunodeficient mouse recipients, durable reconstitution of the airway stem cell compartment has yet to be demonstrated; see e.g., Vaughan et al. (2015) Nature 517, 621-625; Nichane et al. (2017) Nat Methods 14, 1205-1212; Miller et al. (2018) Stem Cell Rep 10, 101-119; Kathiriya et al. (2020) Cell Stem Cell 26, 346-358.e4; Kathiriya et al. (2022) Nat Cell Biol 24, 10-23; Liao et al. (2022). Iscience 25, 104262; Louie et al. (2022) Cell Reports 39, 110662; Ghosh et al. (2016) Am J Resp Cell Mol 56, 1-10. Primary airway basal cells and their equivalents generated from pluripotent stem cells in vitro both represent cell types for transplantation, since they can be maintained and expanded in cell culture to generate the large numbers of cells with retained stem cell phenotype that is required for transplantation. Furthermore, both cell types can be stored as frozen archives or genetically manipulated, features that can facilitate future cell-based therapies.

There is thus great need for improved engraftment methods for airway basal cells.

### SUMMARY

The technology described herein is directed to engraftment methods for airway basal cells into the respiratory tract of a subject, following removal of epithelial cells of the respiratory tract. Also described herein are methods of treating a respiratory tract disease or a respiratory tract injury.

In one aspect, described herein is a method of engrafting airway basal cells into the respiratory tract of a subject in need thereof, the method comprising: (a) removing epithelial cells from the respiratory tract; and (b) administering airway basal cells to the respiratory tract.

In some embodiments of any of the aspects, the epithelium cells are removed from the respiratory tract a chemical agent or using bronchial thermoplasty.

In some embodiments of any of the aspects, the chemical agent comprises a detergent.

In some embodiments of any of the aspects, the chemical agent is selected from the group consisting of: polidocanol; 3-[(3-cholamidopropyl)dimethylammonio]-1-propane sulfonate (CHAPS); lucinactant, colfosceril palmitate, beractant, calefacient, and poractant alfa.

In some embodiments of any of the aspects, the chemical agent comprises polidocanol.

In some embodiments of any of the aspects, step (b) of administering the airway basal cells occurs 3 hours to 24 hours after step (a) of removing epithelium epithelial cells from of the respiratory tract.

In some embodiments of any of the aspects, the epithelial cells are removed in the trachea, at least one bronchus, and/or at least one bronchiole of the respiratory tract.

In some embodiments of any of the aspects, the airway basal cells are administered to the trachea, at least one bronchus, and/or at least one bronchiole of the respiratory tract.

In some embodiments of any of the aspects, the chemical agent and/or the airway basal cells are administered using a bronchoscope.

In some embodiments of any of the aspects, the airway basal cells are primary cells.

In some embodiments of any of the aspects, the airway basal cells are differentiated from pluripotent stem cells.

In some embodiments of any of the aspects, the airway basal cells express at least one marker selected from the group consisting of: Tumor Protein P63 (TRP63), keratin-5 (KRT5), and NK2 Homeobox 1 (NKX2-1).

In some embodiments of any of the aspects, the airway basal cells are autologous or allogeneic to the subject.

In some embodiments of any of the aspects, the airway basal cells are engineered to reduce or prevent expression of at least one major histocompatibility complex (MHC) polypeptide to reduce immunogenicity of the airway basal cells.

In some embodiments of any of the aspects, the subject is healthy.

In some embodiments of any of the aspects, the subject has a respiratory tract disease or a respiratory tract injury.

In some embodiments of any of the aspects, the respiratory tract disease is selected from the group consisting of: cystic fibrosis; primary ciliary dyskinesia; idiopathic pulmonary fibrosis (IPF); chronic obstructive pulmonary disease (COPD); lung cancer; asthma; chronic bronchitis; bronchiectasis; bronchiolitis obliterans syndrome; and chronic lung allograft disorder (CLAD).

In some embodiments of any of the aspects, the respiratory tract injury is associated with: inhalation of a chemical (e.g., an acid), smoke, fire, and/or a heated substance; a burn; radiation damage; chemotherapy damage; space travel; a respiratory infection; a birth defect; a lung cancer; and/or a surgery (e.g., tracheal resection, lung lobectomy).

In some embodiments of any of the aspects, the airway basal cells are engineered to express at least one functional polypeptide that is dysfunctional in a respiratory tract disease.

In some embodiments of any of the aspects, the airway basal cells are engineered to express functional Cystic Fibrosis Transmembrane Conductance Regulator (CFTR) polypeptide, and the subject has cystic fibrosis.

In some embodiments of any of the aspects, the airway basal cells are engineered to express functional Dynein Axonemal Heavy Chain 5 (DNAH5) polypeptide and/or functional Dynein Axonemal Intermediate Chain 1 (DNAI1) polypeptide, and the subject has primary ciliary dyskinesia.

In some embodiments of any of the aspects, the engrafted airway basal cells reconstitute the tissue-resident airway basal stem cell compartment in the respiratory tract of the subject.

In some embodiments of any of the aspects, the engrafted airway basal cells comprise at least 10% of the epithelium in the respiratory tract of the subject after the engraftment.

In some embodiments of any of the aspects, the engrafted airway basal cells persist for at least 2 years following the engraftment.

In some embodiments of any of the aspects, the engrafted airway basal cells exhibit self-renewal and/or multipotent differentiation capacity for at least 2 years following the engraftment.

In some embodiments of any of the aspects, the multipotent differentiation capacity comprises: basal cell, secretory cell, ciliated cell, ionocyte, neuroendocrine cell, tuft cell, and/or hillock cell airway epithelial lineages.

In some embodiments of any of the aspects, wherein the engrafted airway basal cells exhibit a transcriptome that is at least 80% identical to the transcriptome of endogenous airway epithelium cells.

In one aspect, described herein is a method of treating a respiratory tract disease, the method comprising: (a) removing epithelial cells from the respiratory tract of a subject in need thereof; and (b) administering an effective amount of airway basal cells to the respiratory tract; wherein the respiratory tract disease is selected from the group consisting of: cystic fibrosis; primary ciliary dyskinesia; idiopathic pulmonary fibrosis (IPF); chronic obstructive pulmonary disease (COPD); lung cancer; asthma; chronic bronchitis; bronchiectasis; bronchiolitis obliterans syndrome; and chronic lung allograft disorder (CLAD).

In one aspect, described herein is a method of treating a respiratory tract injury, the method comprising: (a) removing epithelial cells from the respiratory tract of a subject in need thereof; and (b) administering an effective amount of airway basal cells to the respiratory tract; wherein the respiratory tract injury is associated with: inhalation of a chemical (e.g., an acid), smoke, fire, and/or a heated substance; a burn; radiation damage; chemotherapy damage; space travel; a respiratory infection; a birth defect; a lung cancer; and/or a surgery (e.g., tracheal resection, lung lobectomy).

In one aspect, described herein is a kit comprising: (a) a chemical agent to remove epithelial cells from a respiratory tract; and (b) airway basal cells.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-1P: Generation and expansion in culture of murine iBC and primary BC culture in defined serum-free medium. FIG. 1A: Schematic of in vitro directed differentiation of murine pluripotent stem cells (PSCs) carrying an Nkx2-1^{mcherry} reporter into airway basal epithelial-like cells (iBC) followed by their expansion as monolayered epithelial spheres in serially passaged 3D serum-free, feeder free MATRIGEL cultures. RA=Retinoic acid; RI=ROCK Inhibitor Y27632; BCM=Basal Cell Medium. Representative D13 FACS plot is shown. FIG. 1B: Representative iBC culture (D52) at passage 1 (P1) after sorting NGFR+/Nkx2-1^{mCherry+} cells. Scale bars=200 μm. FIG. 1C: Representative FACS plots at different stages of the iBC directed differentiation protocol shown in FIG. 1A. D57 (P1 iBC) samples were analyzed by RT-qPCR in FIG. 1E, with right sorting gate representing NGFR+ samples and left sorting gate representing NGFR− samples. FIG. 1D: Quantification of percent of cells co-expressing Nkx2-1^{mCherry} and NGFR+ cells out of all live cells analyzed by FACS at P0 and P1, respectively. Bars indicate mean+/−SD. N=7 biological replicates. FIG. 1E: RT-qPCR analysis of P1 iBC culture. Statistical analysis performed by paired, two-tailed Student's t-test. Bars indicate mean+/−SD. N=3 biological replicates. FIG. 1F: Immunofluorescence confocal microscopy of paraffin embedded tissue sections of representative iBC epithelial spheres stained for NKX2-1 and KRT5 proteins. Scale bar=20 μm. Nuclei are counterstained with Hoechst33342. FIG. 1G: Quantification of iBC cell yield and growth kinetics over nine passages. Mean+/−SD are shown for three replicated differentiations separated at DO. FIG. 1H: Dimensionality reduction visualization by SPRING plots of iBC global transcriptomes profiled by scRNA-seq. Outgrowth of iBC cultures after six serial passages (P6; D129) were analyzed by Louvain Clustering (resolution=0.5), and cell clusters were annotated based on canonical markers. iBasal=PSC-derived basal-like cell; iSecretory=PSC-derived secretory-like cell. FIG. 1I: Selected canonical marker gene expression levels for each indicated marker shown overlaid on the SPRING plot from H. FIG. 1J: Heatmap of the top 20 differentially expressed genes (DEG) ranked by log fold change (Log FC) in each of the three cell clusters (C1-C3) annotated in FIG. 1H: iBasal, iSecretory, and Proliferating cluster. FIG. 1K: Schematic of generation and maintenance of primary BC cultures in basal cell medium (BCM). FIG. 1L: Representative live whole mount microscopy of P0 and P1 primary BC cultures. Scale bar=200 μm. FIG. 1M: Representative FACS plot of P1 primary BC culture. Samples were analyzed by RT-qPCR in Figure iN, with top sorting gate representing NGFR+ samples and bottom sorting gate representing NGFR-samples. FIG. 1N: RT-qPCR analysis of P1 primary BC cultures, sorted based on the gates shown in FIG. 1M. Statistical analysis performed by paired, two-tailed Student's t-test. Bars indicate mean+/−SD. N=3 biological replicates. FIG. 1O: Immunofluorescence confocal microscopy of paraffin embedded tissue sections of primary BC sphere cultures for KRT5, ACTUB, and SCGB1A1 proteins. Scale bar=20 μm. FIG. 1P: Quantification of cell yield and growth kinetics of primary BC over six serial passages in BCM. Bars indicate mean+/−SD. N=3 biological replicates.

FIG. 2A-2I: Transplantation of syngeneic murine primary BCs into immunocompetent recipient mice. FIG. 2A: Schematic summary of experimental approach for transplantation of cultured primary BC from UBC-GFP mice into polidocanol-injured immunocompetent syngeneic mice. FIG. 2B: Tracheal whole mount fluorescence microscopy and corresponding FACS of GFP (gated from all live cells harvested from the digest) to quantify donor-derived (GFP+) cells present in a representative recipient of primary BCs 69 days post-transplantation. Negative control tracheal whole mount fluorescence microscopy of a primary recipient exposed to sham (PBS) injury and cell delivery two weeks post-transplantation is shown. FIG. 2C: Immunofluorescence confocal microscopy for GFP, ACTUB, SCGBlA1, KRT5, and NKX2-1 proteins in a representative primary BC recipient trachea six months post-transplantation. White arrows indicate GFP+(donor-derived) cells co-expressing the indicated canonical airway epithelial markers. Upper panels show all indicated fluorescence channels, and lower panels (“w/o GFP”) have the GFP channel removed. Nuclei are counterstained with Hoechst33342. Scale bar=10 μm. FIG. 2D: Schematic for cell capture and scRNA-Seq analysis of endogenous (GFP−) and donor-derived (GFP+) tracheal epithelial cells from recipients of primary BCs. FIG. 2E: SPRING visualization of single cell transcriptomic profiles from primary BC recipient trachea; 69 days post-transplantation. Cells are designated by sample origin. Separated view of donor-derived and endogenous cell clusters are shown (see e.g., FIG. 10A). FIG. 2F: Louvain clustering and annotation of cells from the SPRING shown in FIG. 2G (resolution=0.2). FIG. 2G: Lineage frequency of donor-derived vs endogenous tracheal epithelial cells. FIG. 2H: Dot plot comparing canonical gene expression levels and frequencies between donor-derived cell lineages and their endogenous counterparts from FIG. 2G. FIG. 2I: Violin plot comparing selected canonical gene expression between donor-derived lineages and their endogenous counterparts. Statistical analysis is done with Wilcoxon signed-rank test with adjusted-p values shown only for significantly different expression levels.

FIG. 3A-3H: Transplantation of murine iBCs into syngeneic immunocompetent recipient mice. FIG. 3A: Schematic summary of experimental approach for transplantation of cultured iBC, tagged with lentiviral GFP, into polidocanol-injured immunocompetent syngeneic mice. FIG. 3B: Representative whole mount live fluorescence microscopy and FACS of donor cell population, quantifying NGFR, GFP, Nkx2-1^{mcherry} expression and forward scatter (FSC). Scale bar=200 μm. FIG. 3C: Whole mount fluorescence and corresponding FACS quantification (gated from all live cells harvested from the digest) of iBC recipient 56-days and 192-days post-transplantation. FIG. 3D: Tracheal whole mount fluorescence microscopy and FACS of recipients of GFP+iBCs, shown at various timepoints (three weeks to one year) post-transplantation. FIG. 3E: Quantification of primary BC (N=10) and iBC (N=19) transplantation efficiency, calculated by GFP+% area over total recipient trachea area in epifluorescence image. Comparison between epifluorescence imaging quantification and corresponding FACS transplantation efficiency quantification is shown in FIG. 11C. FIG. 3F: Immunofluorescence confocal microscopy for GFP, ACTUB, SCGB1A1, KRT5, and NKX2-1 in iBC recipient trachea 1-year post-transplantation. White arrows indicate GFP+ donor-derived cells co-expressing indicated canonical airway epithelial markers. Lower panels are with GFP channel removed. Scale bar=10 μm. FIG. 3G: Tracheal GFP fluorescence in an unfixed, unstained frozen tissue section and H&E staining of the immediately adjacent tissue section of a recipient of GFP+iBCs, seven weeks post-transplantation. FIG. 3H: In vivo bioluminescence imaging of four recipients and an untransplanted control followed for six months after receiving transplanted iBCs tagged with GFP-luciferase-expressing lentivirus. Transplantation efficiency of each recipient (quantified by FACS in FIG. 13) is shown.

FIG. 4A-4G: Characterization of iBC donor-derived vs endogenous epithelial cells in recipient tracheas by single cell transcriptomic profiling. FIG. 4A: Schematic of experimental approach for cell capture and scRNA-Seq profiling of epithelial cells from tracheas of three recipients of GFP+iBCs at 40-days, 56-days, and 192-days post-transplantation. One “no transplant” control animal (no transplant, no injury) was included in the experiment as shown in FIG. 4B. FIG. 4B: SPRING visualization of single cell transcriptomic profiles of tracheal cells from recipients of iBC transplants as shown in FIG. 4A. Cells are designated by sample origin. FIG. 4C: SPRING visualization of single cell transcriptomic profiles of tracheal cells from recipients of iBC transplants as shown in FIG. 4A. Cells are designate by annotated Louvain Clustering (resolution=0.2). FIG. 4D: Lineage frequency of donor derived vs. endogenous tracheal cells for each recipient from FIG. 4B. FIG. 4E: Selected canonical gene expression shown as green overlays on SPRING plot from FIG. 4B. FIG. 4F: Violin plots comparing expression levels of selected canonical marker genes between donor-derived lineages and their endogenous counterparts from panel FIG. 4B. Statistical analysis is done with Wilcoxon signed-rank test with adjusted-p values shown only for significantly different expression levels. FIG. 4G: Dot plot comparing canonical gene expression between donor-derived lineages and their endogenous counterparts from panel FIG. 4B.

FIG. 5A-5G: In vivo stem cell self-renewal and differentiated cell function of engrafted murine iBC-derived cells. FIG. 5A: Representative screen capture of live GFP fluorescence by high content video confocal microscopy imaging; indicating GFP+(donor-derived) and GFP− (endogenous) multiciliated cells (cilia labeled with wheat germ agglutinin in white); graph shows quantification of cilia beating frequency (events per unit time) of donor-derived and endogenous cells. Scale bar=5 μm. FIG. 5B: Whole mount immunofluorescence microscopy of GFP and acetylated tubulin (ACTUB) proteins in recipient trachea 310 days after transplantation of GFP+iBCs, indicating donor-derived (GFP+) multiciliated cells; graph shows quantification of cilia length in donor-derived and endogenous multiciliated cells. N=44 cells per condition. Scale bar=20 μm. FIG. 5C: Schematic summary of experimental approach for assessing response of donor iBC-derived engrafted cells to in vivo re-injury from repeated polidocanol exposure. FIG. 5D: Immunofluorescence confocal microscopy of representative tracheal tissue section for GFP, KRT5, EdU, and Ki67 in iBC recipient trachea 1 week post-second polidocanol injury and corresponding quantification. White arrows indicate donor-derived (GFP+) cells that co-label with EdU. Scale bar=10 μm. N=2 animals (411 donor-derived KRT5+ cells and 262 endogenous KRT5+ cells). Statistical analysis performed by unpaired, two-tailed Student's t-test. FIG. 5E: Schematic summary of experimental approach for serial transplantations of GFP+ tagged iBCs into secondary recipients to measure self-renewal capacity. FIG. 5F: Tracheal whole mount fluorescence microscopy and corresponding FACS analysis (gated from all live cells harvested from the digest) of secondary recipients of GFP+iBCs, characterizing seven generations of serial iBC transplants. 7^{th} generation (7°) recipient trachea is also shown in analysis in FIG. 13A. FIG. 5G: Immunofluorescence confocal microscopy for GFP, ACTUB, SCGB1A1, KRT5, NKX2-1 in a 5^{th} generation (5°) iBC recipient trachea. White arrows indicate GFP+ donor-derived cells co-expressing indicated canonical airway epithelial markers. Lower panel shows GFP channel removed. Scale bar=10 μm.

FIG. 6A-6E: Characterization of iBC donor-derived vs endogenous epithelial cells in 5° generation recipient tracheas by single cell transcriptomic profiling. FIG. 6A: Whole mount epifluorescence imaging and corresponding FACS analysis of 5° generation recipient animals profiled by scRNA-Seq. FIG. 6B: SPRING visualization of scRNA-Seq of 5^{th} generation recipients, designated by sample or by Louvain Clustering (resolution=0.2). FIG. 6C: Selected canonical gene expression shown as gray overlays on SPRING plot from FIG. 6B. FIG. 6D: SPRING visualization of merged scRNA-Seq dataset of 1° recipients (see e.g., FIG. 4A-4G) and 5° generation recipients without harmonization. Selected samples are highlighted in the lower small panels. FIG. 6E: SPRING visualization of merged scRNA-Seq dataset of 1° generation recipients (see e.g., FIG. 4A-4G) and 5^{th} generation recipients without harmonization, colored by Louvain Clustering (resolution=0.2).

FIG. 7A-7H: Characterization of engrafted human primary BCs or iBCs in injured NSG mouse recipient tracheas. FIG. 7A: Schematic of transplantation of human primary BCs (human bronchial epithelial cells; HBECs) or iBCs into polidocanol-injured NSG mouse trachea. Note lentiviral GFP or tdTomato tagging of cultured cells prior to transplantation. FIG. 7B: Whole mount fluorescence and corresponding FACS quantification (gated from all live cells harvested from the digest) of derivatives of either GFP+ human primary BCs or tdTomato+ human iBCs in mouse recipient tracheas six weeks post-transplantation. FIG. 7C: Immunofluorescence confocal microscopy for GFP, tdTomato (RFP), ACTUB, SCGB1A1, KRT5, and NKX2-1 in mouse recipient tracheas six weeks post-transplantation of human cells. White arrows indicate GFP+ or RFP+ human donor-derived cells co-expressing canonical airway epithelial markers. Arrows with asterisks indicate human donor-derived cells with goblet cell morphology. Lower panels show staining with the GFP or RFP channel removed as indicated. Scale bar=10 μm. FIG. 7D: Single cell transcriptomic profiling of human donor-derived cells in recipient mouse tracheas; SPRING visualization representing scRNA-Seq of human cells in mouse recipient tracheas profiled six weeks post-transplantation. Cells are designated by sample origin. FIG. 7E: Louvain clustering and annotations of human cells in each recipient from FIG. 7D (resolution=0.1). FIG. 7F: Selected canonical marker gene expression (gray) overlayed on SPRING plots from FIG. 7D-7E. FIG. 7G: Violin plots comparing selected canonical gene expression levels between engrafted human primary BC-derived lineages vs human iBC-derived lineages. FIG. 7H: Heatmap of the top 532 most variable genes (filtered by Log FC>0.5 and FDR-adjusted p<0.05; across Louvain clusters from E). Stressed population (C5) is excluded from this heatmap analysis.

FIG. 8A-8E: Characterization of murine iBC and primary BC cultures (see e.g., FIG. 1A-1P). FIG. 8A: Representative G-banding analysis indicating karyotypically normal undifferentiated murine ESCs and differentiated iBCs. FIG. 8B: Representative images of iBC cultures at P1, P4, and P9. Scale bars, 200 μm. FIG. 8C: RT-qPCR analysis of iBC and primary BC cultures. Bars indicate mean+/−SD. n=3 biological replicates. For comparisons with three or more samples, one-way ANOVA was used. For comparisons with only two samples, paired two-tail Student's t-test was used. FIG. 8D: Representative image and corresponding FACS analysis of iBC culture two passages (P2; D81) after thawing from cryopreservation. Scale bars, 200 μm. FIG. 8E: RT-qPCR analysis of iBC P2 after thawing from cryopreservation. Bars indicate mean+/−SD. n=3 biological replicates.

FIG. 9A-9F: Post-injury mouse tracheal histological time series assessment and Syngeneic transplantation of murine primary BC into immunocompetent recipient mice (see e.g., FIG. 2A-2I). FIG. 9A: Representative H&E staining of mouse tracheas 3 hours, 5 hours, 7 hours, and 12 hours post-polidocanol injury. At 7 hrs post-injury, black arrow indicates flattened cells covering the basement membrane. At 12 hrs post-injury, arrow indicates clusters of epithelial cells. Scale bars, 50 μm. FIG. 9B: Tracheal whole mount fluorescence microscopy and FACS of recipients of GFP+ primary BCs, shown at various timepoints (1 month, 6 months, or 15 months, n=9) post-transplantation. FIG. 9C: FACS analysis of primary BC recipient at 6-months or 15-months post-transplantation, demonstrating persistence of donor-derived cells in recipient tracheas. FIG. 9D: SPRING visualization of scRNA-Seq of primary BC recipient, separated by sample origin (also shown in FIG. 2E). FIG. 9E: Selected canonical marker gene expression levels (in gray) overlayed on combined SPRING plots from FIG. 9D. FIG. 9F: Spearman correlation coefficients of single cell transcriptomic profiles of donor-derived and endogenous cells in recipients of primary mouse BCs (69 days post transplantation) based on the Top 3000 most variable genes.

FIG. 10A-10D: Syngeneic transplantation of murine iBC into immunocompetent recipient mice (see e.g., FIG. 3A-3H). FIG. 10A: Representative G-banding analysis indicating karyotypically normal donor iBCs after lentiviral transduction, cryopreservation, and thawing prior to transplantation. FIG. 10B: Immunofluorescence confocal microscopy for GFP, KRT5, and E-Cadherin in iBC recipient trachea 1-year post-transplantation. White arrows indicate GFP+ cells co-expressing KRT5. Lower panels have the GFP channel removed. Scale bars, 10 μm. FIG. 10C: Left: representative distal lung immunofluorescence microscopy of iBC recipient. Right: in rare cases, GFP+ signal was observed in the intrapulmonary large and small (distal) airways. Scale bars, 200 μm. FIG. 10D: Immunofluorescence confocal microscopy for GFP, ACTUB, SCGB1A1, KRT5, and NKX2-1 in iBC recipient intrapulmonary airway. White arrows indicate GFP+ cells co-expressing canonical airway epithelial markers. Lower panels have the GFP channel removed. Scale bars, 20 μm.

FIG. 11A-11D: Dynamic changes post-murine iBC transplantation (see e.g., FIG. 3A-3H). FIG. 11A: Whole mount fluorescence of iBC recipients 1, 3, 5, or 7 days post-transplantation. FIG. 11B: Immunofluorescence confocal microscopy for GFP, ACTUB, SCGB1A1, KRT5, NKX2-1 in iBC recipient tracheas at 1 day, 3 days, 5 days, or 7 days post-transplantation. White arrows indicate GFP+ cells co-expressing canonical airway epithelial markers. Lower panels have the GFP channel removed. Scale bars, 10 μm. FIG. 11C: Whole mount tracheal fluorescence and corresponding FACS analysis (gated from all live cells harvested from the digest) of recipients of iBCs labeled with GFP-Luciferase expressing lentivirus as shown in FIG. 3H. Transplantation efficiencies measured by GFP+% area over total trachea area (marked by dotted line) are shown below each recipient trachea image. FIG. 11D: Corresponding FACS plots for tracheas shown in FIG. 11C.

FIG. 12A-12H: Single cell transcriptomic profiling of murine iBC recipient tracheas (see e.g., FIG. 4A-4G). FIG. 12A: SPRING Visualization of iBC recipient tracheas profiled by scRNA-Seq. Cells are designated by Louvain clustering (resolution=0.2) and annotated as in FIG. 4C. FIG. 12B: Expression of rare cell canonical gene markers (green) overlayed on SPRING plots from FIG. 12A. FIG. 12C: Expression of selected genes associated with H2-K1+Scgb1a1^{low} airway progenitor cells by scRNA-Seq. FIG. 12D: Gene set enrichment analysis of top differentially expressed gene (1406 total) in the Ccl9^{high} cluster. NES=normalized enrichment score. FIG. 12E: Heatmap of Top 20 differentially expressed genes (DEG) between each of the indicated iBC-derived lineages and their endogenous counterparts. FIG. 12F: Violin plots comparing club cell proximal-distal patterning markers between endogenous and donor-derived secretory cells. Statistical analysis done with Wilcoxon signed-rank test, with adjusted p values shown only for significantly different expression levels. FIG. 12G: Immunofluorescence confocal microscopy for GFP and SCGB1A1 or SCGB3A2 in iBC recipient tracheas. Arrows with asterisks indicate GFP+ cells co-expressing SCGB1A1 or SCGB3A2. Arrows without asterisks indicate GFP− (endogenous) cells expressing SCGB1A1 or SCGB3A2. Scale bar, 10 μm. FIG. 12H: Quantification of SCGB1A1+ or SCGB3A2+ cells among donor-derived (GFP+) or endogenous cells. Statistical analysis performed by unpaired, two-tailed Student's t-test (n=3 animals, 1165 donor-derived cells, 934 endogenous cells).

FIG. 13A-13B: Dynamic changes post-murine iBC serial transplantation into 7^{th} generation (7°) recipients (see e.g., FIG. 5A-5G). FIG. 13A: Whole mount fluorescence of iBC recipients 1 day, 3 days, 5 days, or 7 days post-transplantation. FIG. 13B: Immunofluorescence confocal microscopy for GFP, SCGB1A1, and KRT5 in 7° iBC recipient tracheas at 1 day, 3 days, 5 days, or 7 days post-transplantation. White arrows indicate GFP+ cells co-expressing canonical airway epithelial markers. Lower panels have the GFP channel removed. Scale bars, 10 μm.

FIG. 14A-14H: Characterization of human or mouse cells post-transplantation into injured NSG recipients (see e.g., FIG. 7A-7H). FIG. 14A: Whole mount fluorescence of iBC (BU3-NGPT and KOLF2) recipient tracheas (BU3 NGPT n=11; KOLF2 n=5) at various harvest timepoints (4 weeks, 6 weeks, or 29 weeks). FIG. 14B: Quantification of human iBC (BU3-NGPT indicated by black dots and KOLF2 indicated by gray dots) transplantation efficiency, shown as RFP+% area over total recipient trachea area. FIG. 14C: Whole mount fluorescence of NSG recipient tracheas that received murine iBC transplantation at various harvest timepoints (2 weeks or 18 months). FIG. 14D: Quantification of murine iBC into NSG recipient transplantation efficiency, shown as GFP+% area over total recipient trachea area. FIG. 14E: FACS and Immunofluorescence confocal microscopy for GFP, ACTUB, SCGB1A1, Ki67, and KRT5 in NSG recipient that received murine iBC 18 months post-transplantation. White arrows indicate GFP+ cells co-expressing canonical airway epithelial markers. Scale bar, 10 μm. FIG. 14F: Immunofluorescence confocal microscopy for RFP (iBC) or GFP (HBEC) and NKX2-1 in the intrapulmonary airway of NSG recipients. White arrows indicate RFP+ or GFP+ cells. Scale bar, 50 μm. FIG. 14G: Immunofluorescence confocal microscopy for GFP, KI67, KRT5, SCGB1A1, ACTUB, and MUC5B in the intrapulmonary airway of NSG recipient. Scale bar, 10 μm. FIG. 14H: SPRING visualization of scRNA-Seq of human cell transplantation recipients, designated by Louvain clustering (resolution=0.1) or sample of origin as in FIG. 7A-7H. Mitochondrial gene percentage of human cells profiled by scRNA-seq six weeks after transplantation into mouse recipient tracheas is also shown.

FIG. 15: Murine iBC-derived ciliated cells post-transplantation (see e.g., FIG. 5A-5G). Representative screenshots from live whole-mount video microscopy of iBC-recipient trachea. Donor-derived cells are shown. Cilia are shown in white.

### DETAILED DESCRIPTION

Embodiments of the technology described herein are directed to engraftment methods for airway basal cells into the respiratory tract of a subject, following removing epithelial cells from the respiratory tract of the subject. Also described herein are methods of treating a respiratory tract disease or a respiratory tract injury.

Herein, the airway stem cell compartment of mice was reconstituted by engraftment of mouse or human primary or PSC-derived basal stem cells (primary BC or iBC). After airway epithelial damage with polidocanol, which removed epithelial cells from the respiratory tract, transplantation of murine BCs or iBCs resulted in engrafted basal cells with self-renewal and multilineage differentiation capacity, the defining features of airway basal stem cells. iBCs engrafted in recipient syngeneic mice can persist for more than two years, and can be serially transplanted through seven generations of secondary mouse recipients. Furthermore, in vivo engraftment of injured NSG mouse recipient airway epithelium was demonstrated with human primary BCs and human iBCs. Collectively, these results establish primary basal cell and PSC-derived basal cell transplantation as a method for in vivo reconstitution of an internal epithelial tissue's resident stem cell compartment and can be used as an autologous cell-replacement therapy for patients with airway diseases.

### Removal of Respiratory Tract Epithelial Cells

In multiple aspects, the methods and compositions described herein relate to injuring or inducing damage to epithelium of the respiratory tract of a subject, including by removing epithelial cells from the respiratory tract. Such removal of epithelial cells, also referred to as denudation, can expose the basement membrane underlaying the epithelial cells and allow for successful engraftment of airway basal cells. In some embodiments, the epithelial cells of the respiratory tract are removed using a chemical agent. In some embodiments, the epithelial cells of the respiratory tract are removed using bronchial thermoplasty. In some embodiments, the epithelial cells of the respiratory tract are removed using a chemical agent and bronchial thermoplasty.

In some embodiments, the chemical agent comprises a detergent. In some embodiments, the chemical agent comprises a surfactant. In some embodiments, the chemical agent comprises a clinically approved detergent or surfactant. As used herein, the term “detergent” refers to a surfactant or a mixture of surfactants, including any anionic, cationic, or nonionic detergent capable of inducing lysis of or otherwise damaging epithelial cells. Detergents and surfactants are amphiphile molecules that can interact with and disturb cell membranes by modifying the lipid organization, leading to cell lysis and death. Detergents or surfactants can thus result in the removal of epithelial cells at or near the site of delivery of such detergents or surfactants.

Non-limiting examples of detergents include polidocanol, 3-[(3-cholamidopropyl)dimethylammonio]-1-propane sulfonate (CHAPS), guanidinium thiocyanate, Sodium dodecyl sulfate (SDS), Sodium lauryl sulfate, NP40 (nonyl phenoxypolyethoxylethanol), TRITON X-100 (octyl phenol ethoxylate), TWEEN (polysorbate), Sodium cholate, Sodium deoxycholate, Benzethonium chloride, Cetyltrimethylammonium bromide (CTAB), Hexadecyltrimethylammonium bromide, or N,NDimethyldecylamine-N-oxide, or any combinations thereof. In some embodiments, the detergent comprises polidocanol and/or CHAPS. Non-limiting examples of surfactants which are approved for clinical pulmonary use and can used as the chemical agent include: lucinactant, colfosceril palmitate, beractant, calefacient, or poractant alfa.

In some embodiments, the chemical agent comprises polidocanol (C₃₀H₆₂O₁₀; see e.g., Formula I below). In some embodiments, the chemical agent comprises about 2% polidocanol. In some embodiments, the chemical agent comprises about 1.75% polidocanol. In some embodiments, the chemical agent comprises about 1.5% polidocanol. In some embodiments, the chemical agent comprises about 1% polidocanol. In some embodiments, the chemical agent comprises about 0.5% polidocanol.

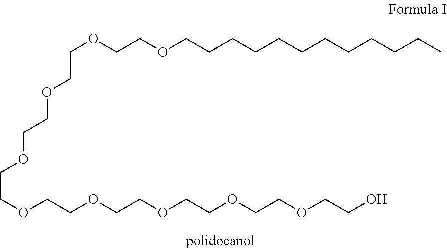 [SMILES: CCCCCCCCCCCCOCCOCCOCCOCCOCCOCCOCCOCCOCCO]

In some embodiments, the chemical agent comprises 0.5%-2%, 0.5%-1.75%, 0.5%-1.5%, 0.5%-1%, 0.5%-0.75%, 0.75%-2%, 0.75%-1.75%, 0.75%-1.5%, 0.75%-1%, 1%-2%, 1%-1.75%, 1%-1.5%, 1.5%-2%, 1.5%-1.75%, or 1.75%-2% of a detergent (e.g., polidocanol). In some embodiments, the chemical agent comprises at least 0.5%, at least 0.55%, at least 0.6%, at least 0.65%, at least 0.7%, at least 0.75%, at least 0.8%, at least 0.85%, at least 0.9%, at least 0.95%, at least 1%, at least 1.05%, at least 1.1%, at least 1.15%, at least 1.2%, at least 1.25%, at least 1.3%, at least 1.35%, at least 1.4%, at least 1.45%, at least 1.5%, at least 1.55%, at least 1.6%, at least 1.65%, at least 1.7%, at least 1.75%, at least 1.8%, at least 1.85%, at least 1.9%, at least 1.95%, or 2% of a detergent (e.g., polidocanol).

In some embodiments, the chemical agent comprises at most 0.5%, at most 0.55%, at most 0.6%, at most 0.65%, at most 0.7%, at most 0.75%, at most 0.8%, at most 0.85%, at most 0.9%, at most 0.95%, at most 1%, at most 1.05%, at most 1.1%, at most 1.15%, at most 1.2%, at most 1.25%, at most 1.3%, at most 1.35%, at most 1.4%, at most 1.45%, at most 1.5%, at most 1.55%, at most 1.6%, at most 1.65%, at most 1.7%, at most 1.75%, at most 1.8%, at most 1.85%, at most 1.9%, at most 1.95%, or at most 2% of a detergent (e.g., polidocanol).

In some embodiments, the chemical agent comprises about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, or about 2% of a detergent (e.g., polidocanol).

In some embodiments, the epithelial cells of the respiratory tract are removed using bronchial thermoplasty. Bronchial thermoplasty uses radiofrequency energy to heat the airway wall. A catheter inside a bronchoscope can be used to deliver thermal energy into the airways. As a non-limiting example, the catheter can deliver a series of 10-second temperature-controlled bursts of radio frequency energy, which heat the lining of the lungs to about 65 degrees Celsius. Such heating can result in the cell lysis and death, and thus removal of epithelial cells at or near the site of the bronchial thermoplasty.

In some embodiments, the bronchial thermoplasty heats the epithelial of the respiratory tract to at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C., or more.

In some embodiments, the bronchial thermoplasty heats the epithelial of the respiratory tract to at most 45° C., at most 46° C., at most 47° C., at most 48° C., at most 49° C., at most 50° C., at most 51° C., at most 52° C., at most 53° C., at most 54° C., at most 55° C., at most 56° C., at most 57° C., at most 58° C., at most 59° C., at most 60° C., at most 61° C., at most 62° C., at most 63° C., at most 64° C., at most 65° C., at most 66° C., at most 67° C., at most 68° C., at most 69° C., or at most 70° C.

In some embodiments, the bronchial thermoplasty heats the epithelial of the respiratory tract to about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., 45° C. to 50° C., 50° C. to 55° C., 55° C. to 60° C., 60° C. to 65° C., or 65° C. to 70° C.

In some embodiments, the bronchial thermoplasty heats the epithelial of the respiratory tract for a total of at least 10 seconds, at least 20 seconds, at least 30 seconds, at least 40 seconds, at least 50 seconds, at least 60 seconds, at least 70 seconds, at least 80 seconds, at least 90 seconds, at least 100 seconds, at least 110 seconds, at least 120 seconds, at least 3 minutes, at least 4 minutes, at least 5 minutes, at least 6 minutes, at least 7 minutes, at least 8 minutes, at least 9 minutes, at least 10 minutes, or more.

In some embodiments, epithelium cells are removed from the trachea, at least one bronchus, and/or at least one bronchiole of the respiratory tract. In some embodiments, epithelium cells are removed from the trachea. In some embodiments, epithelium cells are removed from at least one bronchus. In some embodiments, epithelium cells are removed from at least one bronchiole. In some embodiments, epithelium cells are removed from the trachea and at least one bronchus. In some embodiments, epithelium cells are removed from the trachea and at least one bronchiole. In some embodiments, epithelium cells are removed from at least one bronchus and at least one bronchiole. In some embodiments, epithelium cells are removed from the trachea, at least one bronchus, and at least one bronchiole. In some embodiments, epithelium cells are removed from at least one alveolus.

In some embodiments, the removal of epithelial cells results in an airway epithelium loss (e.g., denudation) of at least 10%, at least 15%, at least 20%, at least 25%, at least 30%, at least 35%, at least 40%, at least 45%, at least 50%, at least 55%, or at least 60%. In some embodiments, the removal of epithelial cells results in an airway epithelium loss (e.g., denudation) of at most 10%, at most 15%, at most 20%, at most 25%, at most 30%, at most 35%, at most 40%, at most 45%, at most 50%, at most 55%, or at most 60%. In some embodiments, the removal of epithelial cells results in an airway epithelium loss (e.g., denudation) of about 10%, about 15%, about 20%, about 25%, about 30%, about 35%, about 40%, about 45%, about 50%, about 55%, or about 60%.

### Airway Basal Cells

In multiple aspects, the methods and compositions described herein relate to administration of airway basal cells. Airway basal cells are found deep in the respiratory epithelium, attached to, and lining the basement membrane. As such, methods for successful engraftment of airway basal cells can include removal of epithelium cells from the respiratory tract, e.g., to expose the basement membrane for attachment by the transplanted airway basal cells.

Basal cells are the stem cells or progenitors of the airway epithelium and can differentiate to replenish all of the epithelial cells including the ciliated cells and/or secretory goblet cells. Basal cells are cuboidal with a large nucleus, few organelles, and scattered microvilli. The numbers of basal cells are highest in the large airways and become increasingly decreased in the smaller airways. Their percentage in the trachea is about 34%, in the large bronchi about 27%, and about 10% in the larger of the bronchioles.

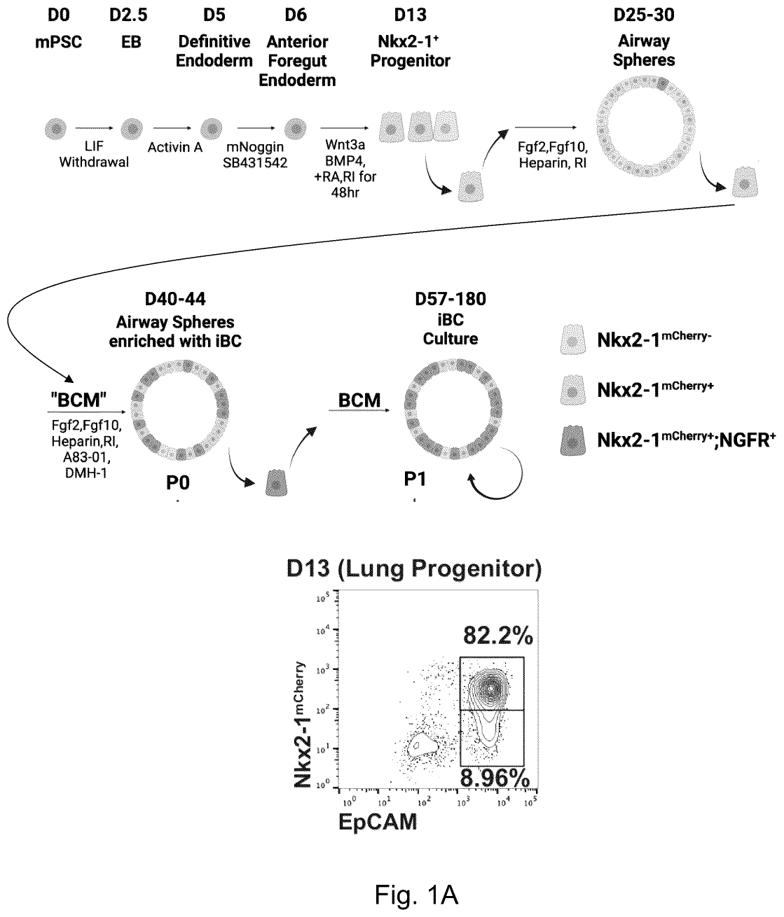

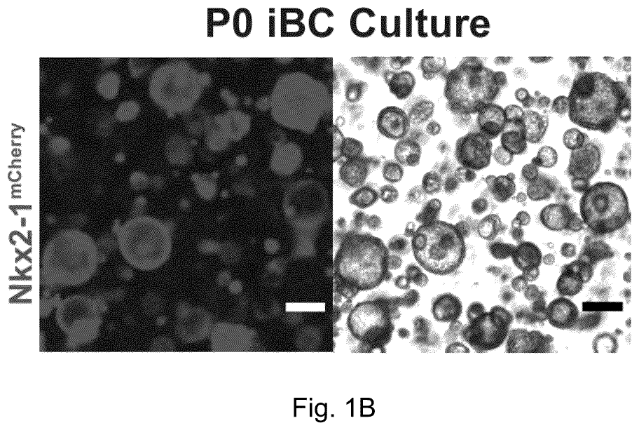

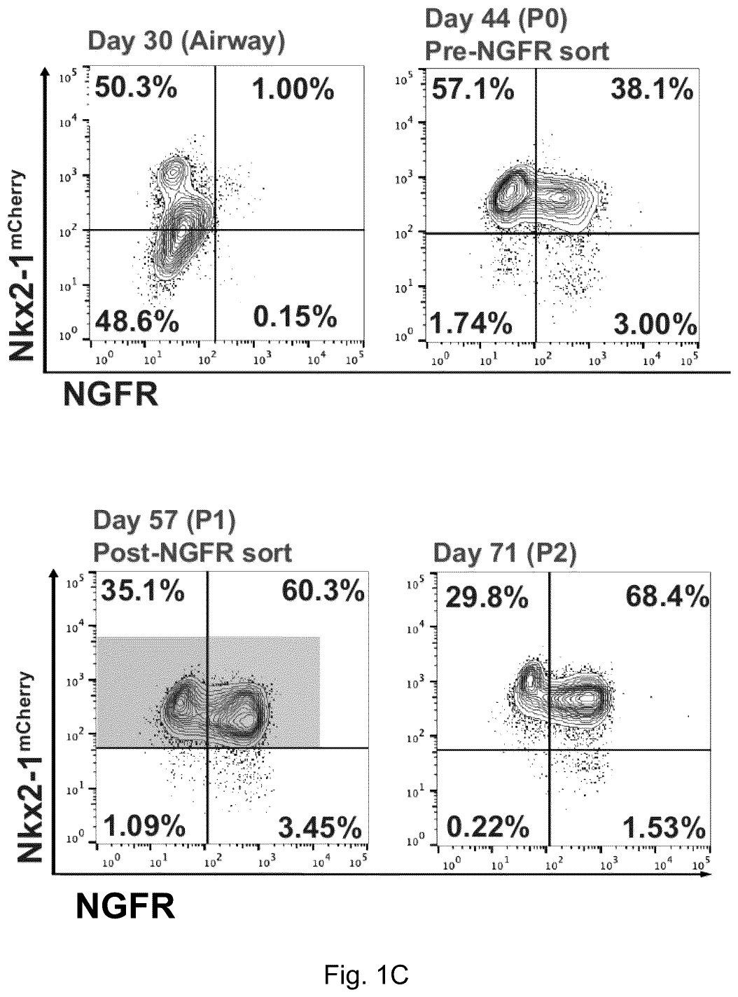

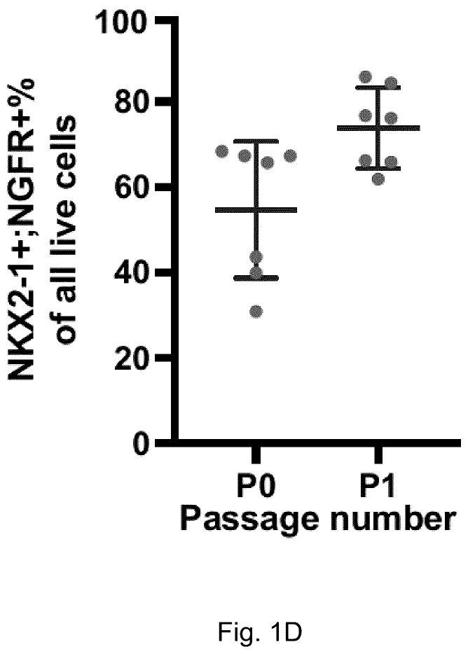

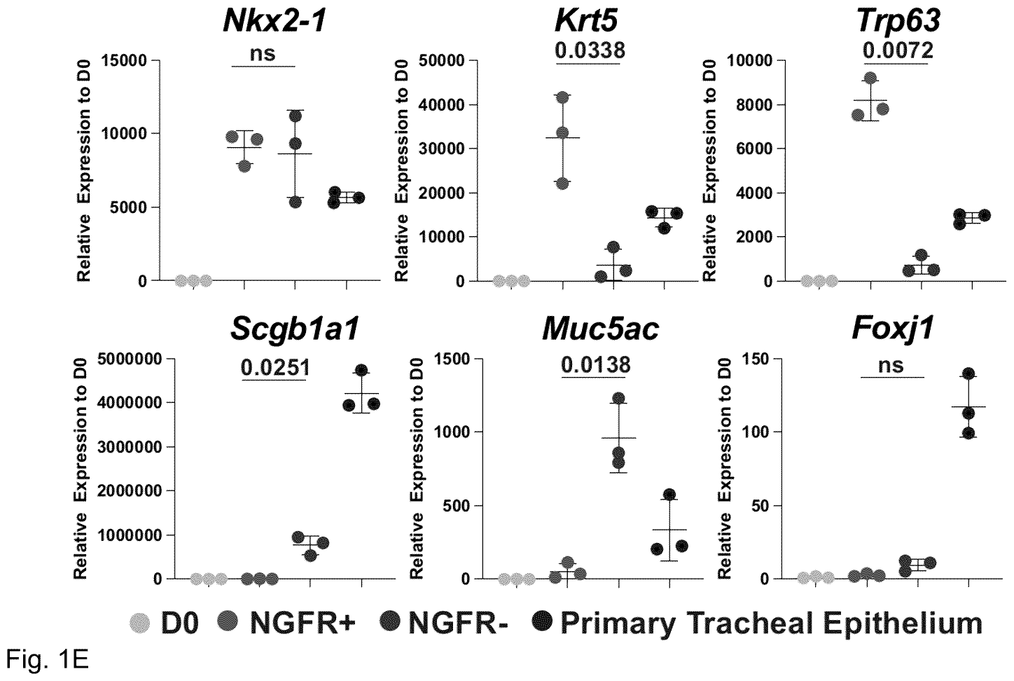

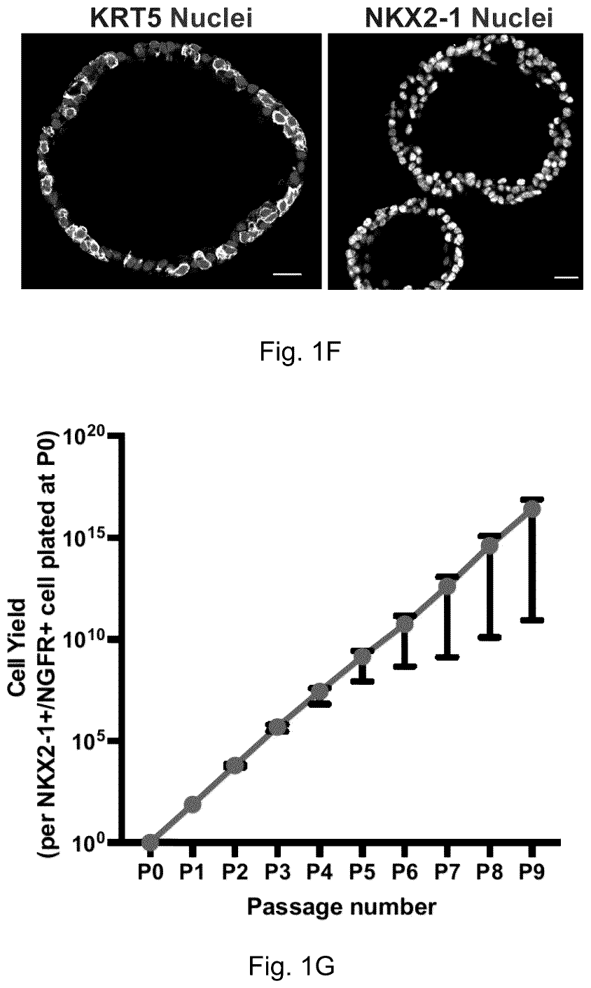

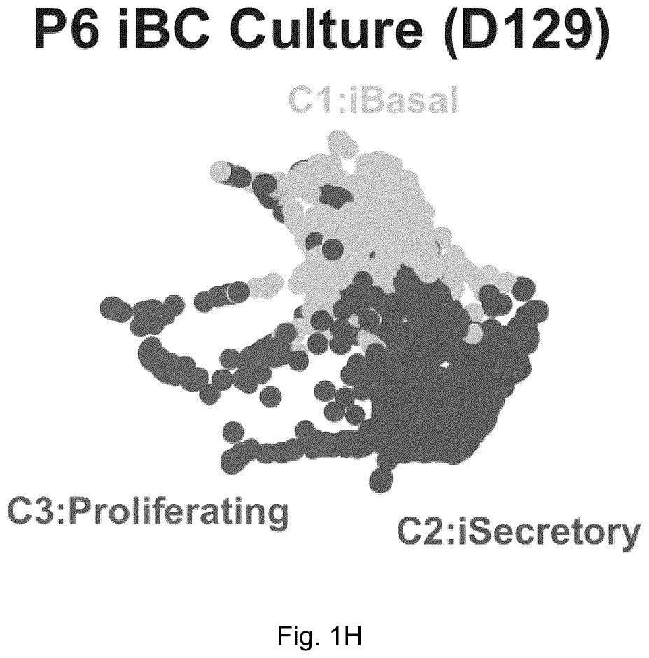

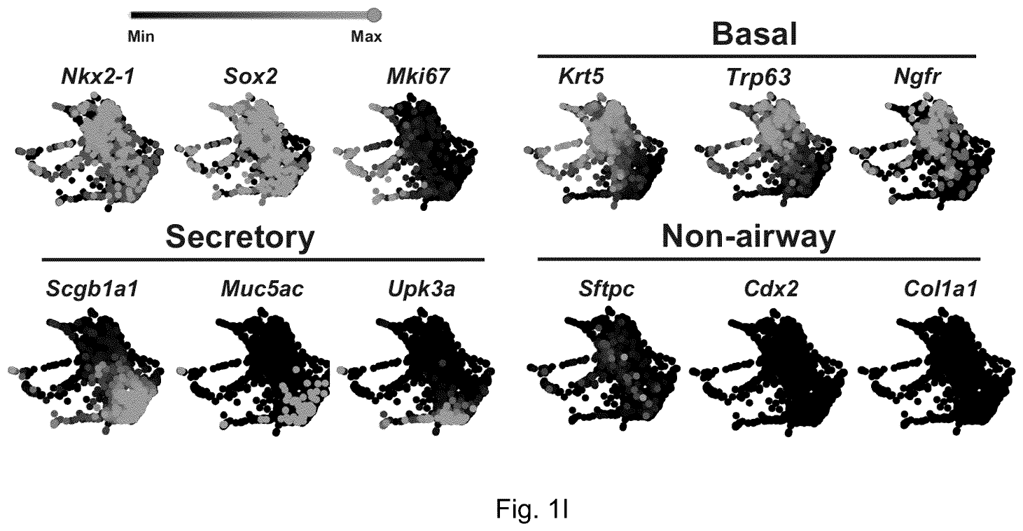

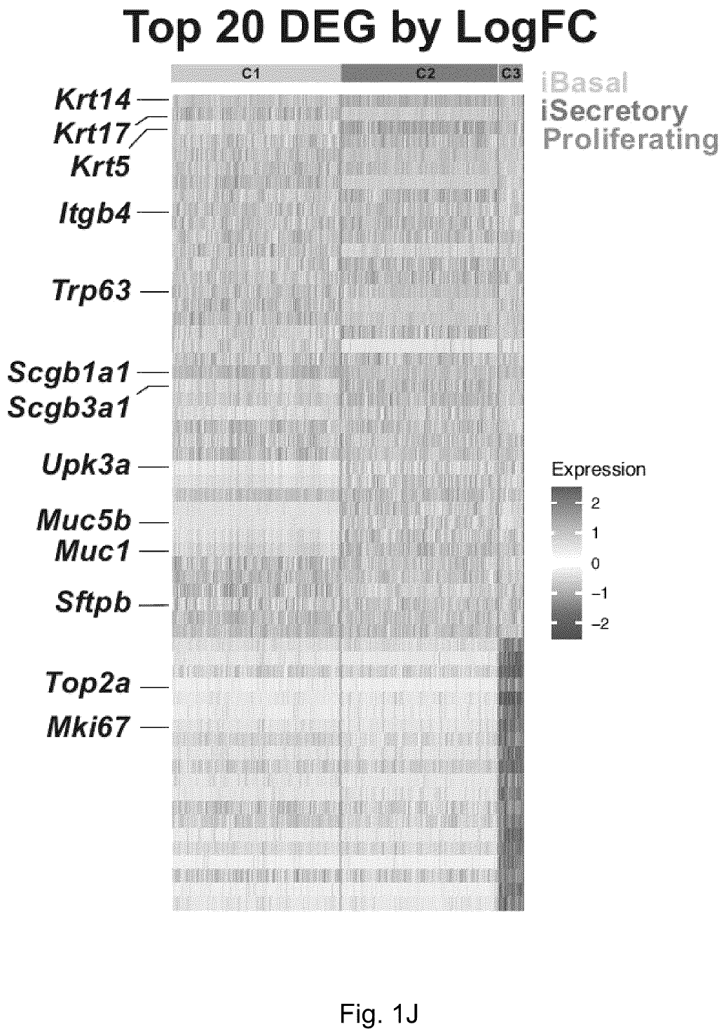

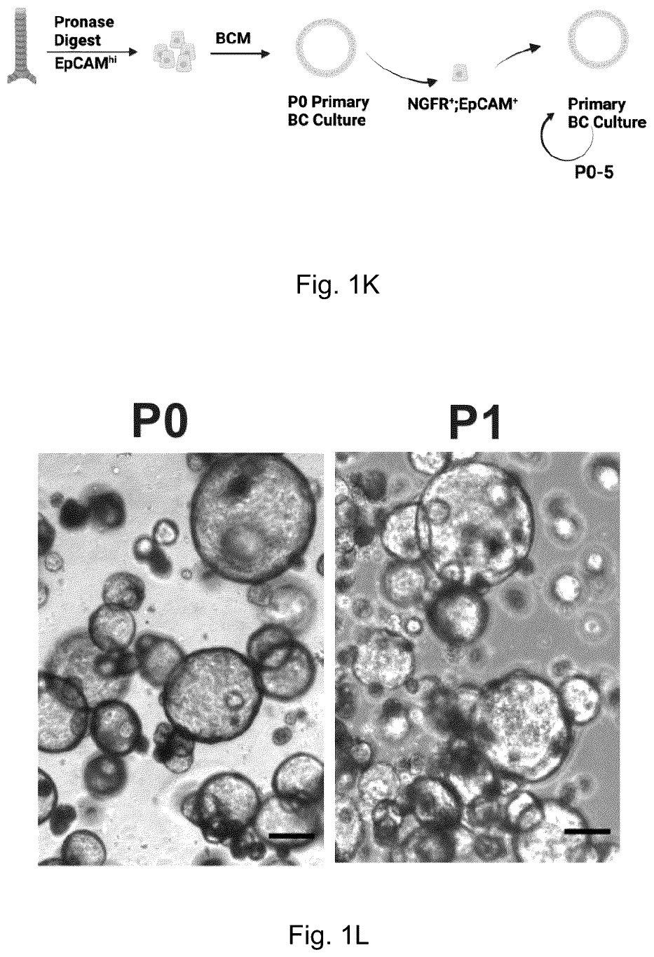

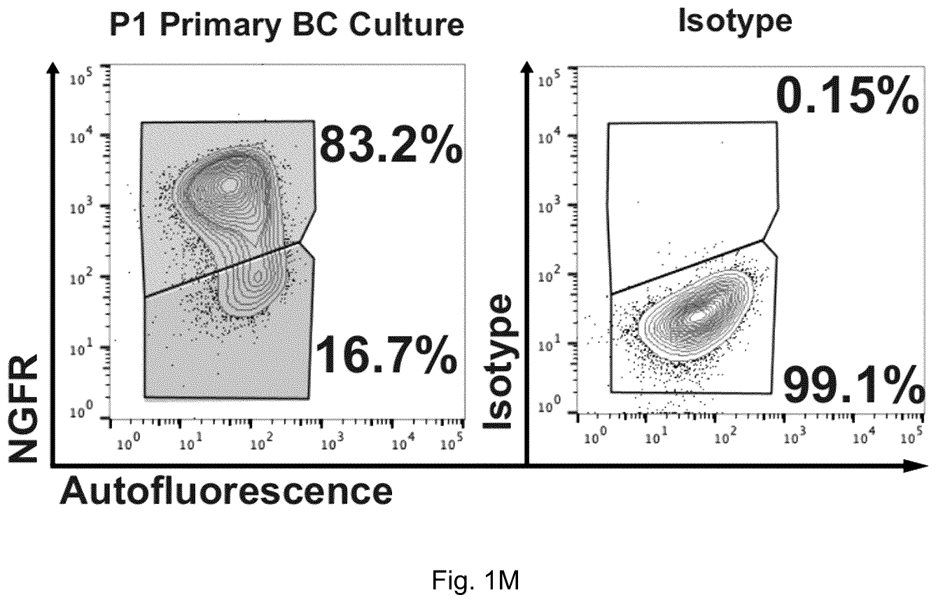

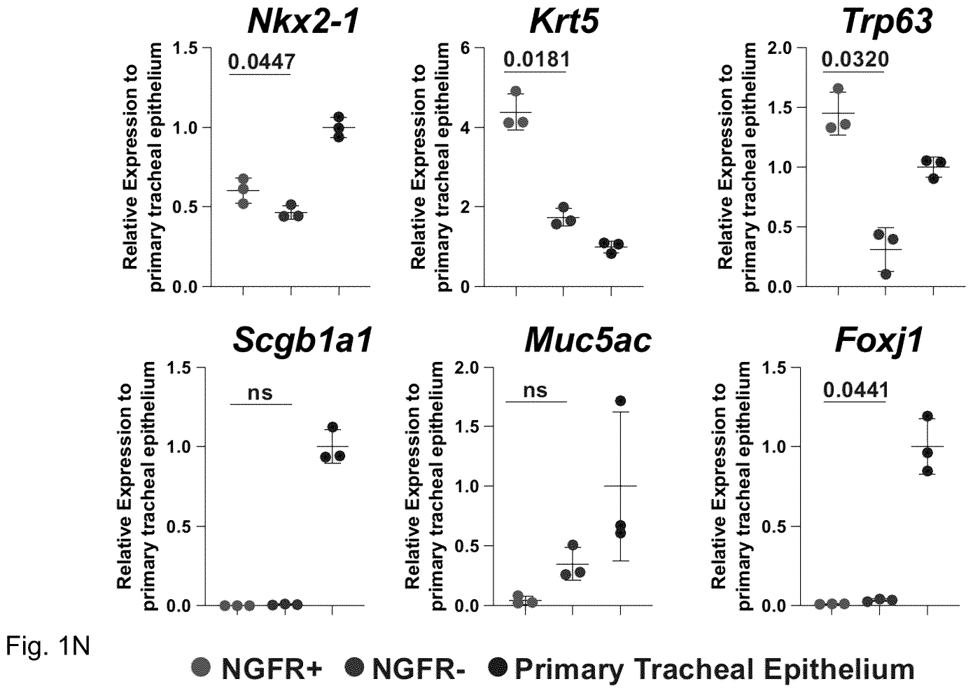

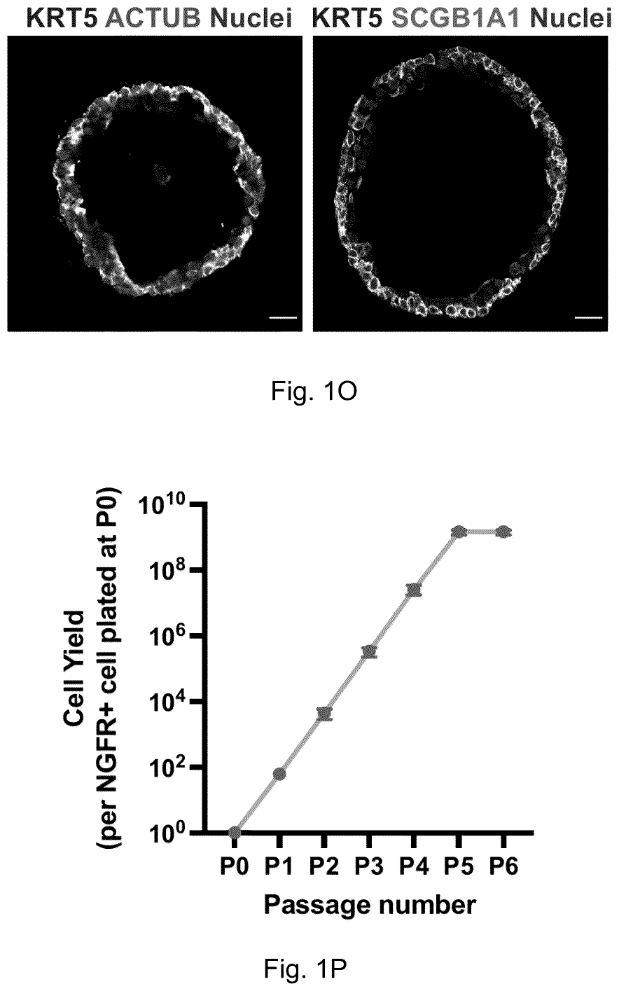

In some embodiments, the airway basal cells are primary cells. Primary cells are cells that are taken directly from living tissue, such as biopsied lung or tracheal material, and grown in vitro. Primary cells can be isolated using mechanical or enzymatic methods and placed in a controlled medium with nutrients and growth factors to support their proliferation. See, for example, Examples 2-3 or FIG. 1K for exemplary methods to prepare primary airway basal cells.

In some embodiments, the airway basal cells are differentiated from pluripotent stem cells. In some embodiments, the airway basal cells are differentiated from induced pluripotent stem cells. Such airway basal cells are differentiated pluripotent stem cells can be referred to herein as iBCs. See, for example, Examples 2-3 or FIG. 1A for exemplary methods to differentiate airway basal cells from pluripotent stem cells.

In some embodiments, the airway basal cells express at least one marker selected from the group consisting of: Tumor Protein P63 (TRP63), keratin-5 (KRT5), and NK2 Homeobox 1 (NKX2-1), or any combination thereof. In some embodiments, the airway basal cells express Tumor Protein P63 (TRP63). In some embodiments, the airway basal cells express keratin-5 (KRT5). In some embodiments, the airway basal cells express NK2 Homeobox 1 (NKX2-1). In some embodiments, the airway basal cells express TRP63 and KRT5. In some embodiments, the airway basal cells express TRP63 and NKX2-1. In some embodiments, the airway basal cells express KRT5 and NKX2-1. In some embodiments, the airway basal cells express TRP63, KRT5, and NKX2-1

In some embodiments, the airway basal cells are autologous to the subject, meaning derived from the subject themselves. In some embodiments, the airway basal cells are allogeneic to the subject, meaning the cells are derived from a donor other than the recipient subject. In some embodiments, the airway basal cells are mammalian. In some embodiments, the airway basal cells are human.

In some embodiments, the airway basal cells are engineered to reduce or prevent expression of at least one major histocompatibility complex (MHC) polypeptide. In some embodiments, the airway basal cells are engineered to reduce or prevent expression of at least one major histocompatibility complex (MHC) polypeptide on the cell surface of the airway basal cells. In some embodiments, the MHC polypeptide is an MHC class I polypeptide (e.g., HLA-A, HLA-B, and/or HLA-C) or an MHC class II polypeptide (e.g., HLA-DP, HLA-DM, HLA-DOA, HLA-DOB, HLA-DQ, and/or HLA-DR). In some embodiments, reduced MHC expression on the airway basal cells reduces immunogenicity of the airway basal cells. Such airway basal cells with reduced MHC expression can be referred to as “cloaked” cells.

In some embodiments, the airway basal cells are engineered to express at least one functional (e.g., wild-type, non-mutant) polypeptide that is dysfunctional (or has reduced functionality; e.g., mutant) in a respiratory tract disease. In some embodiments, the airway basal cells are engineered to express functional (e.g., wild-type, non-mutant) Cystic Fibrosis Transmembrane Conductance Regulator (CFTR) polypeptide, and the subject has cystic fibrosis. In some embodiments, the airway basal cells are engineered to express functional (e.g., wild-type, non-mutant) Dynein Axonemal Heavy Chain 5 (DNAH5) polypeptide and/or functional Dynein Axonemal Intermediate Chain 1 (DNAI1) polypeptide, and the subject has primary ciliary dyskinesia.

In some embodiments, the airway basal cells are administered at a dosage of at least 1×10⁶ cells per dose. In some embodiments, the airway basal cells are administered at a dosage of at least 1.5×10⁶ cells per dose. In some embodiments, the airway basal cells are administered at a dosage of 1×10⁴ to 1×10⁸, 1×10⁶ to 1.5×10⁶, 1×10⁶ to 2×10⁶, or 1.5×10⁶ to 2×10⁶ cells per dose. In some embodiments, the airway basal cells are administered at a dosage of about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶ cells per dose. In some embodiments, the airway basal cells are administered at a dosage of about 1×10⁴, about 1×10⁵, about 1×10⁶, about 1×10⁷, about 1×10⁸ cells per dose. In some embodiments, the airway basal cells are administered at a dosage of at least 1×10⁴, at least 1×10⁵, at least 1×10⁶, at least 1×10⁷, at least 1×10⁸ cells or more per dose.

### Compositions and Administration

The chemical agent for injuring respiratory tract epithelium described herein and/or the airway basal cells described herein can be comprised by compositions, such as pharmaceutical compositions, as described further herein. In one aspect, described herein is a pharmaceutical composition comprising a chemical agent as described herein and a pharmaceutically acceptable carrier. In one aspect, described herein is a pharmaceutical composition comprising airway basal cells as described herein and a pharmaceutically acceptable carrier.

### Formulations

In some embodiments, the technology described herein relates to a pharmaceutical composition comprising a chemical agent for removing respiratory tract epithelial cells as described herein, and optionally a pharmaceutically acceptable carrier. In some embodiments, the active ingredients of the pharmaceutical composition comprise a chemical agent as described herein. In some embodiments, the active ingredients of the pharmaceutical composition consist essentially of a chemical agent as described herein. In some embodiments, the active ingredients of the pharmaceutical composition consist of a chemical agent as described herein.

In some embodiments, the technology described herein relates to a pharmaceutical composition comprising airway basal cells as described herein, and optionally a pharmaceutically acceptable carrier. In some embodiments, the active ingredients of the pharmaceutical composition comprise airway basal cells as described herein. In some embodiments, the active ingredients of the pharmaceutical composition consist essentially of airway basal cells as described herein. In some embodiments, the active ingredients of the pharmaceutical composition consist of airway basal cells as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a chemical agent and/or airway basal cells as described herein.

In some embodiments, the pharmaceutical composition as described herein can be a parenteral dose form (i.e., administered or occurring elsewhere in the body than the mouth and alimentary canal). Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for inhalation or administration using a bronchoscope, dry products ready to be dissolved or suspended in a pharmaceutically acceptable vehicle for inhalation or administration using a bronchoscope, suspensions ready for inhalation or administration using a bronchoscope, and emulsions.

Suitable vehicles that can be used to provide parenteral dosage forms of pharmaceutical compositions as disclosed within are well known to those skilled in the art. Non-limiting examples include, without limitation: sterile water; water for injection USP; saline solution; glucose solution; aqueous vehicles such as but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Effective Amounts

In some embodiments, the methods described herein comprise administering an effective amount of compositions described herein, e.g., a chemical agent or bronchial thermoplasty to remove respiratory tract epithelial cells in a subject. As used herein, “removing epithelial cells from the respiratory tract” comprises lysing, killing, or otherwise damage epithelial cells of the respiratory tract such that they detach from the epithelium and/or expose the underlaying basement membrane. As compared with an equivalent untreated control, such removal of the respiratory tract epithelial cells is by at least 5%, 10%, 20%, 40%, 50%, 60%, 80%, 90%, 95%, 99% or more as measured by any standard technique. A variety of means for administering the compositions described herein to subjects are known to those of skill in the art.

The term “effective amount” as used herein can refer to the amount of the chemical agent or bronchial thermoplasty needed to remove a sufficient amount of respiratory tract epithelial cells to allow for engraftment of the airway basal cells. Thus, it is not generally practicable to specify an exact “effective amount” of the chemical agent. However, for any given case, an appropriate “effective amount” of the chemical agent can be determined by one of ordinary skill in the art using only routine experimentation.

In some embodiments, the methods described herein comprise administering an effective amount of compositions described herein, e.g. airway basal cells to a subject in order to alleviate a symptom of a respiratory tract disease or a respiratory tract injury. As used herein, “alleviating a symptom of a respiratory tract disease or a respiratory tract injury” is ameliorating any condition or symptom associated with the respiratory tract disease or the respiratory tract injury. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art.

The term “effective amount” as used herein refers to the amount of airway basal cells needed to alleviate at least one or more symptom of the respiratory tract disease or a respiratory tract injury, and relates to a sufficient amount of pharmacological composition to provide the desired effect. The term “therapeutically effective amount” therefore refers to an amount of airway basal cells that is sufficient to provide a particular anti-respiratory tract disease or anti-respiratory tract injury effect when administered to a typical subject. An effective amount as used herein, in various contexts, would also include an amount sufficient to delay the development of a symptom of the respiratory tract disease or respiratory tract injury, alter the course of a symptom of the respiratory tract disease or respiratory tract injury (for example but not limited to, slowing the progression of a symptom of the respiratory tract disease or respiratory tract injury), or reverse a symptom of the respiratory tract disease or respiratory tract injury. Thus, it is not generally practicable to specify an exact “effective amount” of airway basal cells. However, for any given case, an appropriate “effective amount” of airway basal cells can be determined by one of ordinary skill in the art using only routine experimentation.

Treatment according to the methods described herein can reduce levels of a marker or symptom of the respiratory tract disease or respiratory tract injury, by at least 10%, at least 15%, at least 20%, at least 25%, at least 30%, at least 40%, at least 50%, at least 60%, at least 70%, at least 80% or at least 90% or more.

In some embodiments, the effective amount of airway basal cells is at least 1×10⁶ cells per dose. In some embodiments, the effective amount of airway basal cells is at least 1.5×10⁶ cells per dose. In some embodiments, the effective amount of airway basal cells is 1×10⁴ to 1×10⁸, 1×10⁶ to 1.5×10⁶, 1×10⁶ to 2×10⁶, or 1.5×10⁶ to 2×10⁶ cells per dose. In some embodiments, the effective amount of airway basal cells is about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶ cells per dose. In some embodiments, the effective amount of airway basal cells is about 1×10⁴, about 1×10⁵, about 1×10⁶, about 1×10⁷, about 1×10⁸ cells per dose. In some embodiments, the effective amount of airway basal cells is at least 1×10⁴, at least 1×10⁵, at least 1×10⁶, at least 1×10⁷, at least 1×10⁸ cells or more per dose.

Effective amounts, toxicity, and therapeutic efficacy of the chemical agent and/or airway basal cells can be determined by standard pharmaceutical procedures in cell cultures or experimental animals, e.g., for determining the LD50 (the dose lethal to 50% of the population; e.g., for the chemical agent) and/or the ED50 (the dose therapeutically effective in 50% of the population; e.g., for the chemical agent and/or the airway basal cells). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a pulmonary concentration range that includes the ED50 as determined in cell culture. The effects of any particular dosage can be monitored by a suitable bioassay, e.g., for assessing respiratory tract epithelium damage and/or denudation. The dosage can be determined by a physician and adjusted, as necessary, to suit observed effects of the treatment.

In some embodiments, the effective amount of the chemical agent (e.g., a detergent or surfactant; e.g., polidocanol) is 0.5%-2%, 0.5%-1.75%, 0.5%-1.5%, 0.5%-1%, 0.5%-0.75%, 0.75%-2%, 0.75%-1.75%, 0.75%-1.5%, 0.75%-1%, 1%-2%, 1%-1.75%, 1%-1.5%, 1.5%-2%, 1.5%-1.75%, or 1.75%-2% in a solution. In some embodiments, the effective amount of the chemical agent (e.g., a detergent or surfactant; e.g., polidocanol) is at least 0.5%, at least 0.55%, at least 0.6%, at least 0.65%, at least 0.7%, at least 0.75%, at least 0.8%, at least 0.85%, at least 0.9%, at least 0.95%, at least 1%, at least 1.05%, at least 1.1%, at least 1.15%, at least 1.2%, at least 1.25%, at least 1.3%, at least 1.35%, at least 1.4%, at least 1.45%, at least 1.5%, at least 1.55%, at least 1.6%, at least 1.65%, at least 1.7%, at least 1.75%, at least 1.8%, at least 1.85%, at least 1.9%, at least 1.95%, or 2% in a solution.

In some embodiments, the effective amount of the chemical agent (e.g., a detergent or surfactant; e.g., polidocanol) is at most 0.5%, at most 0.55%, at most 0.6%, at most 0.65%, at most 0.7%, at most 0.75%, at most 0.8%, at most 0.85%, at most 0.9%, at most 0.95%, at most 1%, at most 1.05%, at most 1.1%, at most 1.15%, at most 1.2%, at most 1.25%, at most 1.3%, at most 1.35%, at most 1.4%, at most 1.45%, at most 1.5%, at most 1.55%, at most 1.6%, at most 1.65%, at most 1.7%, at most 1.75%, at most 1.8%, at most 1.85%, at most 1.9%, at most 1.95%, or at most 2% in a solution.

In some embodiments, the effective amount of the chemical agent (e.g., a detergent or surfactant; e.g., polidocanol) is about 0.5%, about 0.55%, about 0.6%, about 0.65%, about 0.7%, about 0.75%, about 0.8%, about 0.85%, about 0.9%, about 0.95%, about 1%, about 1.05%, about 1.1%, about 1.15%, about 1.2%, about 1.25%, about 1.3%, about 1.35%, about 1.4%, about 1.45%, about 1.5%, about 1.55%, about 1.6%, about 1.65%, about 1.7%, about 1.75%, about 1.8%, about 1.85%, about 1.9%, about 1.95%, or about 2% in a solution.

In some embodiments, an effective amount of the bronchial thermoplasty heats the epithelial of the respiratory tract to at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., at least 70° C., or more.

In some embodiments, an effective amount of the bronchial thermoplasty heats the epithelial of the respiratory tract to at most 45° C., at most 46° C., at most 47° C., at most 48° C., at most 49° C., at most 50° C., at most 51° C., at most 52° C., at most 53° C., at most 54° C., at most 55° C., at most 56° C., at most 57° C., at most 58° C., at most 59° C., at most 60° C., at most 61° C., at most 62° C., at most 63° C., at most 64° C., at most 65° C., at most 66° C., at most 67° C., at most 68° C., at most 69° C., or at most 70° C.

In some embodiments, an effective amount of the bronchial thermoplasty heats the epithelial of the respiratory tract to about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., 45° C. to 50° C., 50° C. to 55° C., 55° C. to 60° C., 60° C. to 65° C., or 65° C. to 70° C.

In some embodiments, an effective amount of the bronchial thermoplasty heats the epithelial of the respiratory tract for a total of at least 10 seconds, at least 20 seconds, at least 30 seconds, at least 40 seconds, at least 50 seconds, at least 60 seconds, at least 70 seconds, at least 80 seconds, at least 90 seconds, at least 100 seconds, at least 110 seconds, at least 120 seconds, at least 3 minutes, at least 4 minutes, at least 5 minutes, at least 6 minutes, at least 7 minutes, at least 8 minutes, at least 9 minutes, at least 10 minutes, or more.

The dosage ranges for the administration of the chemical agent or bronchial thermoplasty, according to the methods described herein depend upon, for example, the form of the chemical agent or bronchial thermoplasty, its potency, and the extent to which the respiratory tract epithelial cells are desired to be removed, for example the percentage denudation of the respiratory tract epithelium. The dosage of the chemical agent or bronchial thermoplasty should not be so large as to cause adverse side effects, such as difficulty breathing, infection, or death. Generally, the dosage of the chemical agent or bronchial thermoplasty can vary with the age, condition, and sex of the patient and can be determined by one of skill in the art. The dosage can also be adjusted by the individual physician in the event of any complication.

The dosage ranges for the administration of the airway basal cells, according to the methods described herein depend upon, for example, the form of the airway basal cells, their potency, and the extent to which symptoms, markers, or indicators of a condition described herein are desired to be reduced, for example the percentage reduction desired for the respiratory tract disease or the respiratory tract injury. The dosage of the airway basal cells should not be so large as to cause adverse side effects, such as an adverse immune reaction. Generally, the dosage of the airway basal cells can vary with the age, condition, and sex of the patient and can be determined by one of skill in the art. The dosage can also be adjusted by the individual physician in the event of any complication.

The efficacy of the chemical agents, bronchial thermoplasty, and airway basal cells, in, e.g. the treatment of a respiratory tract disease or respiratory tract injury, or to induce a response as described herein (e.g. respiratory tract epithelium injury) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a respiratory tract disease or respiratory tract injury treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms; or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters.

In vitro and animal model assays allow the assessment of a given dose of a chemical agent or bronchial thermoplasty for removing respiratory tract epithelial cells, as described herein, and/or a given dose of airway basal cells described herein.

### Administration

In some embodiments, the step of administering the airway basal cells occurs after the step of removing epithelial cells from the respiratory tract. In some embodiments, the step of administering the airway basal cells occurs at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours after the step of removing epithelial cells from the respiratory tract.

In some embodiments, the step of administering the airway basal cells occurs at most 3 hours, at most 4 hours, at most 5 hours, at most 6 hours, at most 7 hours, at most 8 hours, at most 9 hours, at most 10 hours, at most 11 hours, at most 12 hours, at most 13 hours, at most 14 hours, at most 15 hours, at most 16 hours, at most 17 hours, at most 18 hours, at most 19 hours, at most 20 hours, at most 21 hours, at most 22 hours, at most 23 hours, or at most 24 hours after the step of removing epithelial cells from the respiratory tract.

In some embodiments, the step of administering the airway basal cells occurs about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after the step of removing epithelial cells from the respiratory tract. In some embodiments, the step of administering the airway basal cells occurs 3 hours to 6 hours, 6 hours to 12 hours, 12 hours to 18 hours, or 18 hours to 24 hours after the step of removing epithelial cells from the respiratory tract.

In some embodiments, the step of administering the airway basal cells occurs concurrently with the step of removing epithelial cells from the respiratory tract.

In some embodiments, the chemical agent to injure the epithelium is administered to the trachea, at least one bronchus, and/or at least one bronchiole of the respiratory tract. In some embodiments, the chemical agent to injure the epithelium is administered to the trachea. In some embodiments, the chemical agent to injure the epithelium is administered to at least one bronchus. In some embodiments, the chemical agent to injure the epithelium is administered to at least one bronchiole. In some embodiments, the chemical agent to injure the epithelium is administered to the trachea and at least one bronchus. In some embodiments, the chemical agent to injure the epithelium is administered to the trachea and at least one bronchiole. In some embodiments, the chemical agent to injure the epithelium is administered to at least one bronchus and at least one bronchiole. In some embodiments, the chemical agent to injure the epithelium is administered to the trachea, at least one bronchus, and at least one bronchiole. In some embodiments, the chemical agent to injure the epithelium is administered to at least one alveolus.

In some embodiments, the airway basal cells are administered to the trachea, at least one bronchus, and/or at least one bronchiole of the respiratory tract. In some embodiments, the airway basal cells are administered to the trachea. In some embodiments, the airway basal cells are administered to at least one bronchus. In some embodiments, the airway basal cells are administered to at least one bronchiole. In some embodiments, the airway basal cells are administered to the trachea and at least one bronchus. In some embodiments, the airway basal cells are administered to the trachea and at least one bronchiole. In some embodiments, the airway basal cells are administered to at least one bronchus and at least one bronchiole. In some embodiments, the airway basal cells are administered to the trachea, at least one bronchus, and at least one bronchiole. In some embodiments, the airway basal cells are administered to at least one alveolus.

In some embodiments, the chemical agent and/or the airway basal cells are administered using inhalation. In some embodiments, the chemical agent is administered using inhalation. In some embodiments, the airway basal cells are administered using inhalation. In some embodiments, the chemical agent and the airway basal cells are administered using inhalation.

In some embodiments, the chemical agent and/or the airway basal cells are administered using an inhalation device. In some embodiments, the chemical agent is administered using an inhalation device. In some embodiments, the airway basal cells are administered using an inhalation device. In some embodiments, the chemical agent and the airway basal cells are administered using an inhalation device. Non-limiting examples of inhalation devices include: a bronchoscope, a micro-bronchoscope, a nebulizer, or an inhaler.

In some embodiments, the chemical agent and/or the airway basal cells are administered using a bronchoscope. In some embodiments, the chemical agent is administered using a bronchoscope. In some embodiments, the airway basal cells are administered using a bronchoscope. In some embodiments, the chemical agent and the airway basal cells are administered using a bronchoscope.

In some embodiments, the chemical agent and/or the airway basal cells are administered using a micro-bronchoscope. In some embodiments, the chemical agent is administered using a micro-bronchoscope. In some embodiments, the airway basal cells are administered using a micro-bronchoscope. In some embodiments, the chemical agent and the airway basal cells are administered using a micro-bronchoscope.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen.

In certain embodiments, an effective dose of a composition comprising the chemical agent and/or the airway basal cells as described herein can be administered to a patient once. In certain embodiments, an effective dose of a composition comprising the chemical agent and/or the airway basal cells can be administered to a patient repeatedly.

The dosing schedule can vary from once a week to daily depending on a number of clinical factors, such as the subject's sensitivity to the chemical agent and/or the airway basal cells. The desired dose or amount can be administered at one time or divided into subdoses, e.g., 2-4 subdoses and administered over a period of time, e.g., at appropriate intervals through the day or other appropriate schedule. In some embodiments, administration can be one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising the chemical agent and/or the airway basal cells can be administered over a period of time, such as over a 5 minute, 10 minute, 15 minute, 20 minute, or 25 minute period.

In some embodiments, after an initial treatment regimen, the treatments can be administered on a less frequent basis. For example, after treatment biweekly for three months, treatment can be repeated once per month, for six months or a year or longer.

In some embodiments of any of the aspects, the chemical agent and the airway basal cells described herein is administered as a monotherapy, e.g., another treatment for the respiratory tract disease or respiratory tract injury is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include a cancer therapy selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gammalI and calicheamicin omegaIl (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation (e.g., pazopanib, sunitinib, sorafenib, regorafenib, cabozantinib, lenvatinib, ponatinib, ziv-aflibercept, axitinib, tivozanib, vandetanib, ramucirumab); and pharmaceutically acceptable salts, acids or derivatives of any of the above.

In some embodiments of any of the aspects, the cancer treatment method further comprises administering an immune checkpoint inhibitor. In some embodiments of any of the aspects, the immune checkpoint inhibitor comprises an immune checkpoint inhibitor antibody. In some embodiments of any of the aspects, the checkpoint inhibitor immunotherapy is an inhibitor of a checkpoint molecule selected from the group consisting of: programmed cell death 1 (PD-1), programmed death-ligand 1 (PD-L1), cytotoxic T-lymphocyte-associated protein 4 (CTLA-4), Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG-3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).

Non-limiting examples of immune checkpoint inhibitors (ICIs) include: pembrolizumab (Keytruda®), nivolumab (Opdivo®), cemiplimab (Libtayo®), spartalizumab, camrelizumab (AiRuiKa™), sintilimab (TYVYT®), tislelizumab, toripalimab (Tuoyi™) dostarlimab (JEMPERLI), INCMGA00012, AMP-224, AMP-514 (MEDI0608), atezolizumab (Tecentriq®), avelumab (Bavencio®), envafolimab (KN035), cosibelimab (CK-301), AUNP12, CA-170, BMS-986189, BMS-936559 (MDX-1105), durvalumab (IMIFINZI®), tremelimumab, and ipilimumab (Yervoy®). See e.g., U.S. Pat. Nos. 5,811,097, 5,855,887, 6,051,227, 6,682,736, 6,984,720, 7,595,048, 7,605,238, 7,943,743, 8,008,449, 8,217,149, 8,354,509, 8,383,796, 8,728,474, 8,735,553, 8,779,105, 8,779,108, 8,907,053, 8,900,587, 8,952,136, 9,067,999, 9,073,994, 9,683,048, 9,987,500, 10,160,736, 10,316,089, 10,441,655, 10,590,199, 11,225,522, US Patent Publication US2014341917; Storz et al., MAbs. 2016 January; 8(1): 10-26; the contents of each of which are incorporated herein by reference in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

### Engraftment Methods

In multiple aspects, described herein are methods of engrafting airway basal cells into the respiratory tract of a subject in need thereof. In one aspect, the method comprises: (a) removing epithelial cells from the respiratory tract; and (b) administering airway basal cells to the respiratory tract. In some embodiments, the airway basal cells are administered at or near the site where epithelial cells were removed from the respiratory tract.

In some embodiments, the subject is healthy. In some embodiments, the subject has a respiratory tract disease or a respiratory tract injury, non-limiting examples of which are provided herein.

In some embodiments, the engrafted airway basal cells reconstitute the tissue-resident airway basal stem cell compartment in the respiratory tract of the subject.

In some embodiments, the engrafted airway basal cells replace at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more of the epithelium in the respiratory tract of the subject.

In some embodiments, the engrafted airway basal cells or their progeny comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or more of the epithelium in the respiratory tract of the subject, after the engraftment.

In some embodiments, the engrafted airway basal cells (or their progeny) persist for at least 1 month, at least 6 months, at least 1 year, at least 1.5 years, at least 2 years, at least 3 years, at least 4 years, at least 5 years, or more following the engraftment.

In some embodiments, the engrafted airway basal cells exhibit self-renewal and/or multipotent differentiation capacity for at least 1 month, at least 6 months, at least 1 year, at least 1.5 years, at least 2 years, at least 3 years, at least 4 years, at least 5 years, or more years following the engraftment.

In some embodiments, the multipotent differentiation capacity comprises ability to differentiate into any one of the following lineages: basal cell, secretory cell, ciliated cell, ionocyte, neuroendocrine cell, tuft cell, and/or hillock cell airway epithelial lineages.

In some embodiments, the engrafted airway basal cells exhibit a transcriptome that is at least 70%, at least 75%, at least 80%, at least 85%, at least 90%, at least 95%, at least 99% or more identical to the transcriptome of endogenous airway epithelium cells.

### Treatment Methods

In multiple aspects, described herein are methods of treating a respiratory tract disease or a respiratory tract injury. In one aspect, described herein is a method of treating a respiratory tract disease, the method comprising: (a) removing epithelial cells from the respiratory tract of a subject in need thereof; and (b) administering an effective amount of airway basal cells to the respiratory tract. In some embodiments, the respiratory tract disease is selected from the group consisting of: cystic fibrosis; primary ciliary dyskinesia; idiopathic pulmonary fibrosis (IPF); chronic obstructive pulmonary disease (COPD); lung cancer (e.g., lung nodules, non-small cell lung cancer, small cell lung cancer, or mesothelioma); asthma; chronic bronchitis; bronchiectasis; bronchiolitis obliterans syndrome; and chronic lung allograft disorder (CLAD).

In one aspect, described herein is a method of treating a respiratory tract injury, the method comprising: (a) removing epithelial cells from the respiratory tract of a subject in need thereof; and (b) administering an effective amount of airway basal cells to the respiratory tract. In some embodiments, the respiratory tract injury is associated with: inhalation of a chemical (e.g., an acid), smoke, fire, and/or a heated substance; a burn; radiation damage; chemotherapy damage; space travel; a respiratory infection (e.g., viral, bacterial, fungal); a birth defect; a lung cancer; and/or a surgery (e.g., tracheal resection, lung lobectomy).

In some embodiments, the methods described herein relate to treating a subject having or diagnosed as having a respiratory tract disease or a respiratory tract injury. Subjects having a respiratory tract disease or a respiratory tract injury can be identified by a physician using current methods of diagnosing a respiratory tract disease or a respiratory tract injury. Symptoms and/or complications of a respiratory tract disease or a respiratory tract injury which characterize these conditions and aid in diagnosis are well known in the art. A family history of a respiratory tract disease or a respiratory tract injury, or exposure to risk factors for a respiratory tract disease or a respiratory tract injury can also aid in determining if a subject is likely to have a respiratory tract disease or a respiratory tract injury or in making a diagnosis of a respiratory tract disease or a respiratory tract injury.

### Kits

Another aspect of the technology described herein relates to kits, e.g., for treating a respiratory tract disease or a respiratory tract injury, among others. Described herein are kit components that can be included in one or more of the kits described herein. In one aspect, described herein is a kit comprising: (a) a chemical agent to remove epithelial cells from a respiratory tract; and/or (b) airway basal cells. In some embodiments, the components described herein can be provided singularly or in any combination as a kit.

In some embodiments, the kit comprises an effective amount of the chemical agent and/or airway basal cells. As will be appreciated by one of skill in the art, the chemical agent can be supplied in a lyophilized form or a concentrated form that can diluted or suspended in liquid prior to use.

In addition, the kit optionally comprises informational material. The informational material can be descriptive, instructional, marketing or other material that relates to the methods described herein. The informational material of the kits is not limited in its form. In one embodiment, the informational material can include information about concentration, date of expiration, batch or production site information, and so forth. In one embodiment, the informational material relates to methods for using or administering the components of the kit.

In some embodiments, the compositions in the kit can be provided in a watertight or gas tight container which in some embodiments is substantially free of other components of the kit. For example, the chemical agent and/or airway basal cells can be supplied in more than one container, e.g., they can be supplied in a container having sufficient amount for a predetermined number of administrations, e.g., 1, 2, 3 or greater. One or more components as described herein can be provided in any form, e.g., liquid, dried or lyophilized form. It is preferred that the components described herein are substantially pure and/or sterile. When the airway basal cells described herein are provided in a liquid solution, the liquid solution preferably is an aqueous solution, with a sterile aqueous solution being preferred.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a respiratory tract disease or a respiratory tract injury. A subject can be male or female.

A subject can be one who has been previously diagnosed with or identified as suffering from or having a condition in need of treatment (e.g. a respiratory tract disease or a respiratory tract injury) or one or more complications related to such a condition, and optionally, have already undergone treatment for a respiratory tract disease or a respiratory tract injury or the one or more complications related to a respiratory tract disease or a respiratory tract injury. Alternatively, a subject can also be one who has not been previously diagnosed as having a respiratory tract disease or a respiratory tract injury or one or more complications related to a respiratory tract disease or a respiratory tract injury. For example, a subject can be one who exhibits one or more risk factors for a respiratory tract disease or a respiratory tract injury or one or more complications related to a respiratory tract disease or a respiratory tract injury or a subject who does not exhibit risk factors.

A “subject in need” of treatment for a particular condition can be a subject having that condition, diagnosed as having that condition, or at risk of developing that condition.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a respiratory tract disease or a respiratory tract injury. The term “treating” includes reducing or alleviating at least one adverse effect or symptom of a condition, disease or disorder associated with a respiratory tract disease or a respiratory tract injury. Treatment is generally “effective” if one or more symptoms or clinical markers are reduced. Alternatively, treatment is “effective” if the progression of a disease or injury is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease or injury, delay or slowing of disease or injury progression, amelioration or palliation of the disease or injury state, remission (whether partial or total), and/or decreased mortality, whether detectable or undetectable. The term “treatment” of a disease or injury also includes providing relief from the symptoms or side-effects of the disease or injury (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a composition or compound as disclosed herein into a subject by a method or route which results in at least partial delivery of the composition, compound, or metabolite thereof at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine. “Contacting” of a cell can be performed in vitro, ex vivo, or in vivo.

In some embodiments of any of the aspects, the cells can be maintained in culture. As used herein, “maintaining” refers to continuing the viability of a cell or population of cells. A maintained population of cells will have at least a subpopulation of metabolically active cells.

The term “statistically significant” or “significantly” refers to statistical significance and generally means a two standard deviation (2SD) or greater difference or a p-value of less than 0.05.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A method of engrafting airway basal cells into the respiratory tract of a subject in need thereof, the method comprising:
   (a) removing epithelial cells from the respiratory tract; and
   (b) administering airway basal cells to the respiratory tract.
2. The method of paragraph 1, wherein the epithelium cells are removed from the respiratory tract a chemical agent or using bronchial thermoplasty.
3. The method of paragraph 2, wherein the chemical agent comprises a detergent.
4. The method of paragraph 2 or 3, wherein the chemical agent is selected from the group consisting of: polidocanol; 3-[(3-cholamidopropyl)dimethylammonio]-1-propane sulfonate (CHAPS); lucinactant, colfosceril palmitate, beractant, calefacient, and poractant alfa.
5. The method of any one of paragraphs 2-3, wherein the chemical agent comprises polidocanol.
6. The method of any one of paragraphs 1-5, wherein step (b) of administering the airway basal cells occurs 3 hours to 24 hours after step (a) of removing epithelium epithelial cells from of the respiratory tract.
7. The method of any one of paragraphs 1-6, wherein the epithelial cells are removed in the trachea, at least one bronchus, and/or at least one bronchiole of the respiratory tract.
8. The method of any one of paragraphs 1-7, wherein the airway basal cells are administered to the trachea, at least one bronchus, and/or at least one bronchiole of the respiratory tract.
9. The method of any one of paragraphs 2-8, wherein the chemical agent and/or the airway basal cells are administered using a bronchoscope.
10. The method of any one of paragraphs 1-9, wherein the airway basal cells are primary cells.
11. The method of any one of paragraphs 1-10, wherein the airway basal cells are differentiated from pluripotent stem cells.
12. The method of any one of paragraphs 1-11, wherein the airway basal cells express at least one marker selected from the group consisting of: Tumor Protein P63 (TRP63), keratin-5 (KRT5), and NK2 Homeobox 1 (NKX2-1).
13. The method of any one of paragraphs 1-12, wherein the airway basal cells are autologous or allogeneic to the subject.
14. The method of any one of paragraphs 1-13, wherein the airway basal cells are engineered to reduce or prevent expression of at least one major histocompatibility complex (MHC) polypeptide to reduce immunogenicity of the airway basal cells.
15. The method of any one of paragraphs 1-14, wherein the subject is healthy.
16. The method of any one of paragraphs 1-14, wherein the subject has a respiratory tract disease or a respiratory tract injury.
17. The method of paragraph 16, wherein the respiratory tract disease is selected from the group consisting of: cystic fibrosis; primary ciliary dyskinesia; idiopathic pulmonary fibrosis (IPF); chronic obstructive pulmonary disease (COPD); lung cancer; asthma; chronic bronchitis; bronchiectasis; bronchiolitis obliterans syndrome; and chronic lung allograft disorder (CLAD).
18. The method of paragraph 16, wherein the respiratory tract injury is associated with: inhalation of a chemical (e.g., an acid), smoke, fire, and/or a heated substance; a burn; radiation damage; chemotherapy damage; space travel; a respiratory infection; a birth defect; a lung cancer; and/or a surgery (e.g., tracheal resection, lung lobectomy).
19. The method of any one of paragraphs 1-18, wherein the airway basal cells are engineered to express at least one functional polypeptide that is dysfunctional in a respiratory tract disease.
20. The method of paragraph 19, wherein the airway basal cells are engineered to express functional Cystic Fibrosis Transmembrane Conductance Regulator (CFTR) polypeptide, and the subject has cystic fibrosis.
21. The method of paragraph 19, wherein the airway basal cells are engineered to express functional Dynein Axonemal Heavy Chain 5 (DNAH5) polypeptide and/or functional Dynein Axonemal Intermediate Chain 1 (DNAI1) polypeptide, and the subject has primary ciliary dyskinesia.
22. The method of any one of paragraphs 1-21, wherein the engrafted airway basal cells reconstitute the tissue-resident airway basal stem cell compartment in the respiratory tract of the subject.
23. The method of any one of paragraphs 1-22, wherein the engrafted airway basal cells comprise at least 10% of the epithelium in the respiratory tract of the subject after the engraftment.
24. The method of any one of paragraphs 1-23, wherein the engrafted airway basal cells persist for at least 2 years following the engraftment.
25. The method of any one of paragraphs 1-24, wherein the engrafted airway basal cells exhibit self-renewal and/or multipotent differentiation capacity for at least 2 years following the engraftment.
26. The method of paragraph 25, wherein the multipotent differentiation capacity comprises: basal cell, secretory cell, ciliated cell, ionocyte, neuroendocrine cell, tuft cell, and/or hillock cell airway epithelial lineages.
27. The method of any one of paragraphs 1-26, wherein the engrafted airway basal cells exhibit a transcriptome that is at least 80% identical to the transcriptome of endogenous airway epithelium cells.
28. A method of treating a respiratory tract disease, the method comprising:
   (a) removing epithelial cells from the respiratory tract of a subject in need thereof, and
   (b) administering an effective amount of airway basal cells to the respiratory tract;
   wherein the respiratory tract disease is selected from the group consisting of: cystic fibrosis; primary ciliary dyskinesia; idiopathic pulmonary fibrosis (IPF); chronic obstructive pulmonary disease (COPD); lung cancer; asthma; chronic bronchitis; bronchiectasis; bronchiolitis obliterans syndrome; and chronic lung allograft disorder (CLAD).
29. A method of treating a respiratory tract injury, the method comprising:
   (a) removing epithelial cells from the respiratory tract of a subject in need thereof, and
   (b) administering an effective amount of airway basal cells to the respiratory tract;
   wherein the respiratory tract injury is associated with: inhalation of a chemical (e.g., an acid), smoke, fire, and/or a heated substance; a burn; radiation damage; chemotherapy damage; space travel; a respiratory infection; a birth defect; a lung cancer; and/or a surgery (e.g., tracheal resection, lung lobectomy).
30. A kit comprising:
   (a) a chemical agent to remove epithelial cells from a respiratory tract; and
   (b) airway basal cells.

The technology described herein is further illustrated by the following examples which in no way should be construed as being further limiting.

### EXAMPLES

### Example 1: Airway Engraftment of Pluripotent Stem Cell-Derived Basal Stem Cells

Lung diseases resulting from airway epithelial dysfunction are responsible for significant morbidity and mortality. In vivo engraftment of exogenously derived airway stem cells generated from pluripotent stem cells (PSC) provides an autologous cell-based therapy for airway diseases such as cystic fibrosis and primary ciliary dyskinesia. Described herein is a model of airway transplantation for use in airway cell-based therapies. The approach described herein: 1) generated mouse pluripotent stem cell-derived airway basal stem cells (iBCs); 2) developed the method to efficiently engraft mouse iBCs into the stem cell compartment of the syngeneic immunocompetent mouse trachea, which resulted in life-long, functional grafted epithelium after one dose of cell delivery and functional reconstitution of the airway stem cell compartment in vivo (e.g. life long self renewing stem cells that also undergo trilineage in vivo differentiation to the major cell types of the airway: basal, secretory, and ciliated cells; 3) utilized the mouse airway transplantation approach to engraft human iBCs (see e.g., U.S. Pat. Nos. 10,590,392 B2; 11,366,115 B2; 10,975,357 B2; the contents of each of which are incorporated herein by reference in their entireties) in immunodeficient mouse trachea, which resulted in functional grafted epithelium after one dose of cell delivery; 4) in an adaptation to primary cells, the described method was also applied to similarly graft primary mouse or primary human basal cells. These primary cells were obtained from tracheas, expanded ex vivo in cell culture, and then successfully transplanted into injured airways in vivo.

The described methods and compositions are useful as therapies for airway epithelial diseases. 1) The mouse-to-mouse transplantation method demonstrates autologous stem cell therapy in the airway, which can alleviate the need for immunosuppression if autologous (syngeneic) cells are delivered (either with or without ex vivo gene transfer and/or gene-editing modifications prior to transplant). 2) The mouse-to-mouse transplantation method also allows studies of allogeneic transplantation, which allows researchers to study the immune response to grafted airway epithelial cells, which is not possible with other immunodeficient transplantation models. This also facilitates the development of a hypoimmunogenic product (e.g. “cloaked cells” that can be used as an off-the shelf therapeutic product. 3) Polidocanol, an FDA-approved agent, was used for airway epithelial injury prior to cell delivery in the transplantation model, which can be used in clinics via bronchoscopic delivery for local removal of diseased epithelium prior to transplantation of healthy donor cells. Previous airway/lung injury models have used naphthalene or bleomycin, both of which have systemic toxicity and are suitable for therapeutic use. 4) The human-to-mouse transplantation model established an in vivo platform to test human donor cell types that can be used as a therapeutic cell-based product.

The methods and compositions described herein demonstrate the following results: 1a) engraftment of mouse and human iBCs into the stem cell compartment of the airway epithelium in vivo; 1b) engraftment of primary mouse or human basal cells. into the stem cell compartment of the airway epithelium in vivo; 2) utilization of an immunocompetent host in airway transplantation, which allows modeling of autologous or allogeneic transplantation studies; 3) utilization of polidocanol in the airway to create localized injury and niche prior to the transplantation of iBCs.

Any variation in iBC type is contemplated herein: e.g. beyond autologous therapies, a “cloaked” (hypoimmune) cell can be engineered by gene editing to serve as an off the shelf allogeneic product. A variety of other gene editing approaches can be used to modify the iPSCs or iBCs prior to transplant for the correction of genetic airway diseases or the modulation of endogenous signaling pathways if cells were engineered to secrete certain factors. Without wishing to be bound by theory, it is contemplated herein that grafting these cells beyond the large airways (e.g., into the small airways) can treat distal lung diseases such as idiopathic pulmonary fibrosis (IPF) or chronic obstructive pulmonary disease (COPD). One modification/adaptation of the iBC method is application of these identical methods, but with primary basal cells harvested from the human airways and expanded/cultured ex vivo (along with/without gene editing/gene transfer) prior to transplantation into the injured airways to reconstitute the airway stem cell compartment in vivo.

### Example 2: Airway Stem Cell Reconstitution by Transplantation of Primary or Pluripotent Stem Cell-Derived Basal Cells

Life-long reconstitution of a tissue's resident stem cell compartment with engrafted cells can durably replenish organ function. Demonstrated herein is the engraftment of the airway epithelial stem cell compartment via intra-airway transplantation of mouse or human primary and pluripotent stem cell (PSC)-derived airway basal cells (BCs). Murine primary or PSC-derived BCs transplanted into polidocanol-injured syngeneic recipients gave rise for at least two years to progeny that stably displayed the morphologic, molecular, and functional phenotypes of airway epithelia. The engrafted basal-like cells retain extensive self-renewal potential, evident by the capacity to reconstitute the tracheal epithelium through seven generations of secondary transplantation. Using the same approach, human primary or PSC-derived BCs transplanted into NSG recipient mice similarly display multilineage airway epithelial differentiation in vivo. These methods can be used as a syngeneic cell-based therapy for patients with diseases resulting from airway epithelial cell damage or dysfunction.

### Results:

### Generation and Maintenance of Murine Primary and PSC-Derived Basal Cells In Vitro

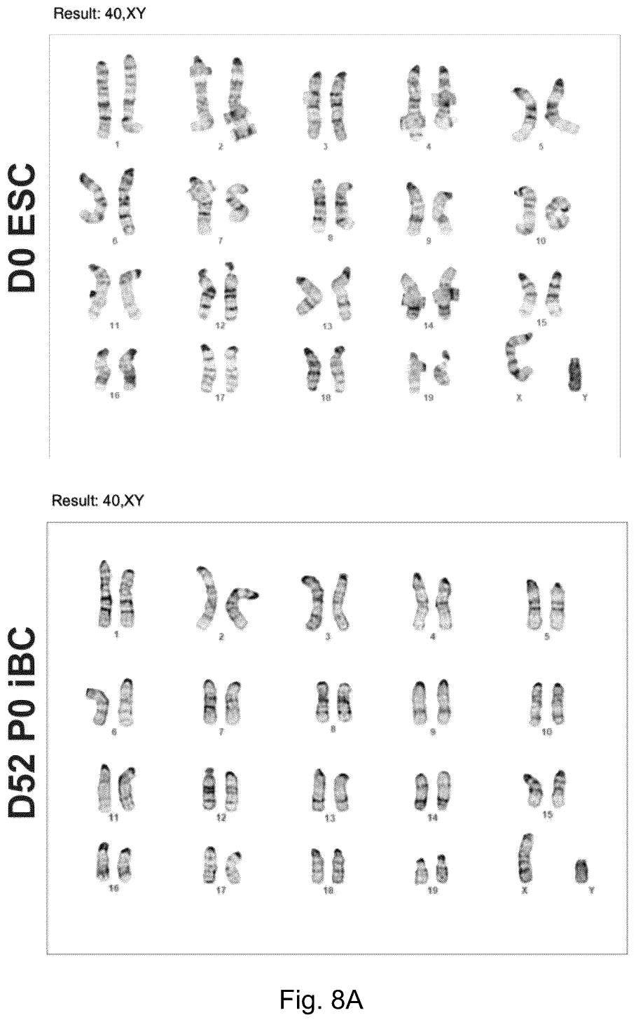

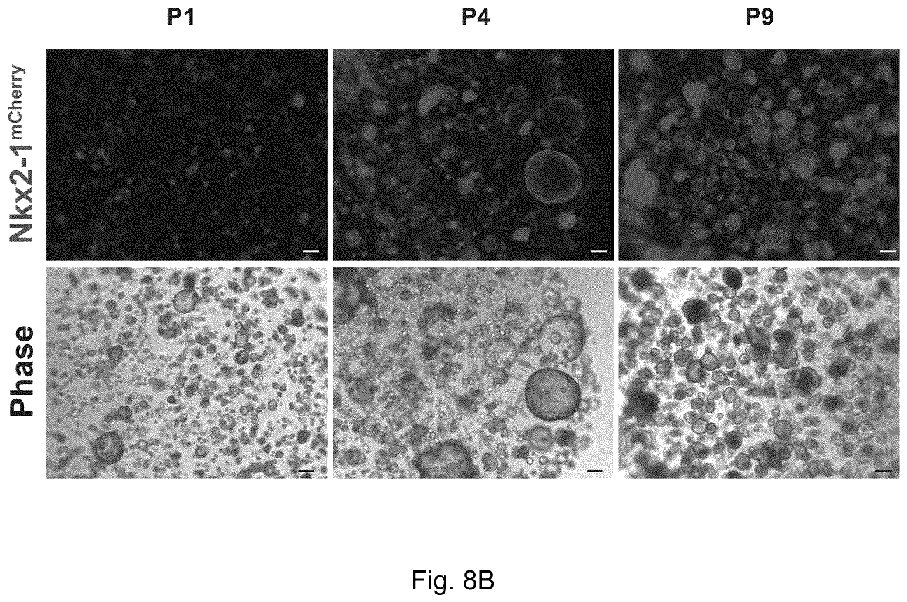

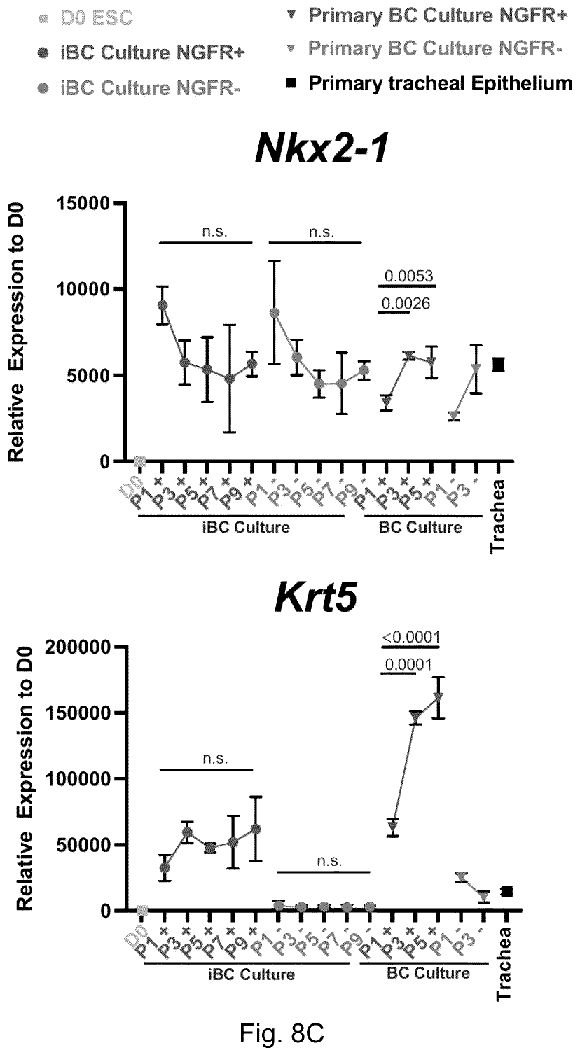

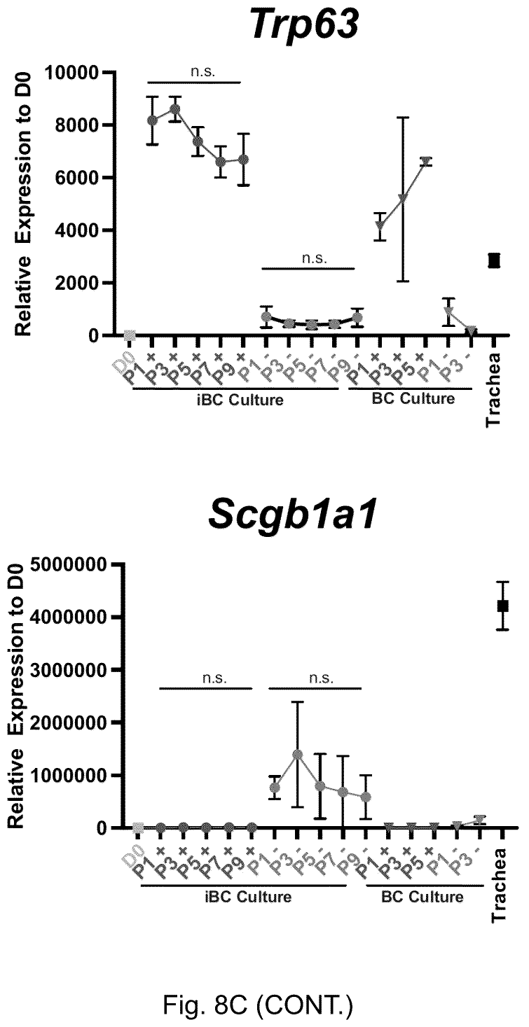

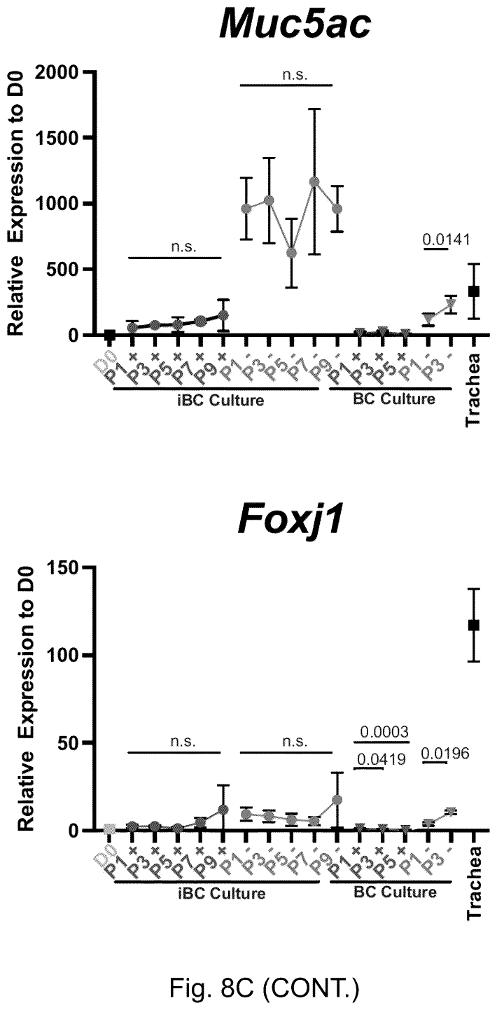

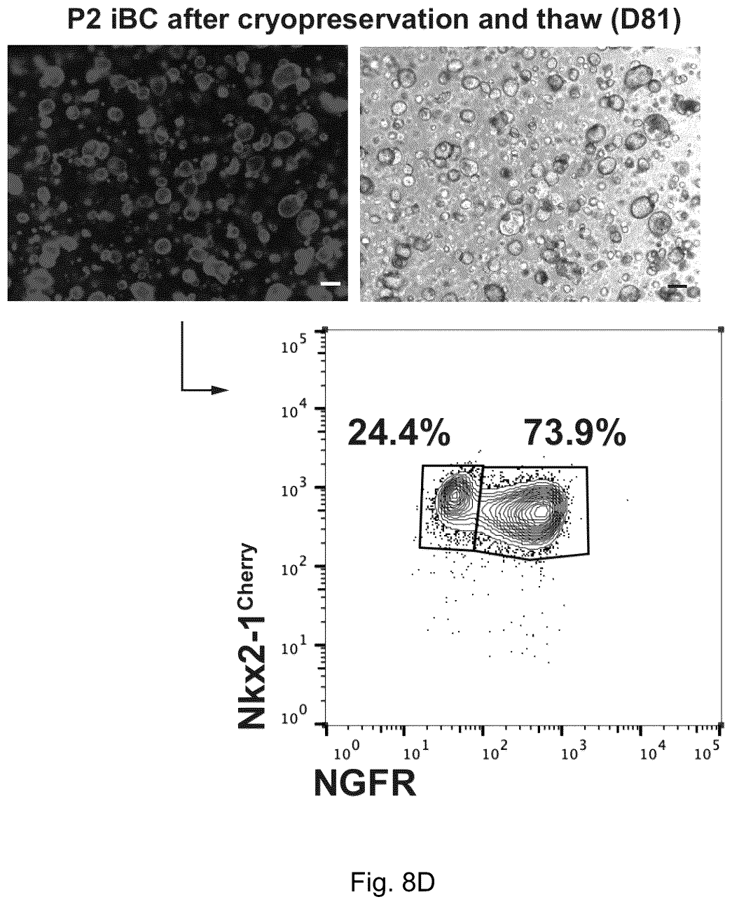

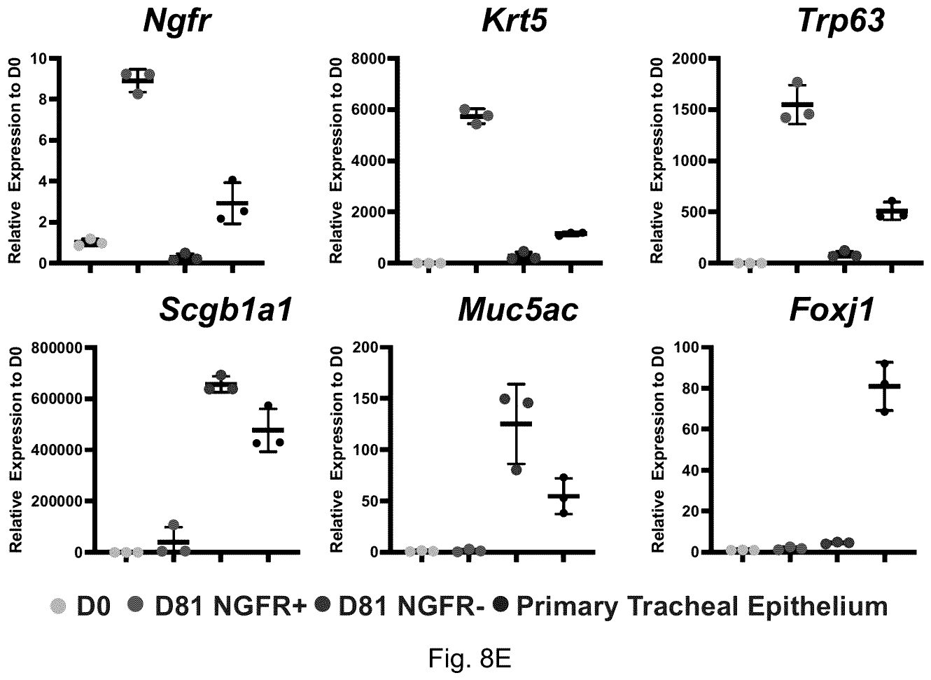

To develop an approach for reconstituting the resident stem cell compartment of the murine trachea, a common serum-free, feeder-free medium was developed to allow both primary mouse BCs and iBCs to be expanded in cell cultures into the large cell numbers for transplantation. Methods were used for derivation of a diversity of lung epithelial lineages including human iBCs, and conditions were optimized for similarly differentiating mouse PSCs into iBCs (see e.g., FIG. 1A). Mycoplasma-free, karyotypically normal (see e.g., FIG. 8A) mouse PSCs carrying an Nkx2-1^{mCherry} reporter were first differentiated into definitive endoderm, patterned the endoderm into anterior foregut, lung specification was induced into Nkx2-1^{mCherry}+ primordial progenitors, and proximalized airway epithelial cells were generated in 28 days. Nkx2-1^{mCherry}+ cells were then purified at this stage for 3D-culture in MATRIGEL in a “Basal Cell Medium” (here after “BCM”), composed of an FGF-supplemented airway media with added inhibitors of SMAD-dependent BMP and TGF-β signaling. Exposure of PSC-derived airway organoids to BCM induced upregulation of the basal cell surface protein marker, NGFR (see e.g., FIG. 1B-1D). By day 40-44, Nkx2-1^{mcherry}+ monolayered epithelial spheres emerged, with 54.73+/−16.00% of cells co-expressing Nkx2-1^{mcherry} and NGFR (see e.g., FIG. 1B-1D). After single-cell dissociation, passaging, and further expansion as epithelial spheres in BCM, the percentage of Nkx2-1^{mcherry}+/NGFR+ cells increased to 73.86+/−9.42% (see e.g., FIG. 1D).

Comparing sorted Nkx2-1^{mcherry}+/NGFR+ cells and sorted Nkx2-1^{mcherry}+/NGFR-cells vs. fresh primary tracheal epithelium, expression levels of canonical basal cell markers, Krt5 and Trp63, in the NGFR+ population were at levels equal to or greater than primary tracheal epithelium, indicating their identity as bona fide iBCs. In contrast, the smaller population of NGFR− cells were depleted of basal cell markers and instead expressed airway secretory cell markers, Scgblal and Muc5ac (see e.g., FIG. 1E-1F). Minimal ciliated marker expression (Foxj1) was observed in either PSC-derived population. iBCs were serially passaged at least 10 times without losing their proliferation potential, generating more than 10¹⁵ cells per input cell over 9 passages (see e.g., FIG. 1G, see e.g., FIG. 8B). To assess the stability of canonical gene expression over time, sorted NGFR+ and NGFR− iBC cultures were analyzed at P1, P3, P5, P7 and P9, and maintained expression of canonical airway epithelial lineage genes, Nkx2-1, Krt5, and Trp63, was found in the progeny of NGFR+ cells; Nkx2-1, Scgb1a1 and Muc5ac was found in NGFR− cells (see e.g., FIG. 8C). After cryopreservation and subsequent thawing, iBCs exhibited karyotypic and outgrowth stability in BCM, and they maintained their Nkx2-1^{mCherry} and BC marker expression profiles, which are quality assurance measures for a freezer-archived source of cells for potential transplant therapies (see e.g., FIG. 8D-8E).

Next, the outgrowth of passaged iBCs was characterized after extensive serial passaging (P6) using scRNA-Seq. Again, heterogeneity was evident as a mixture of two airway lineages: a major basal-cell like population (Nkx2-1+, Trp63+, Krt5+) and a minor population of secretory-like cells (Nkx2-1+, Scgb1a1+, Muc5b+). There was little to no evidence of ciliated, alveolar, or non-lung fates emerging (see e.g., FIG. 1H-1I). The identity of the basal cell cluster was further confirmed based on the observation that canonical basal cell markers, Krt5, Krt14, Krt17, and Trp63, were all amongst the top 20 most differentially enriched transcripts in these cells (see e.g., FIG. 1J).

To ensure that identical culture conditions could be used to prepare primary BCs for transplantation, isolated primary EPCAM+ cells were next from mouse tracheas for culturing in BCM (see e.g., FIG. 1K). Outgrowth of cells expressing BC markers (see e.g., FIG. 1L-1O and FIG. 8C) was observed over 5 serial passages, and the kinetics of expression of canonical airway epithelial marker genes (Nkx2-1, Krt5, Trp63, MucSac, and Foxj1) was monitored in sorted NGFR+vs NGFR− cells over time. There was evidence of increasing expression of basal cell markers Krt5 and Trp63 only in the NGFR+ cell fraction (see e.g., FIG. 8C). The proliferation rate of primary BCs slowed at P5, in contrast to iBCs, and primary BC could not be expanded beyond P5 (see e.g., FIG. 1P). Taken together, these results indicated successful derivation of mouse iBCs from PSCs and development of a serum-free/feeder-free medium, BCM, that can be used to expand both primary BCs and iBCs in identical culture conditions for comparative transplantation studies.

### Airway Epithelial Reconstitution by Mouse Primary BC Transplantation

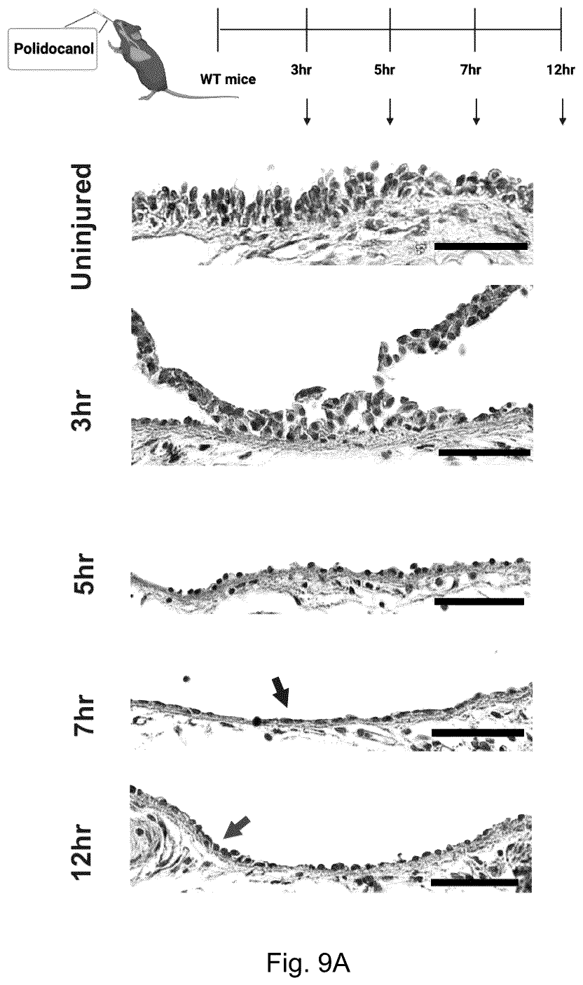

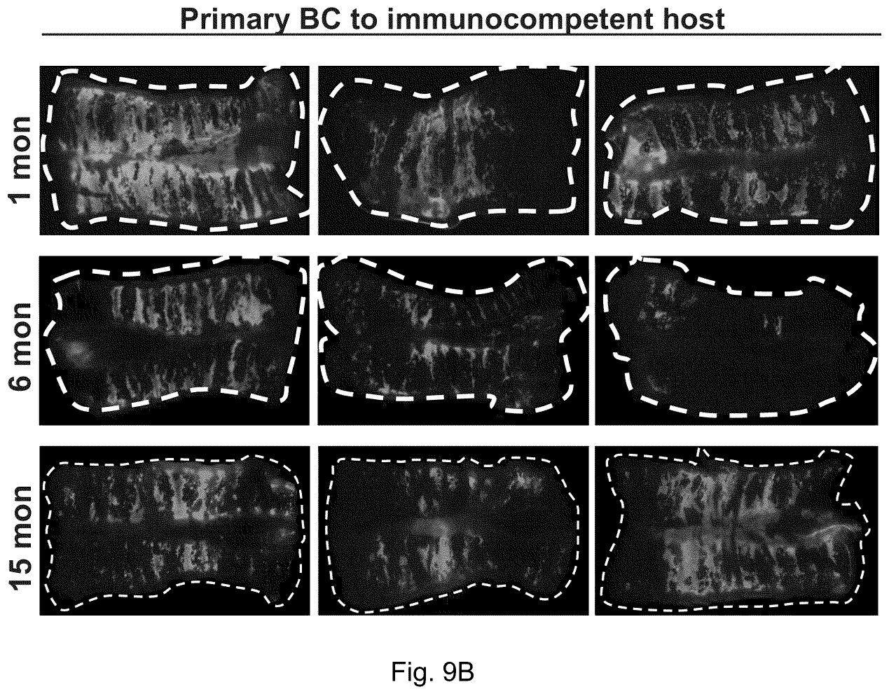

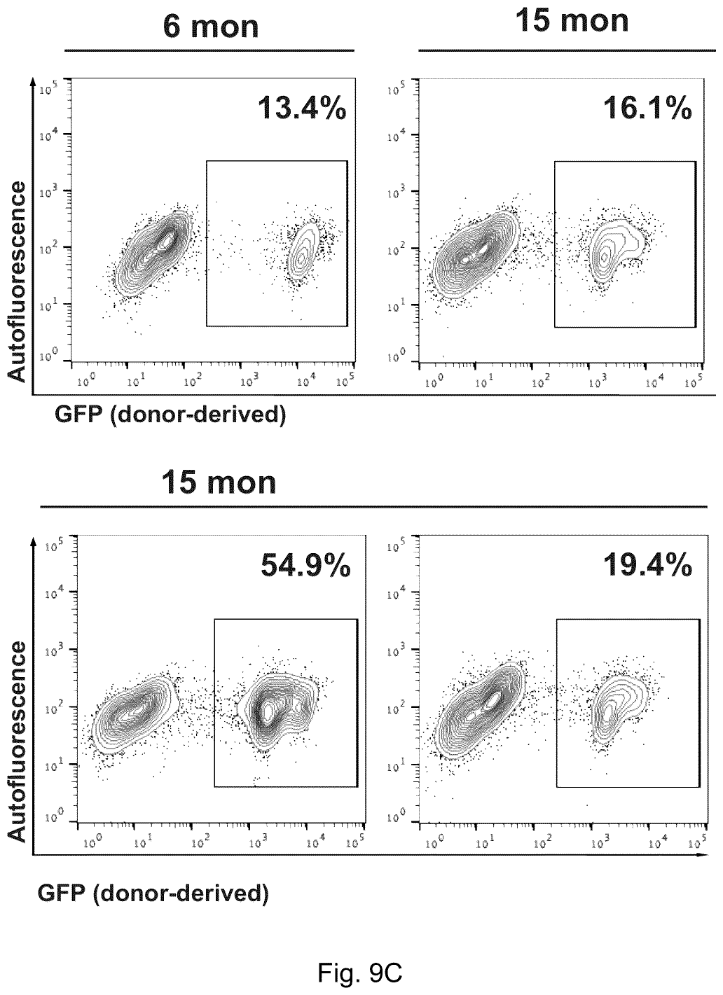

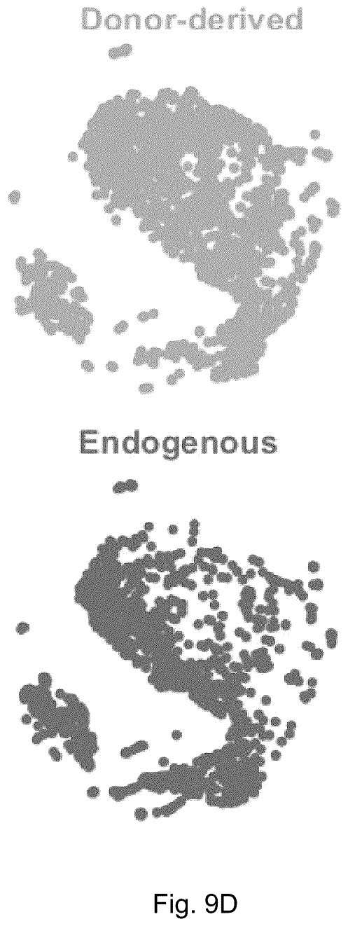

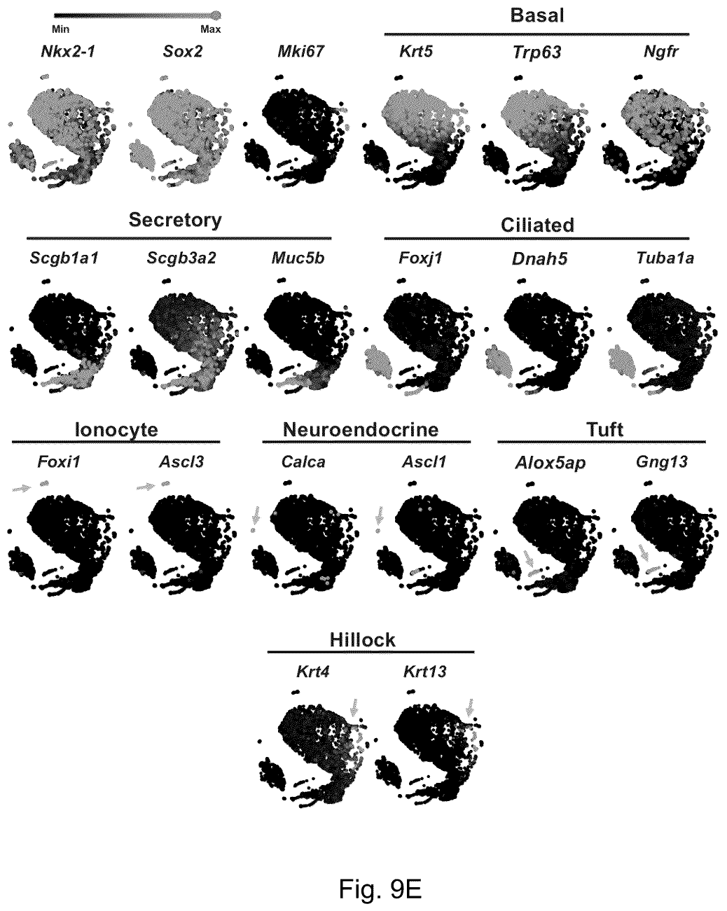

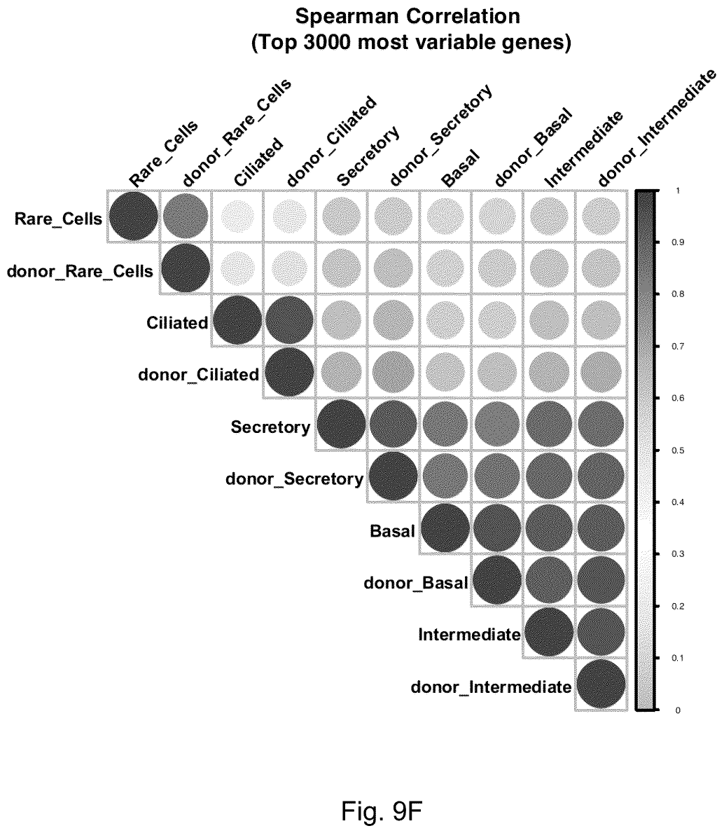

Intra-tracheal delivery of polidocanol was employed to ablate tracheal airway epithelial cells (see e.g., FIG. 9A). Tracheal epithelial sloughing occurs within three hours of polidocanol exposure, and by 24 hours surviving proliferating endogenous basal cells begin to cover this injured microenvironment; see e.g., Borthwick et al. (2001). Am J Resp Cell Mol 24, 662-670; Engler et al. (2020). Cell Reports 33, 108553; the contents of which are incorporated herein by reference in their entireties. It is contemplated herein that acute epithelial sloughing can generate a temporarily exposed niche on the airway basement membrane for exogenous cells to land. To determine the time window for BC transplantation post polidocanol-mediated injury, tracheal tissues were analyzed in a time series following injury. Extensive epithelial sloughing was confirmed at three hours after polidocanol exposure. At five hours, epithelial sloughing was more complete with sporadic residual individual cells and more denuded basement membrane. By seven hours, residual cells had flattened to cover the basement membrane, and by 12 hours patches of rounded or cuboidal epithelial cells characterized the regenerating epithelium (see e.g., FIG. 8A). Therefore, donor cells were transplanted five hours post-injury.

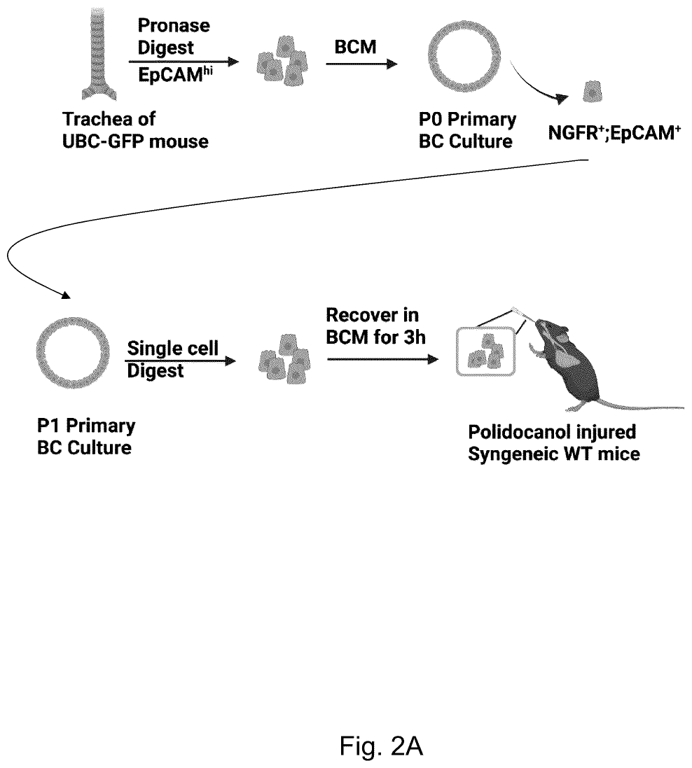

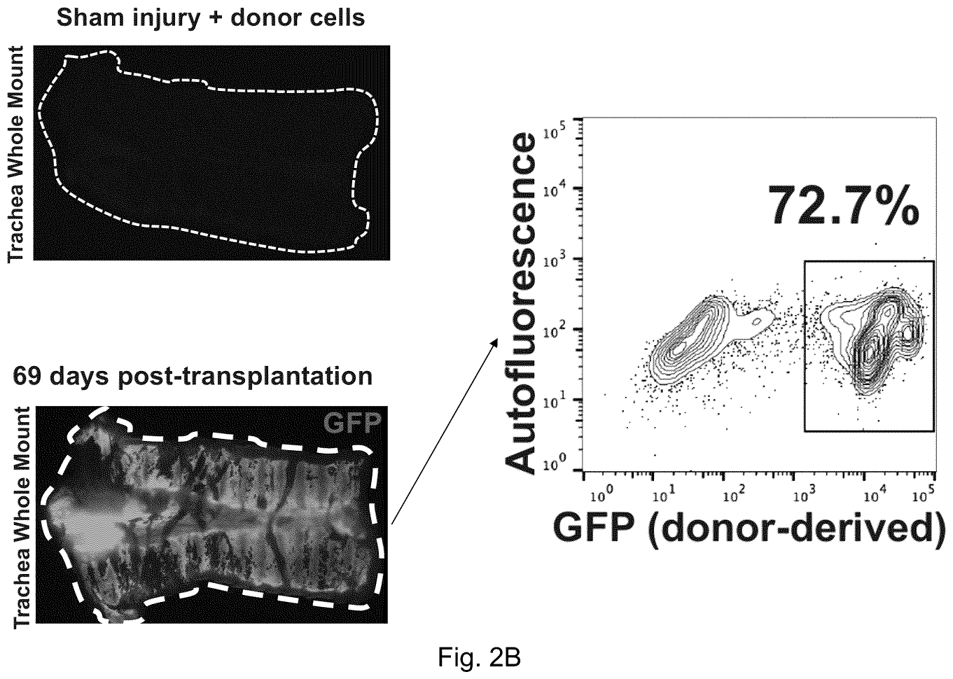

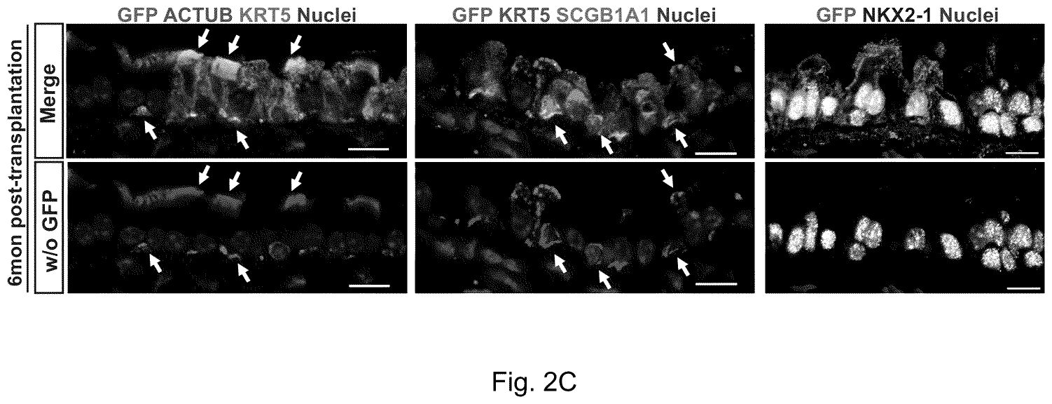

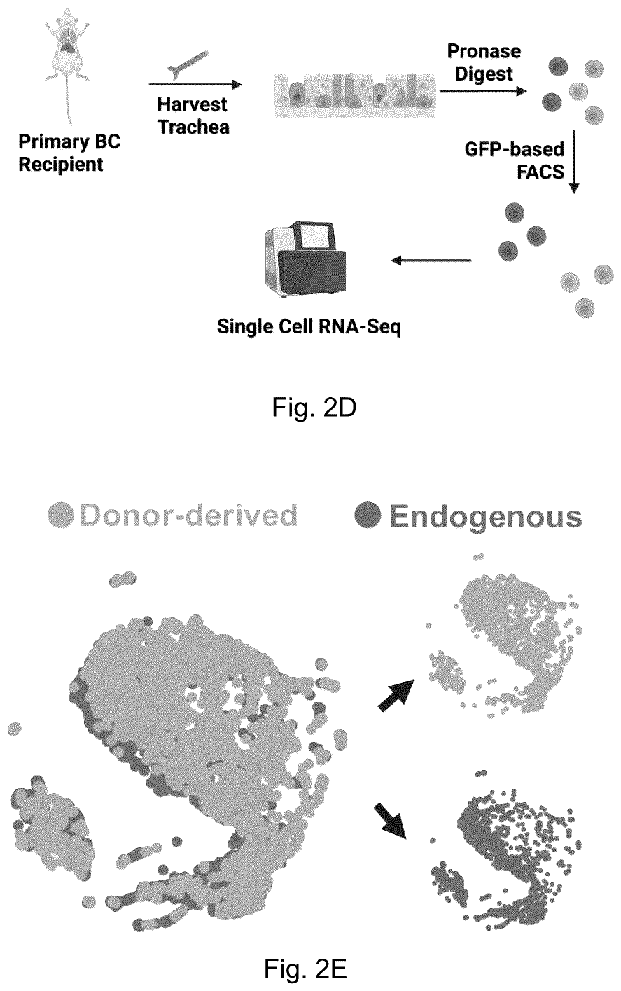

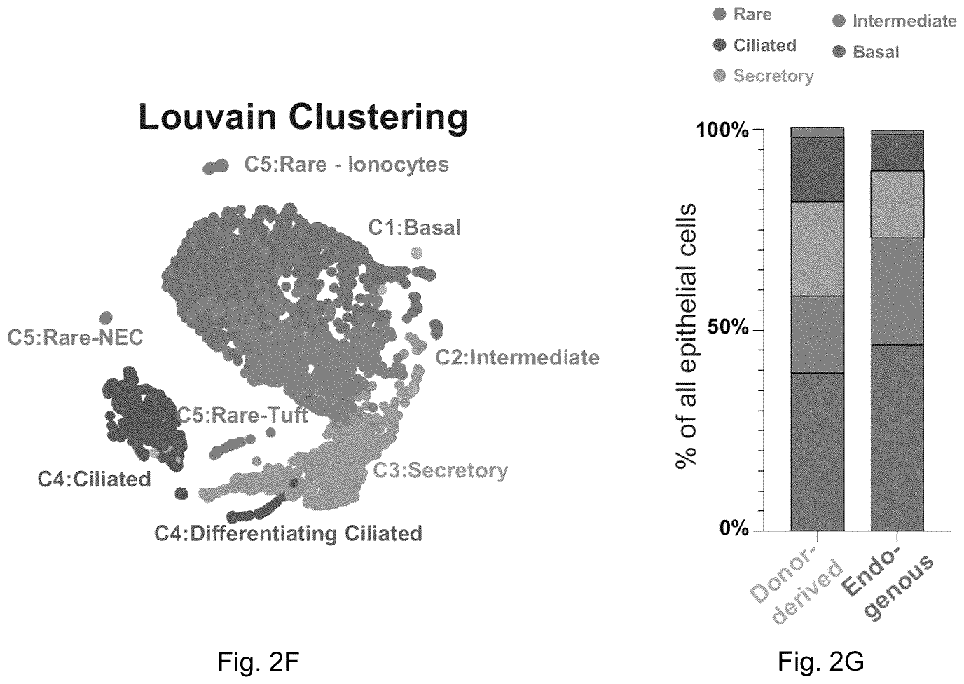

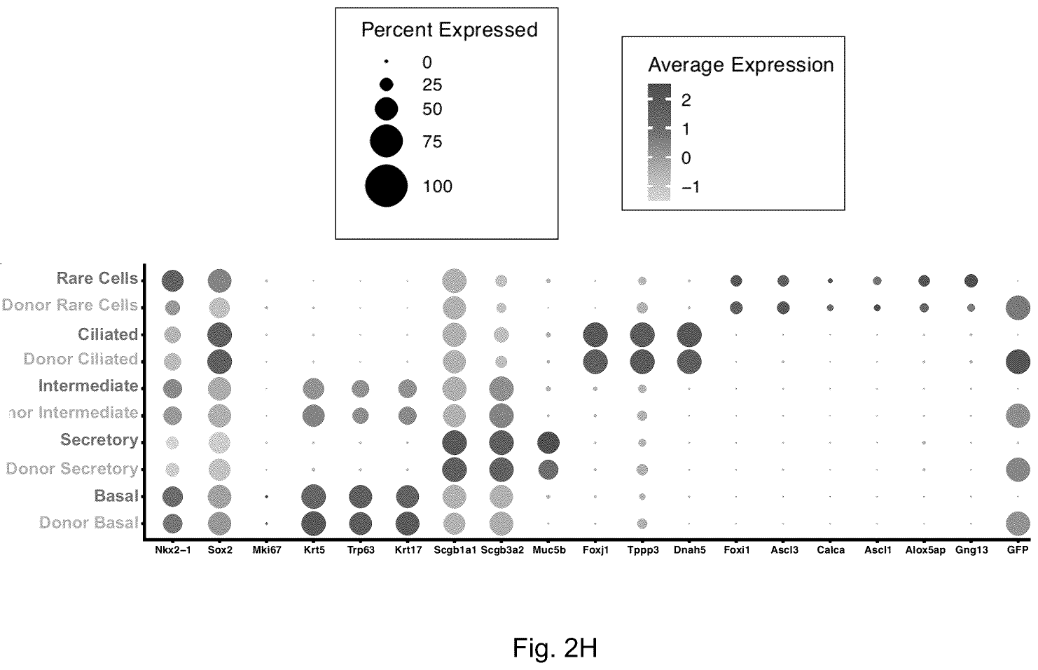

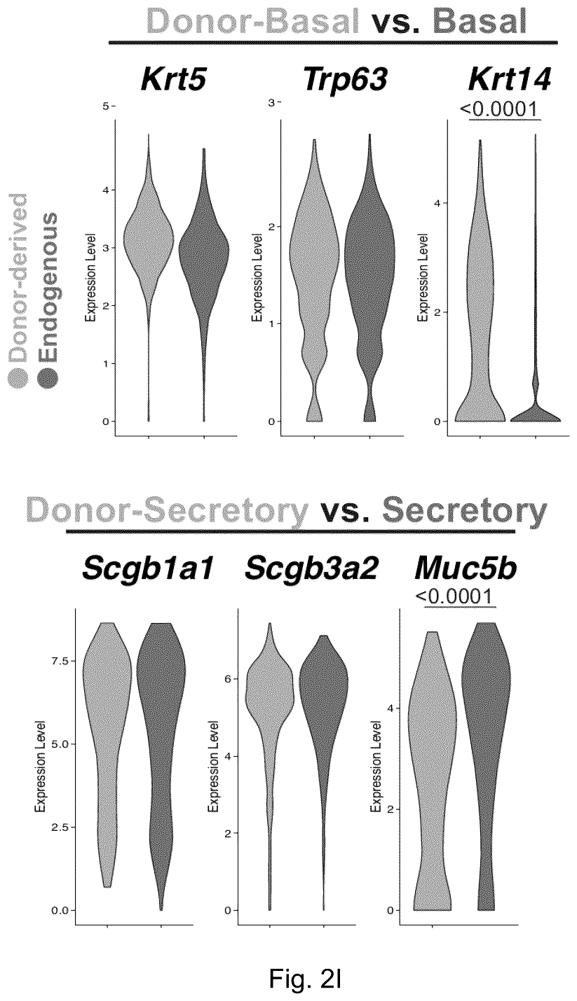

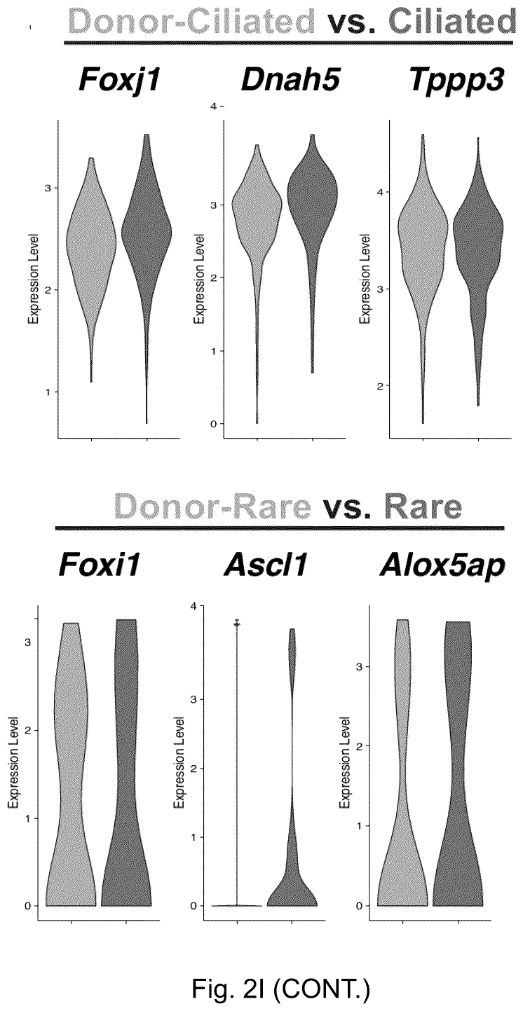

To test the feasibility of engrafting basal cells into the tracheal epithelium of injured recipients, cultured P1 primary BCs obtained from UBC-GFP donor animals were prepared, and 6*10⁶ cells were transplanted per animal into immunocompetent syngeneic mice five hours after polidocanol injury (see e.g., FIG. 2A). As expected, in uninjured recipients (PBS sham injury) no engraftment was observed of transplanted BCs (see e.g., FIG. 2B). In recipients analyzed at one month, two months, and six months post-transplantation, GFP+ donor-derived cells were observed retained in recipient injured tracheas. By epifluorescence, GFP+ cells contributed to an average of 31.89%+/−18.47% of recipient epithelium (N=10, see e.g., FIG. 2B, FIG. 9B-9C), and in some cases, by flow cytometry donor cells comprised as much as 74% of the recipient trachea, indicating significant reconstitution of the tracheal epithelium with donor-derived cells (see e.g., FIG. 2B). By immunofluorescence, donor cells were found localized to the tracheal epithelium, but not detectably to the submucosal glands; the donor cells expressed NKX2-1 nuclear protein, indicating retention of lung cell fate. Donor-derived cells co-expressed GFP along with markers of tri-lineage differentiation into airway basal (keratin 5, KRT5+), secretory (Secretoglobin Family 1A Member 1, SCGB1A1+), or ciliated (acetylated tubulin, ACTUB+) fates (see e.g., FIG. 2C).

To further assess the molecular phenotypes of the progeny of transplanted BCs and compare them to their endogenous counterparts in the same recipient, scRNA-Seq was performed on 1807 GFP+(donor-derived) and 1294 GFP− (endogenous) epithelial cells from a recipient trachea 69 days post-transplantation. GFP+ and GFP− cells were captured and sequenced simultaneously to avoid batch effects (see e.g., FIG. 2D). The global transcriptomes of donor-derived and endogenous cells were highly similar (see e.g., FIG. 2E, dimensionality reduction visualization of transcriptomes by SPRING). Louvain clustering analysis identified five cell clusters (see e.g., FIG. 2F), distinguished based on expression of known canonical markers of airway epithelial cell types. Donor-derived cells contributed to all major (basal, secretory, and ciliated) and minor (neuroendocrine, tuft, Hillock, and ionocyte) airway epithelial cell types (see e.g., FIG. 2F-2H, FIG. 9D). Both donor-derived and endogenous cells were similarly quiescent, and no alveolar or non-lung fates were detected (see e.g., FIG. 2H, FIG. 9E). Donor-derived cells were highly similar to their endogenous counterparts, as evidenced by high Spearman correlation coefficients for each donor vs endogenous cell lineage (see e.g., FIG. 2H, FIG. 9F). For each airway cell type, pairwise comparisons of GFP+vs GFP− cells indicated that less than 40 genes were differentially expressed. There was no statistically significant difference in canonical lineage marker expression between GFP+vs. GFP− cells, with the exceptions of Krt14 for basal cells (upregulated in donor-derived) and Scgb3a1 and Muc5b for secretory cells (downregulated in donor-derived, see e.g., FIG. 2I). Together, these results demonstrate the ability to engraft cultured basal cells into immunocompetent recipient mouse tracheas after polidocanol-induced epithelial injury.

### Airway Epithelial Reconstitution by Mouse iBC Transplantation

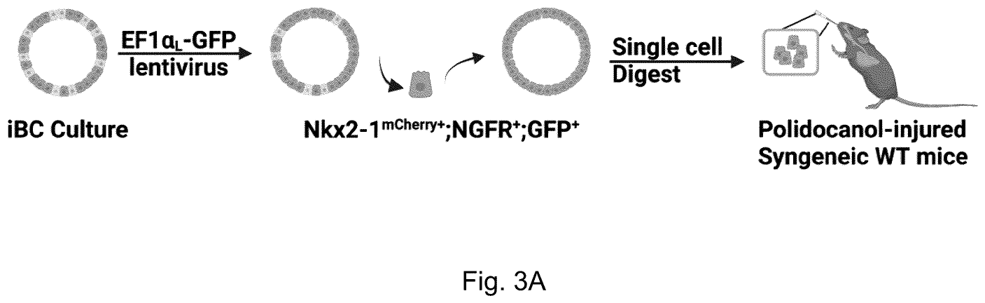

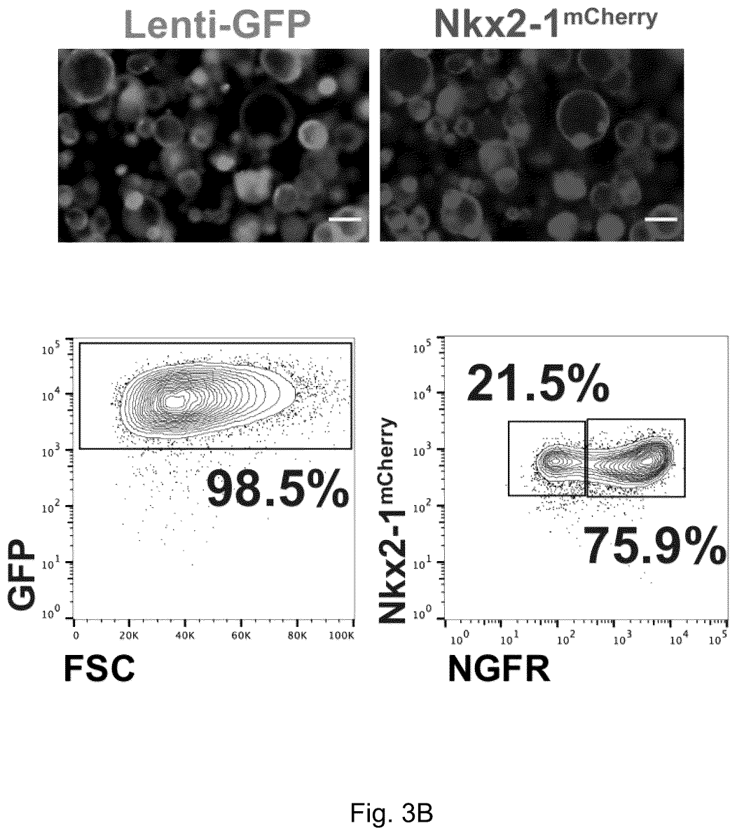

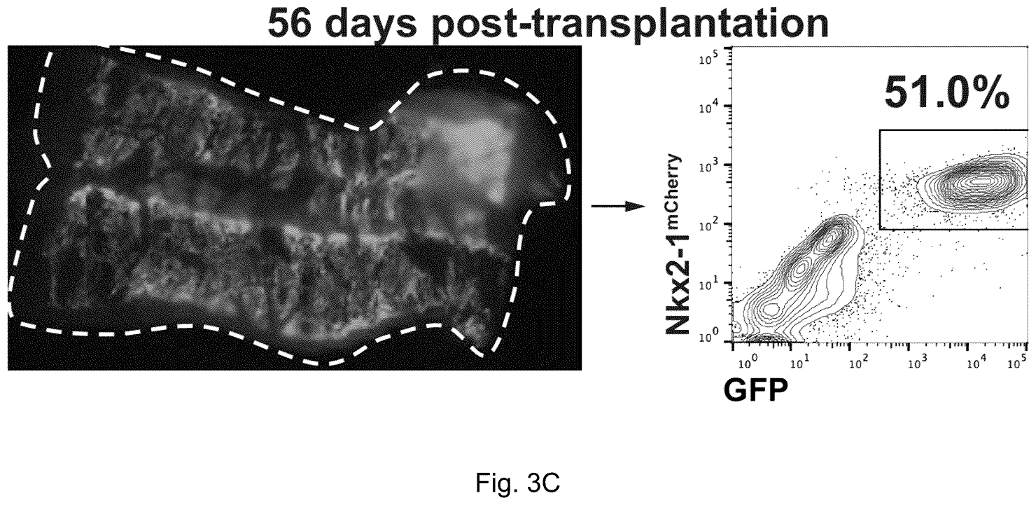

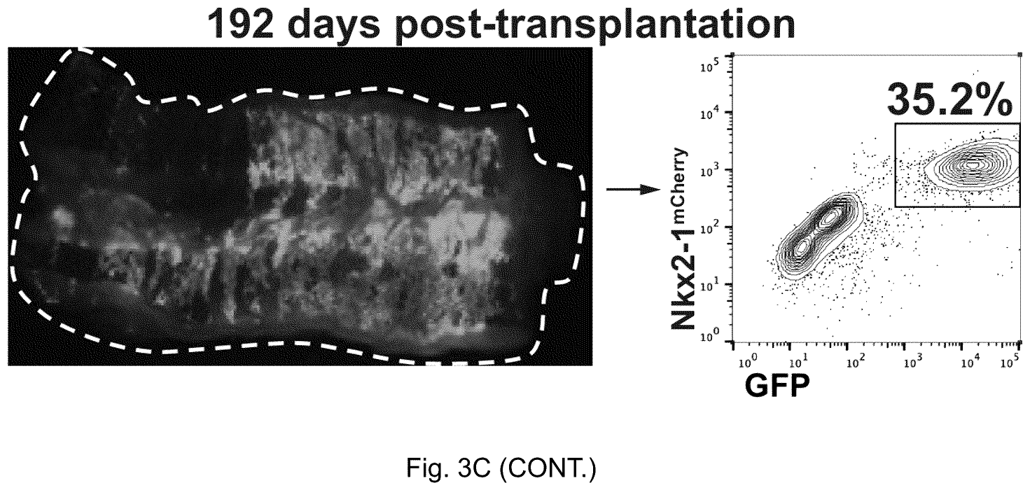

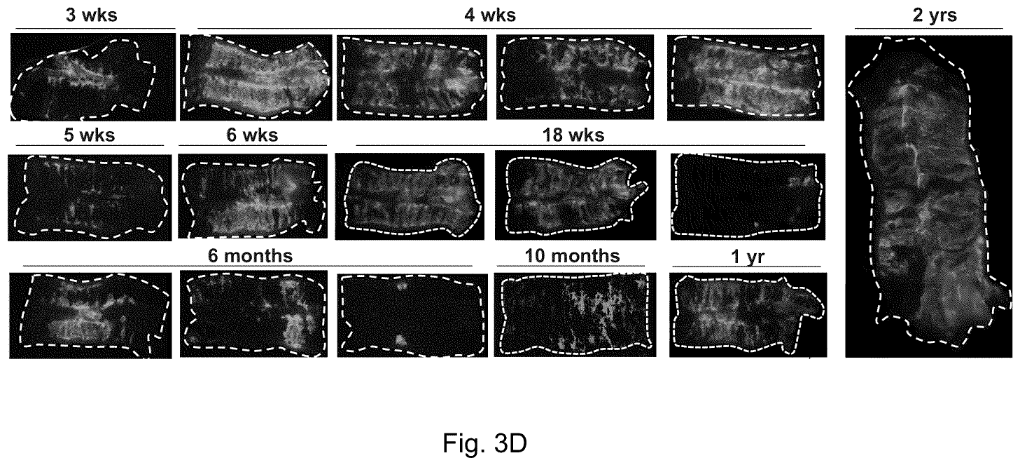

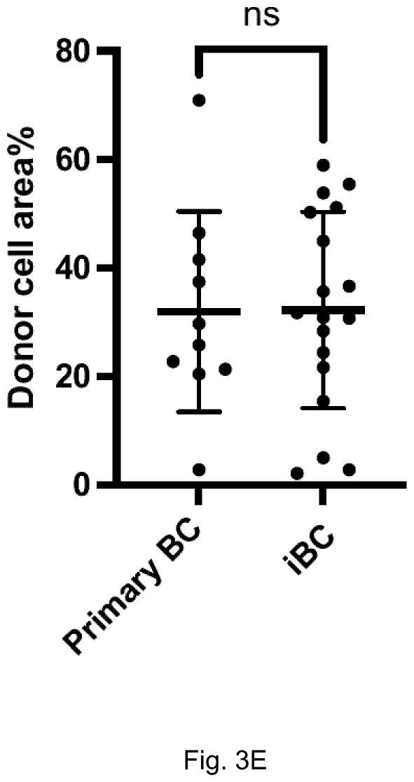

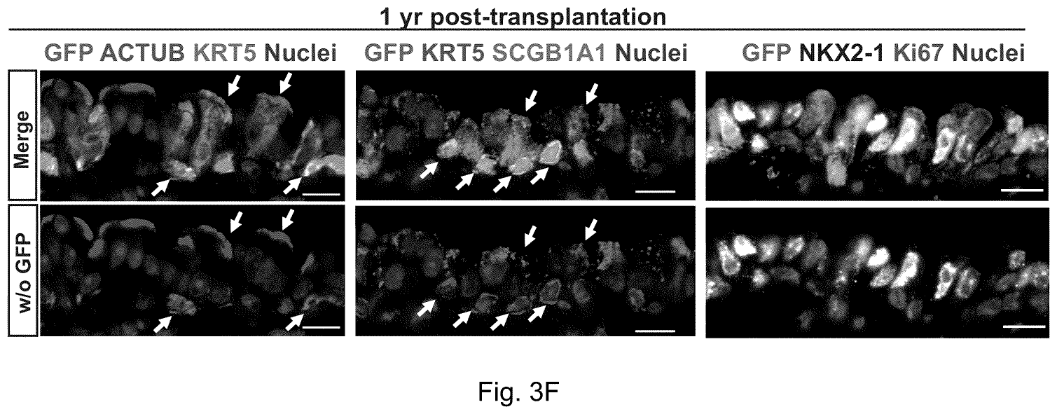

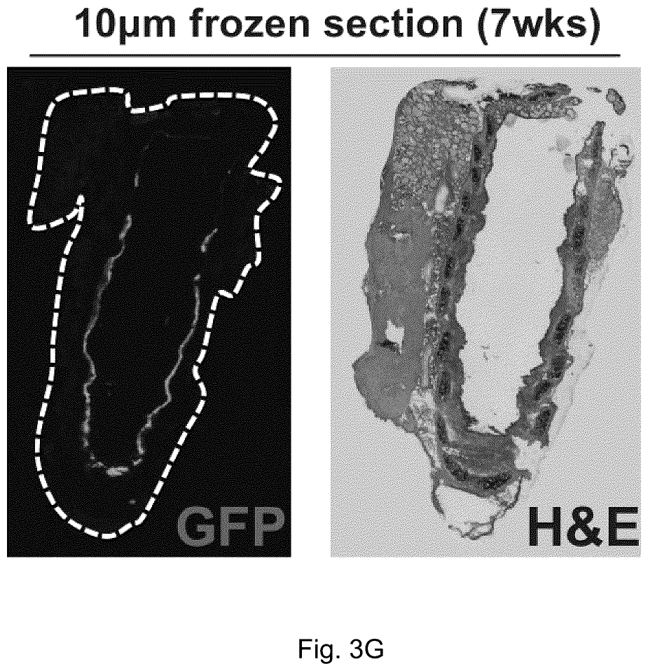

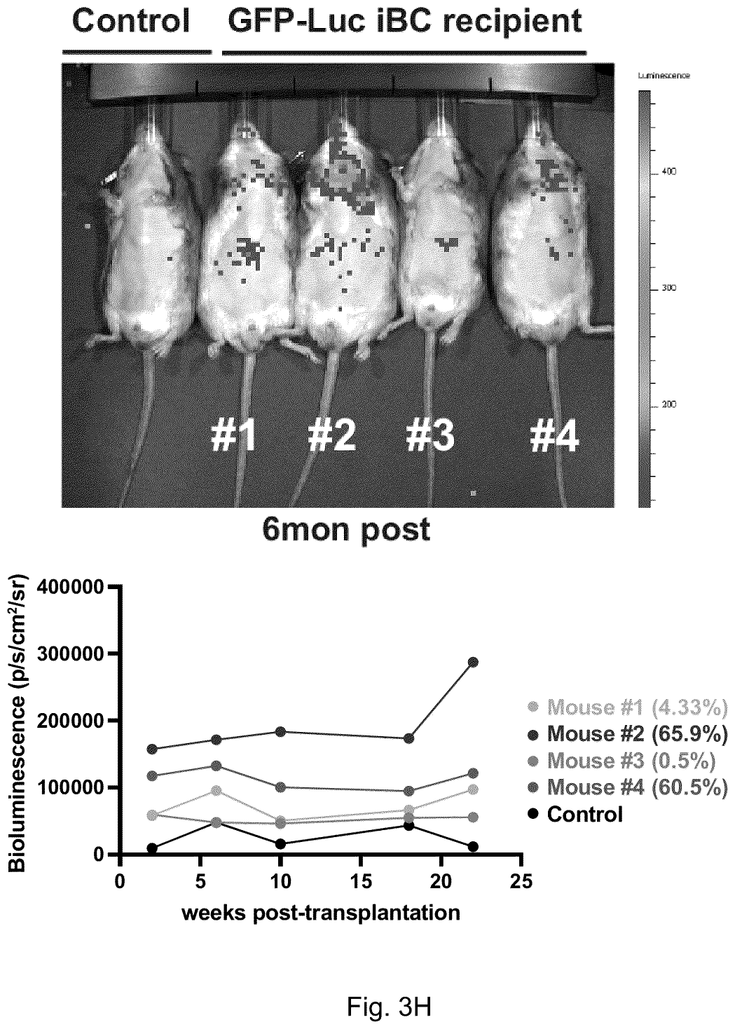

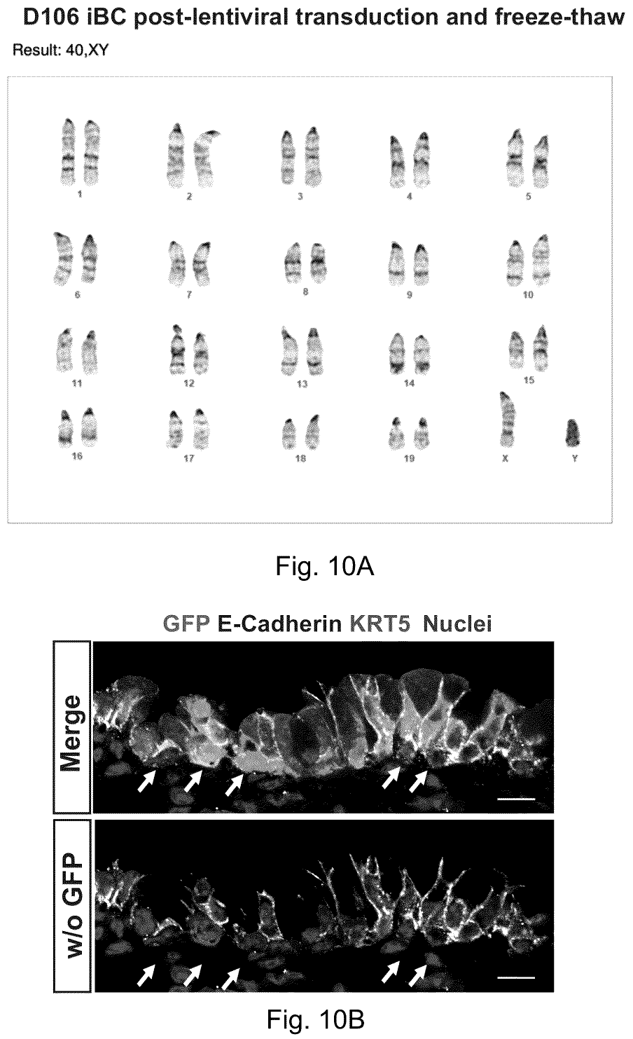

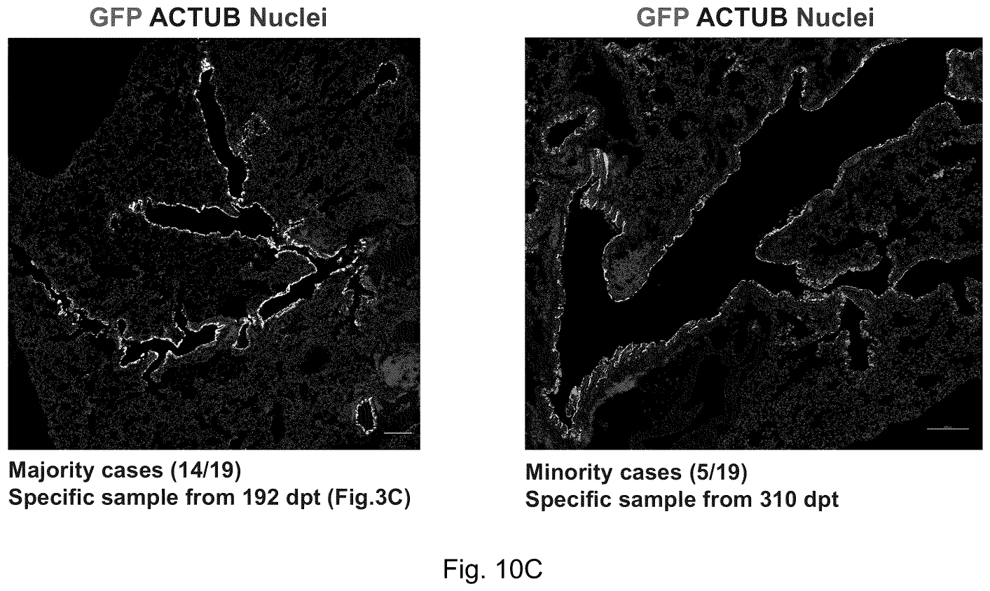

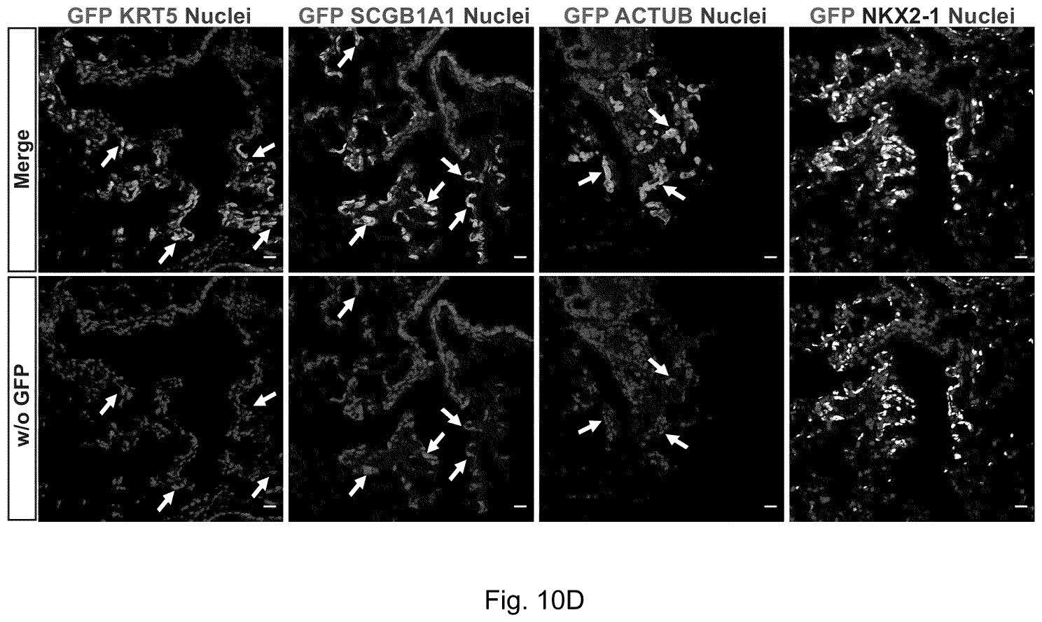

Having established an approach to deliver basal cells into the injured mouse trachea, it was next sought to test the ability of cultured iBCs to engraft in the stem cell compartment of immunocompetent mouse tracheas. Established cultures of iBCs carrying the Nkx2-1^{mCherry} reporter were labeled with a constitutively GFP-expressing lentivirus and subsequently sorted based on GFP+; Nkx2-1^{mCherry}+; NGFR+ expression to purify GFP-tagged iBCs for further culture expansion or cryopreservation. Following an additional 10-14 days of 3D culture, karyotypically normal (see e.g., FIG. 10A) cells were delivered intratracheally (without further sorting) into syngeneic immunocompetent recipients post-polidocanol injury (see e.g., FIG. 3A-3B). In recipients analyzed three weeks to two-years post-transplantation (N=19 total), donor-derived cells were easily detected based on tracheal whole mount microscopy (see e.g., FIG. 3C-3D). By epifluorescence quantification, GFP+ cells contributed to an average of 32.19+/−18.13% of iBC recipients' tracheal epithelium, similar to the efficiency of primary BC transplants (see e.g., FIG. 3E). In some cases, donor-derived cells comprised more than 50% of the epithelial cells of recipient tracheas by flow cytometry quantitation (see e.g., FIG. 3C). Immunofluorescence studies demonstrated that GFP+ cells retained lung fate (NKX2-1+) and contributed to basal (KRT5+), secretory (SCGB1A1+), and ciliated (ACTUB+) lineages in recipients. E-cadherin immunostaining indicated the expected apical-basal epithelial polarization of GFP+ cells and GFP+ basal cells were located on the basal epithelial surface next to the basement membrane (see e.g., FIG. 3F, FIG. 10B). GFP+ cells were not detected outside of the epithelial layer or in the submucosal glands (see e.g., FIG. 3G). The majority of recipients exhibited GFP+ cells in the extrapulmonary airways, with a minor fraction extending to intrapulmonary large and small airways (see e.g., FIG. 10C). When donor-derived cells were observed in the intrapulmonary airways, these cells appeared as basal, secretory, and ciliated lineages (see e.g., FIG. 10D).

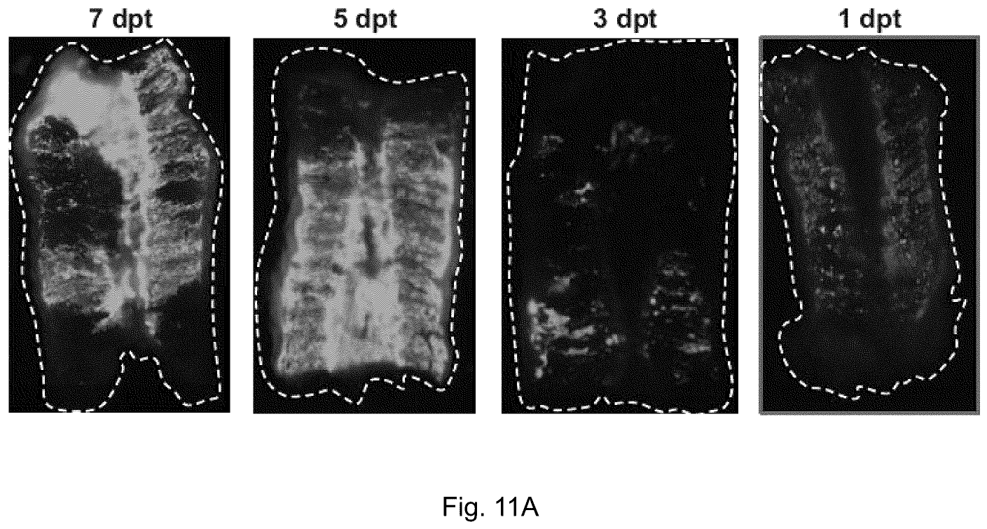

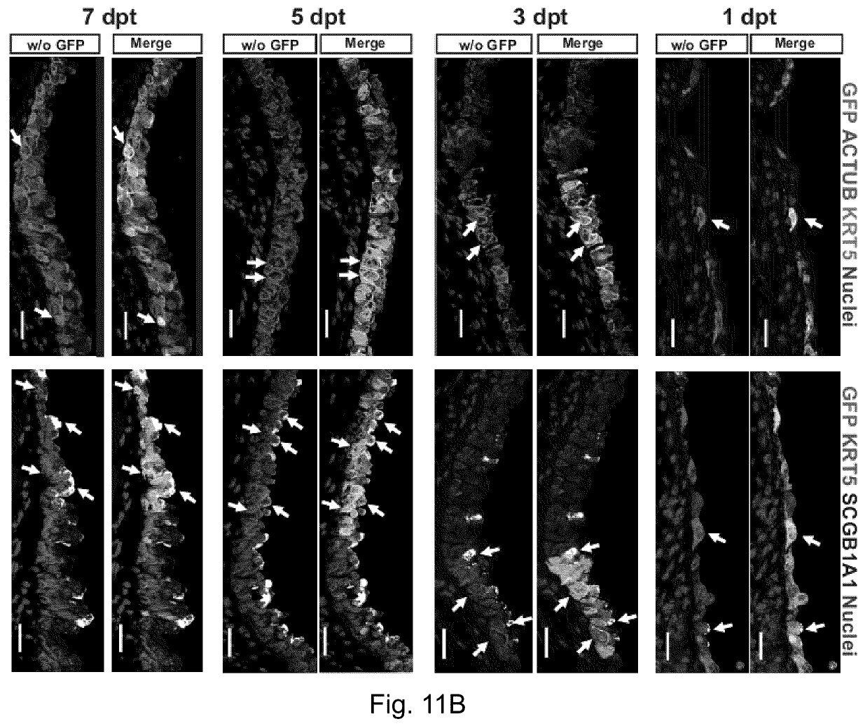

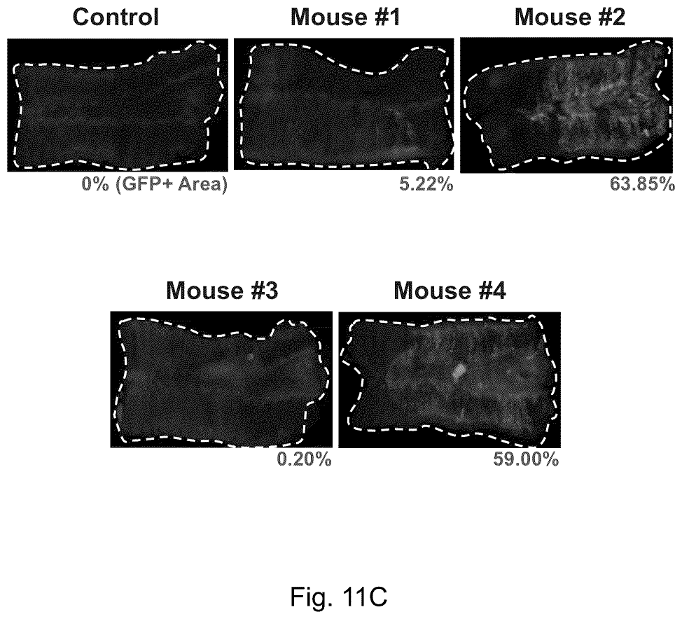

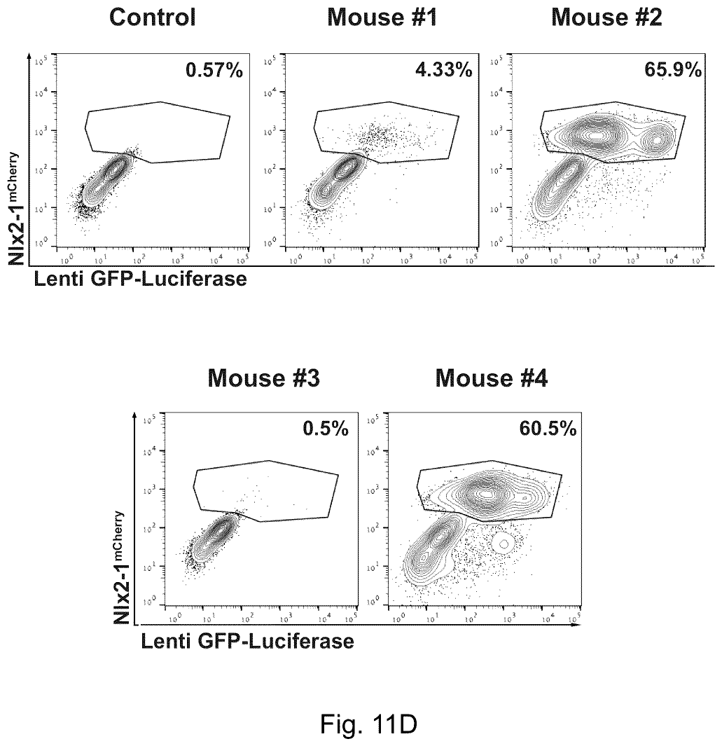

Next, the dynamics of engraftment was studied. Two approaches were taken: a) to study initial seeding and outgrowth of transplanted cells, recipients of GFP-tagged iBCs were harvested beginning on the first day post-transplant (dpt) and every two days thereafter for the first week, and b) iBCs were tagged with a luciferase and GFP-expressing lentivirus prior to transplantation, allowing each recipient to be followed longitudinally using intravital bioluminescence imaging. Initially (1 day post transplant (dpt)), donor-derived basal-like cells (GFP+; KRT5+) represented the majority of the cells, attaching to the basement membrane as single cells and assuming a flattened morphology covering the tracheal basement membrane at sites of injury. By 3 dpt, donor-derived cells formed small patches of KRT5+ cells, with cuboidal secretory differentiation evident by SCGB1A1 immunostaining. At 5 dpt, a reconstituted pseudostratified epithelium arose from contributions of both transplanted and endogenous cells, which was further established by 7 dpt (see e.g., FIG. 11A-11B). In recipients of luciferase-labeled iBCs (N=4), followed serially for 22 weeks after transplantation, intravital bioluminescence from transplanted cells was found only in the trachea or lung regions and was stable over time (see e.g., FIG. 3H), with bioluminescence measures of donor cell presence verified by both GFP fluorescence microscopy and flow cytometry at the time of harvest (see e.g., FIG. 11C-11D). No extrapulmonary bioluminescence signal was observed in any recipient (see e.g., FIG. 3H). Distal lung bioluminescence detected in one of four recipients indicated intrapulmonary engraftment, again consistent with engraftment of donor cells in the intrapulmonary airways of a subset of recipients (see e.g., FIG. 10C-10D).

scRNA-Seq of iBC-Derived Cells In Vivo

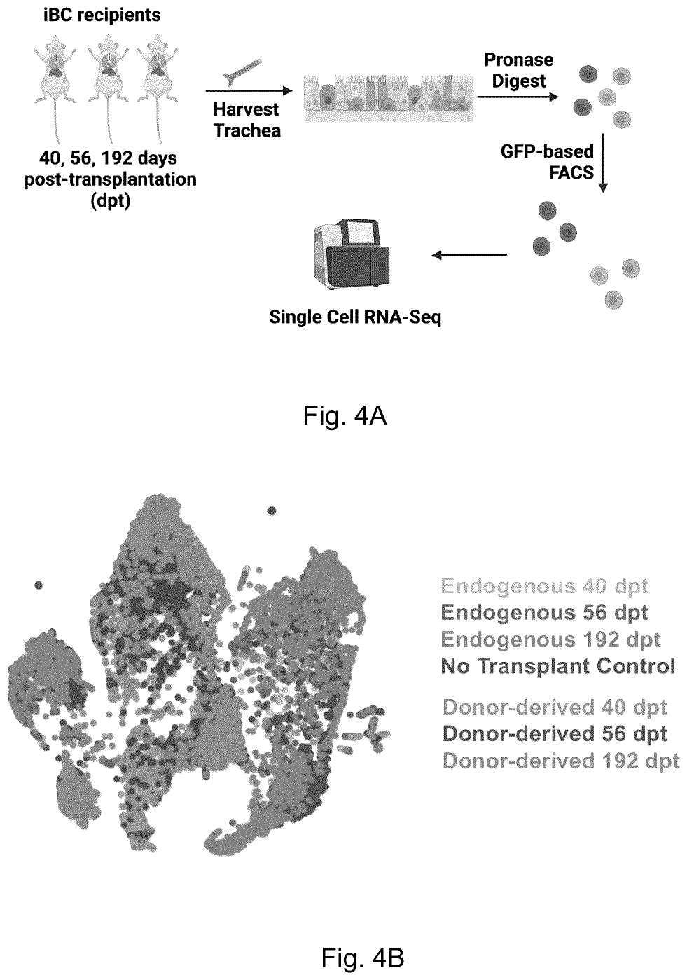

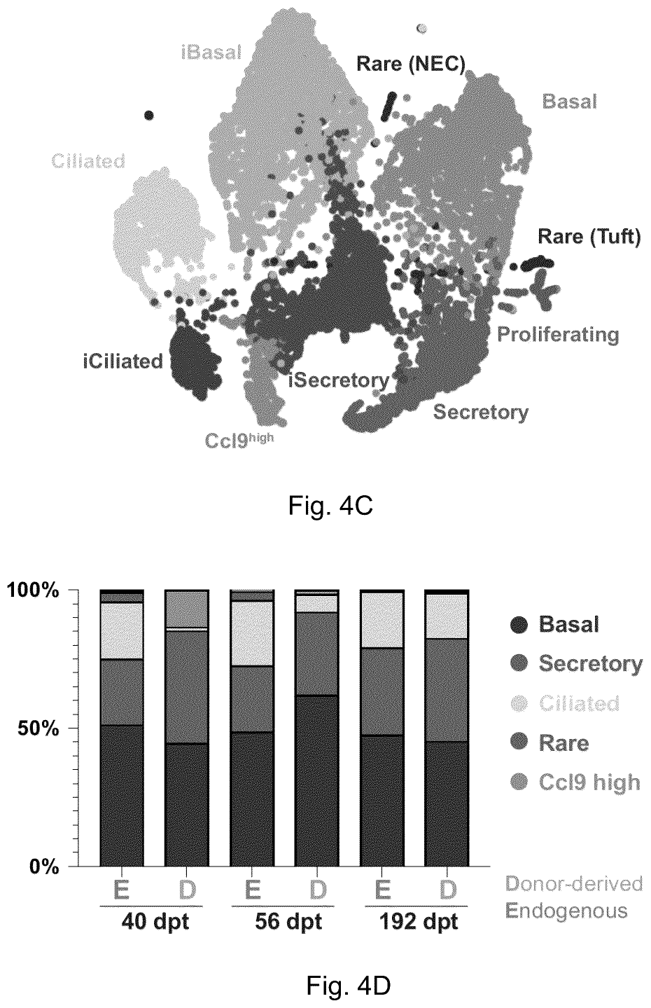

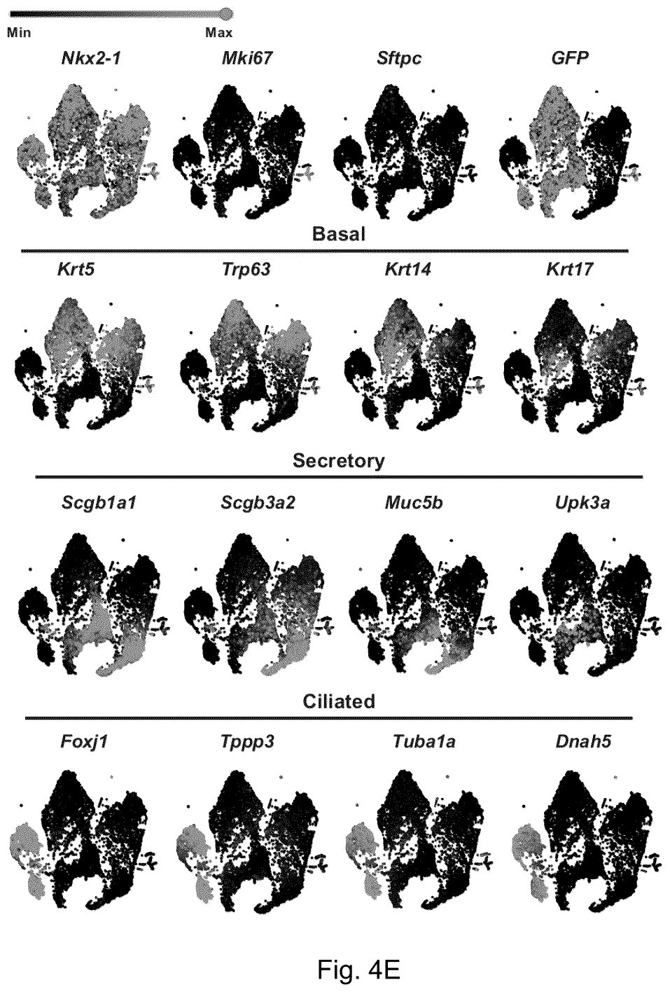

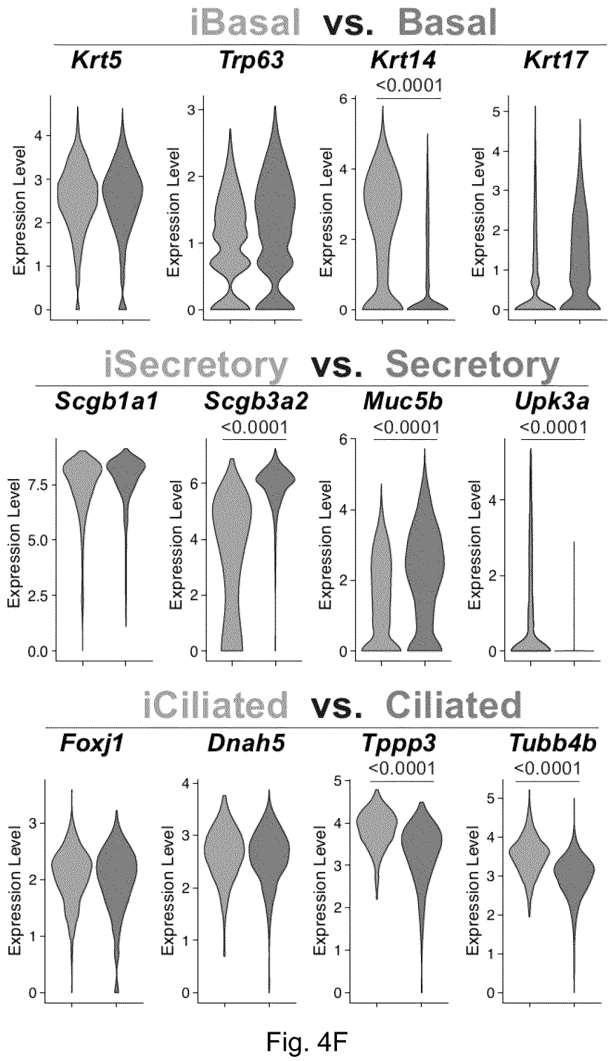

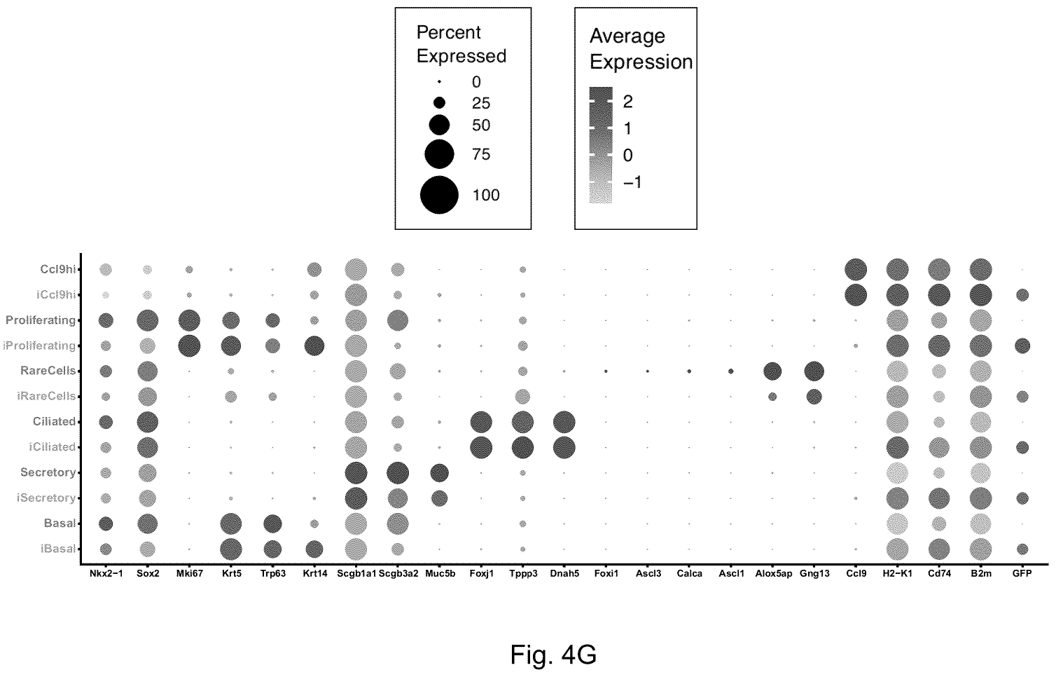

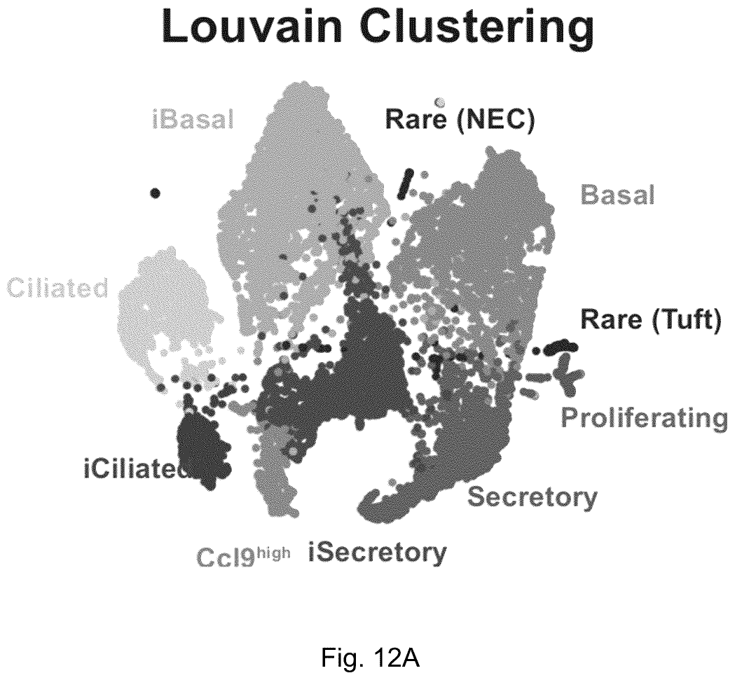

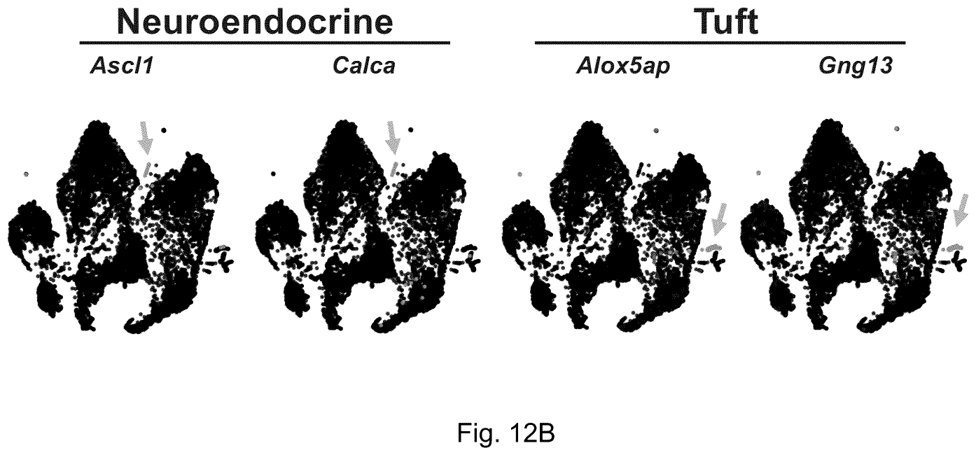

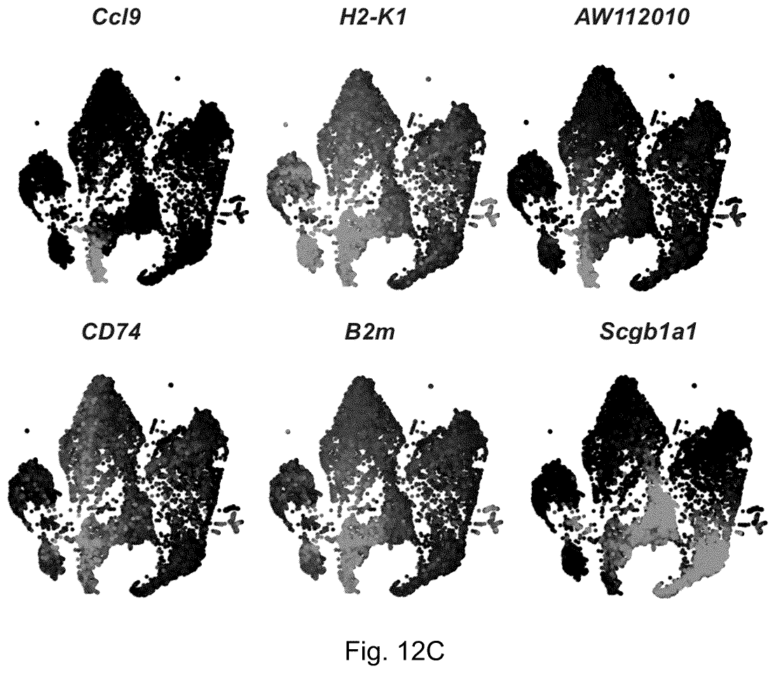

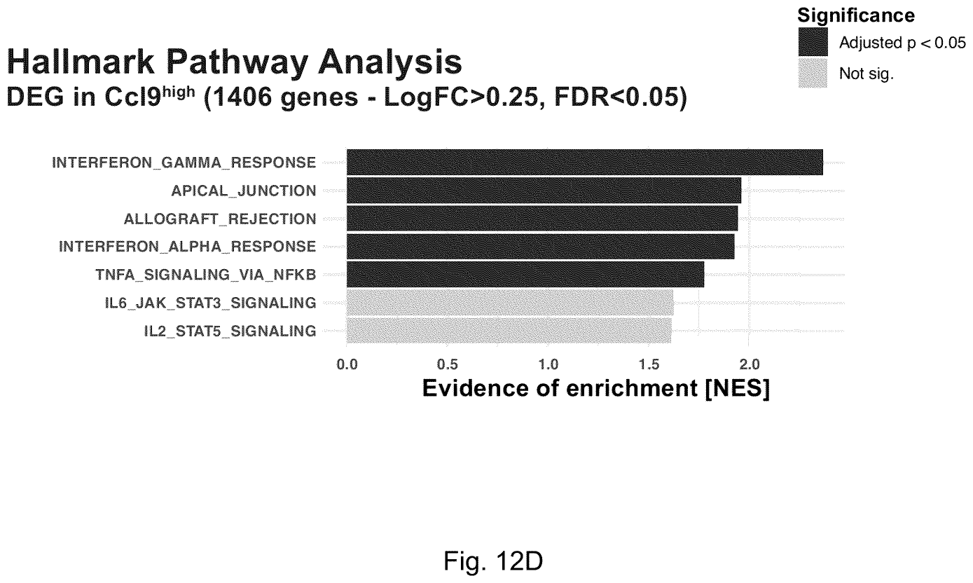

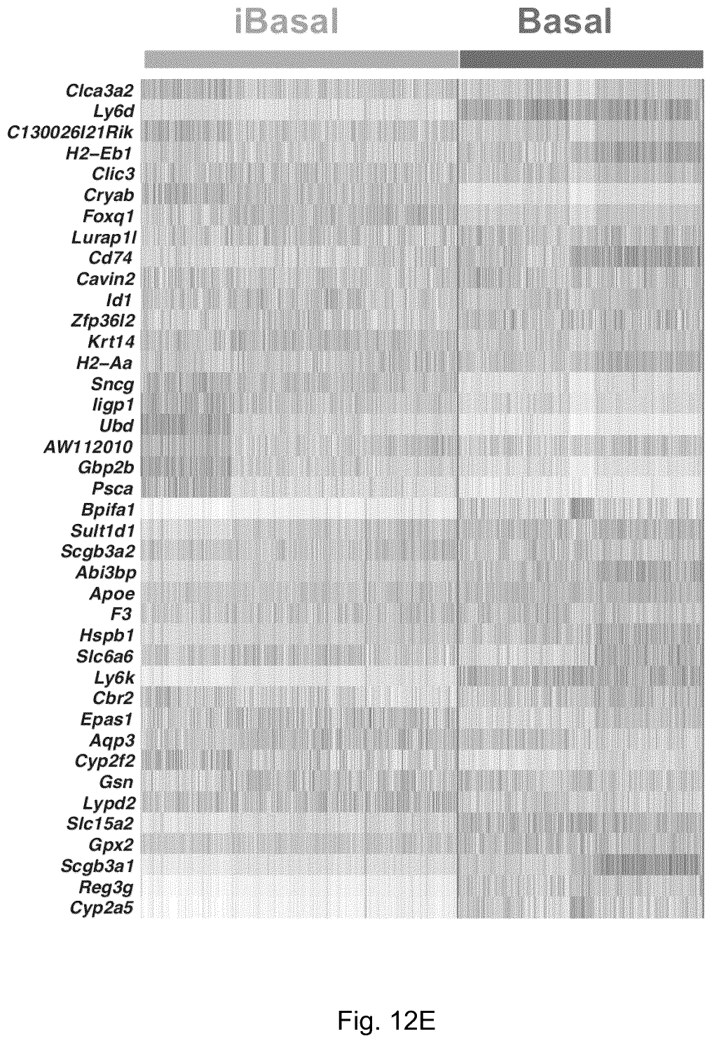

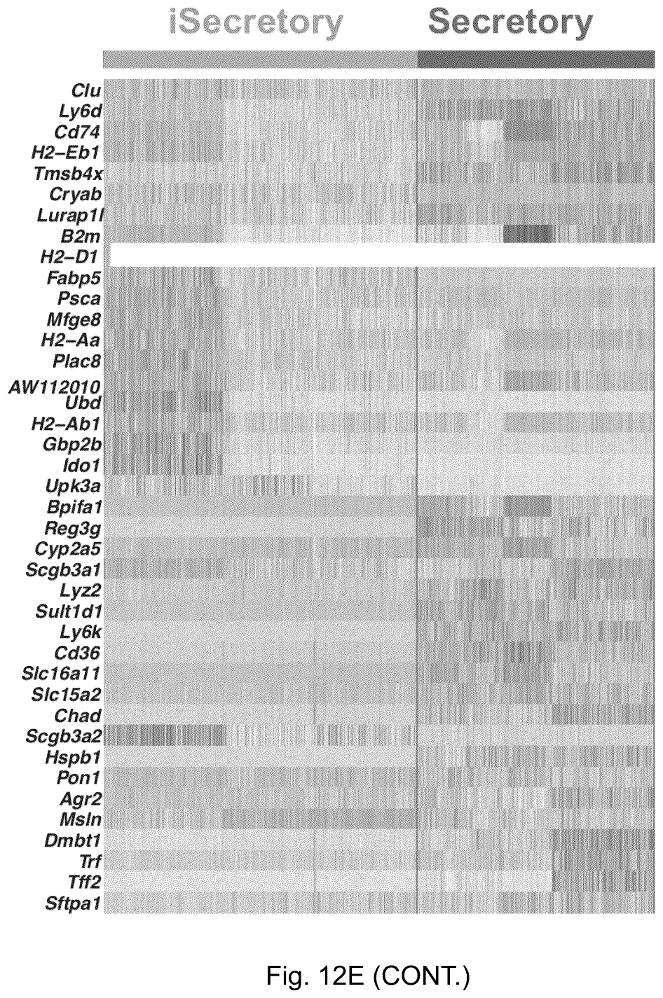

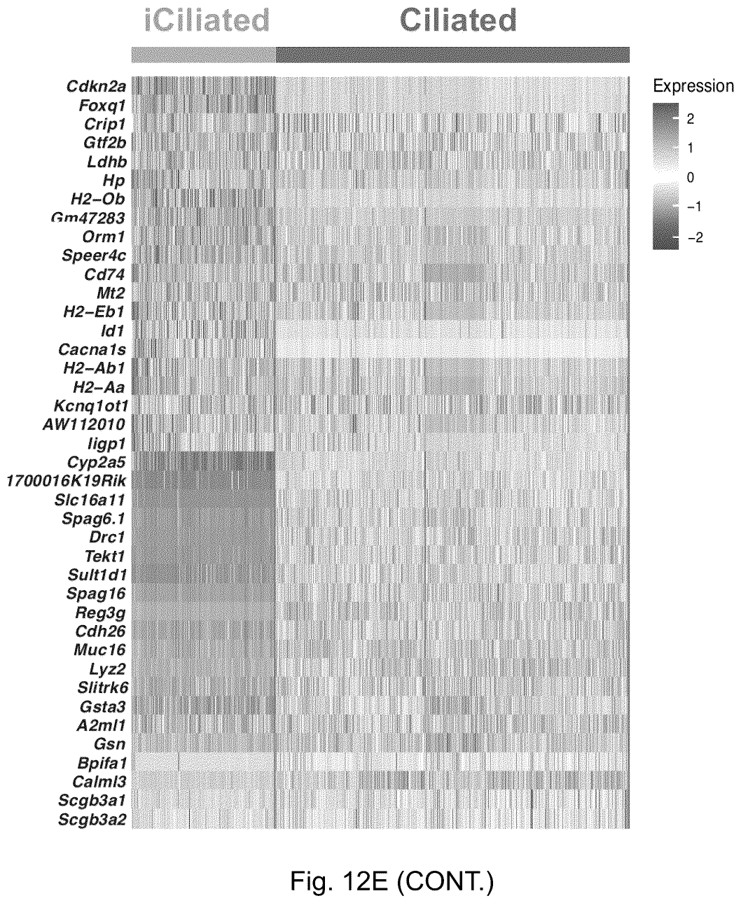

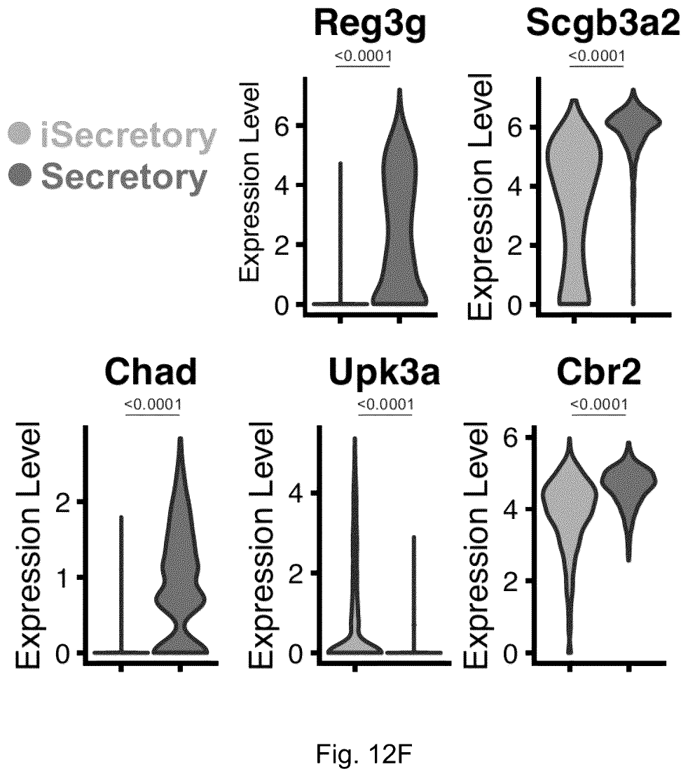

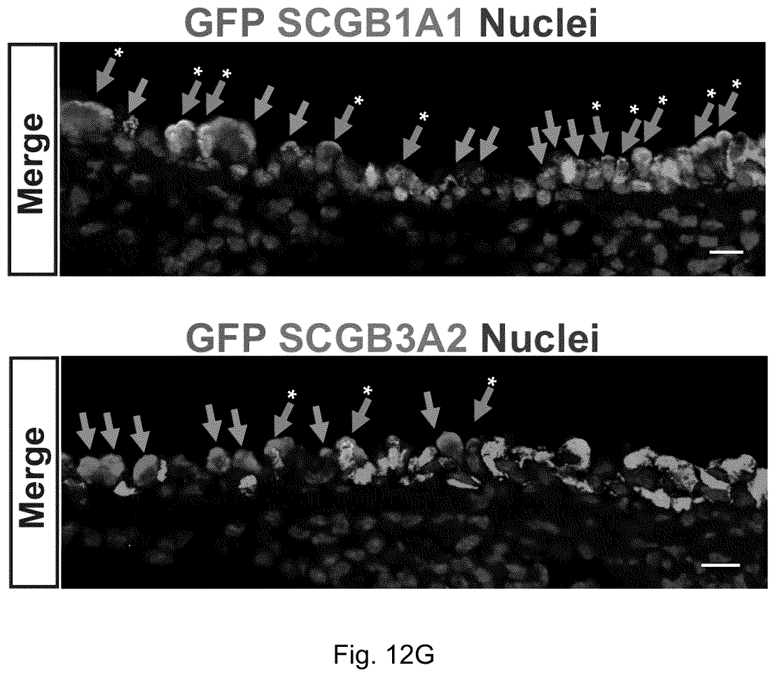

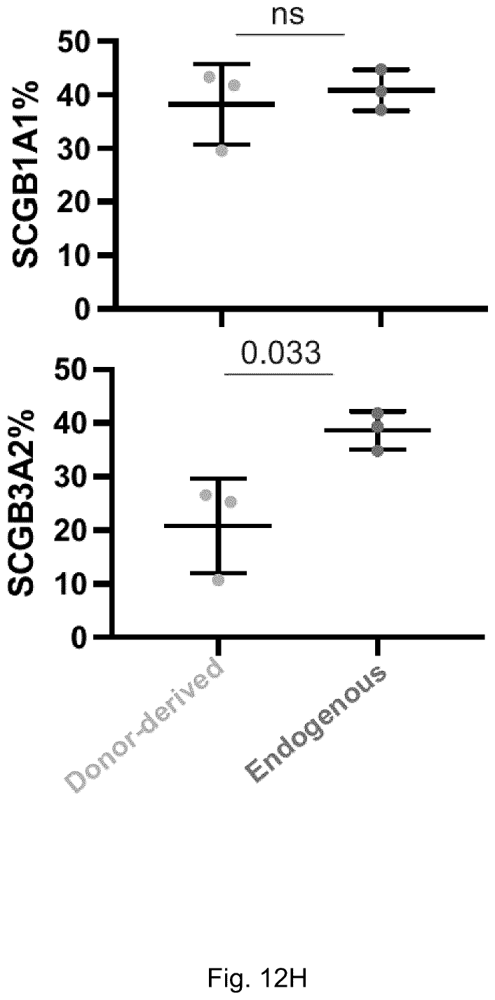

To assess the molecular phenotypes of transplanted iBCs compared to their endogenous counterparts, scRNA-Seq was performed of three recipient tracheas after prolonged recovery post-transplantation (40 dpt, 56 dpt, and 192 dpt), as well as a control trachea that received no injury and no transplantation (see e.g., FIG. 4A). Endogenous cells (GFP−) from each recipient, representing each of the 3 major tracheal epithelial cell lineages (basal, secretory, and ciliated), were evident on dimensionality reduction visualization and expressed global transcriptomes that closely overlapped with uninjured control mouse epithelium, indicating that transplanted iBCs did not significantly perturb the transcriptomes of endogenous cells. 1500-1700 GFP+ cells analyzed per recipient expressed Nkx2-1 and appeared as trilineage differentiated airway populations, each defined based on high levels of expression of canonical lineage-defining markers for basal (Krt5, Trp63, Krt14), secretory (Scgb1a1, Scgb3a2, Muc5b) and ciliated (Foxj1, Tppp3, Tubala) lineages. Louvain clustering of all cells identified nine cell clusters, with GFP+ cells clustering separately from endogenous cells for all three major airway subtypes (see e.g., FIG. 4C-4E). Both donor-derived and endogenous cells were similarly quiescent, and no alveolar or non-lung fates were detected (see e.g., FIG. 4E). Donor-derived rare airway cell types (neuroendocrine, tuft) were detectable as discrete clusters (see e.g., FIG. 12B), but ionocytes (endogenous or donor-derived) were too rare to be reliably identified.

Donor-derived ciliated cells were under-represented compared to endogenous cells in the two recipients harvested 40 dpt and 56 dpt, but the frequency was more similar in the recipient harvested at 192 dpt (see e.g., FIG. 4D). In addition, in 2/3 recipients, a distinct population of donor-derived Ccl9^{high} cells was detected, clustering adjacent to secretory club cells, and enriched in H2-K1 and lncRNA AW112010, two markers that can distinguish in vivo rare “club-like” progenitors that possess lung epithelial regenerative capacity. These cells were CD74^{high} and Scgb1a1^{low} (see e.g., FIG. 12C). Gene set enrichment analysis revealed that immune responses and antigen presentations were among the most enriched ontologies in these Ccl9^{high} cells based on 1406 differentially expressed genes in this cluster (see e.g., FIG. 12D), similar to H2-K1^{high} progenitors.

Next, pairwise comparisons were performed between the transcriptomes of donor-derived vs endogenous cells for each of the three major airway cell types. There were similar levels of expression of canonical lineage-defining transcripts (e.g. Nkx2-1, Trp63, Scgb1a1, and Foxj1; see e.g., FIG. 4E-4G). Across all lineages in donor-derived cells, there were transcripts encoding MHC genes such as H2-Eb1, B2m and Cd74 among the top 20 differentially expressed genes (see e.g., FIG. 12E). Analyzing each lineage separately, donor-derived basal cells had higher expression of Krt14 and a trend of lower Krt17 (see e.g., FIG. 4E-4F, FIG. 12E-12F); donor-derived secretory cells had significant gene expression differences in proximal-distal patterning markers compared to endogenous cells. For example, donor-derived secretory cells expressed significantly higher levels of distal secretory marker Upk3a and significantly lower levels of proximal secretory markers Reg3g, Cbr2, Chad, and Scgb3a2 compared to endogenous secretory cells (see e.g., FIG. 4E-4F, FIG. 12E-12G). Donor-derived ciliated cells expressed higher levels of Tubb4b, and lower levels of cilia regulatory genes such as Drcl (see e.g., FIG. 4F, FIG. 12E).

### Function of Donor-Derived Cells

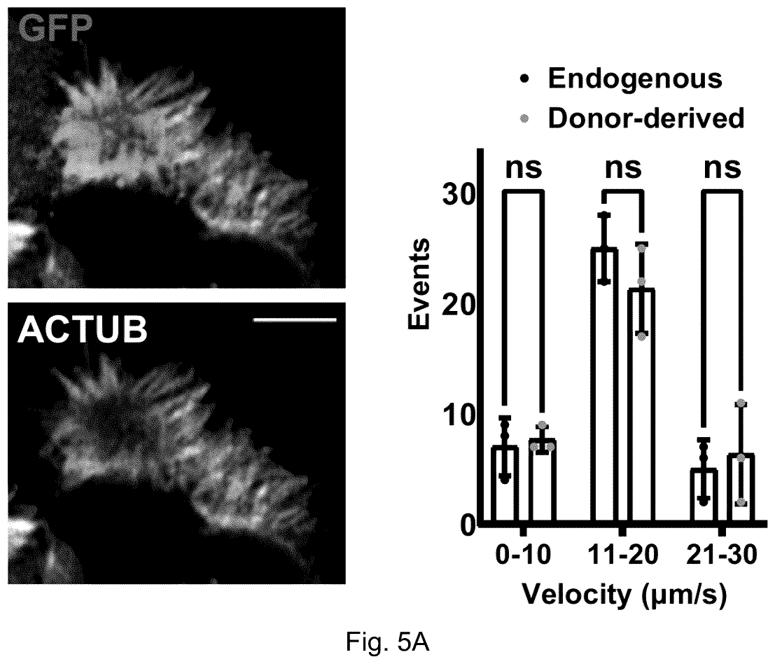

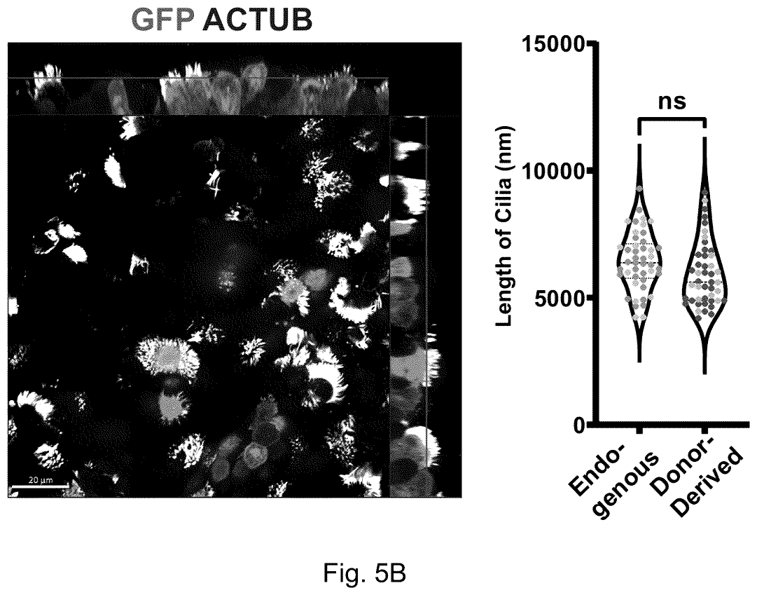

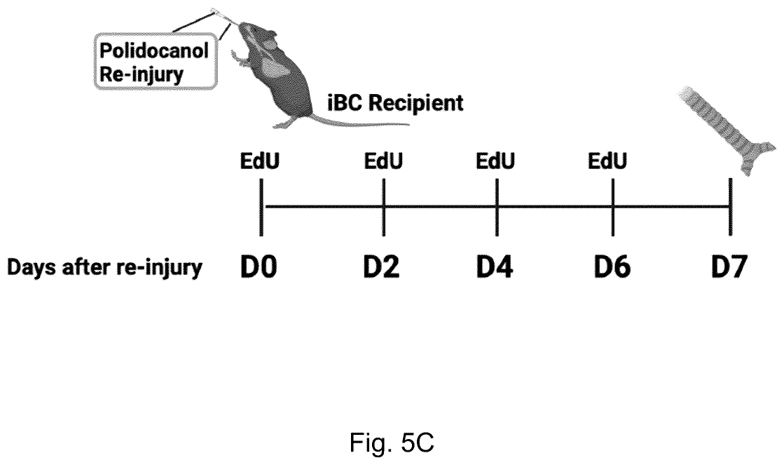

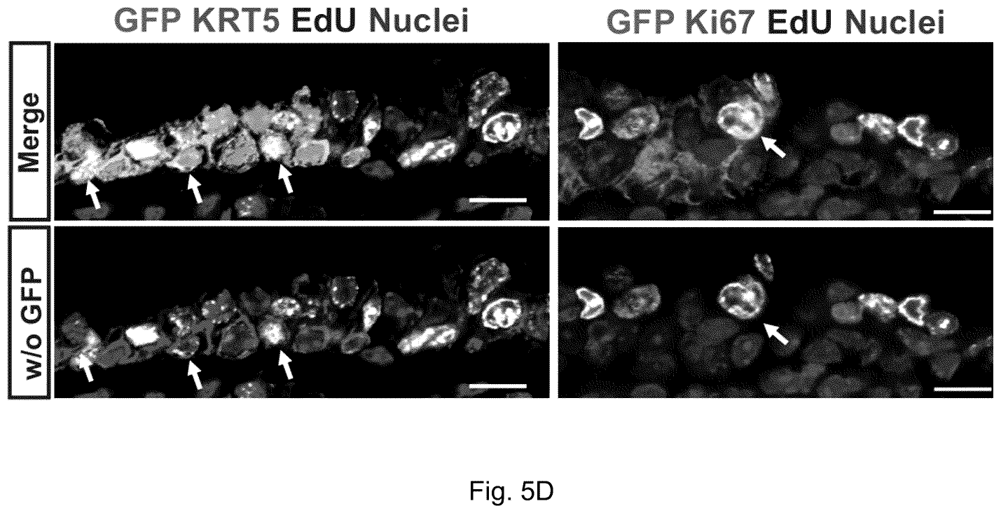

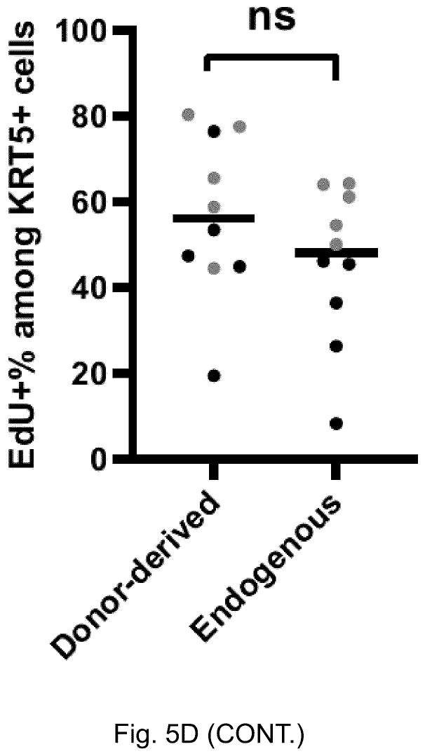

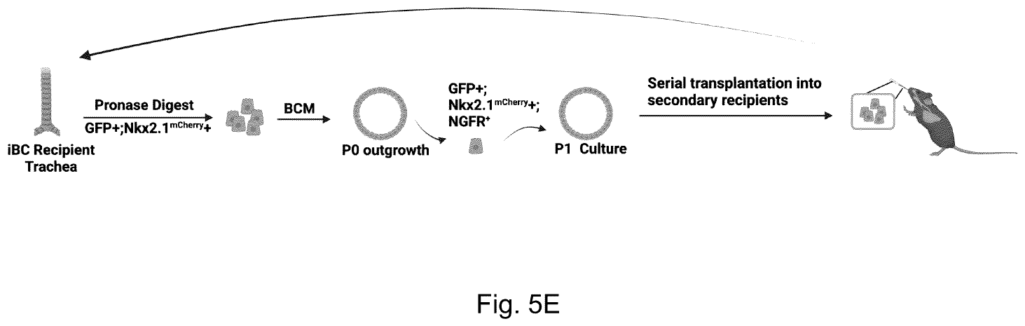

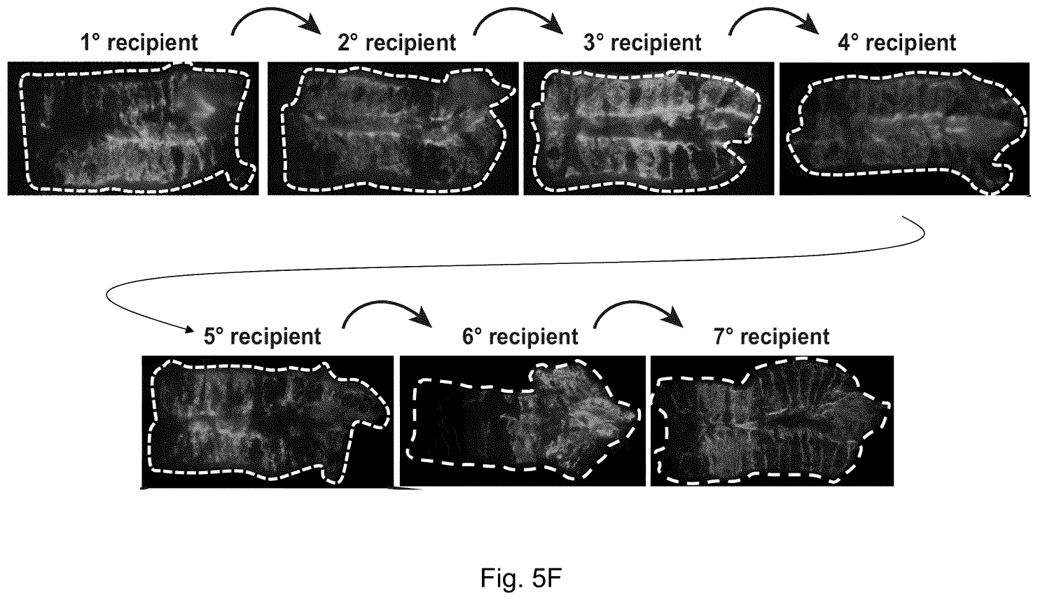

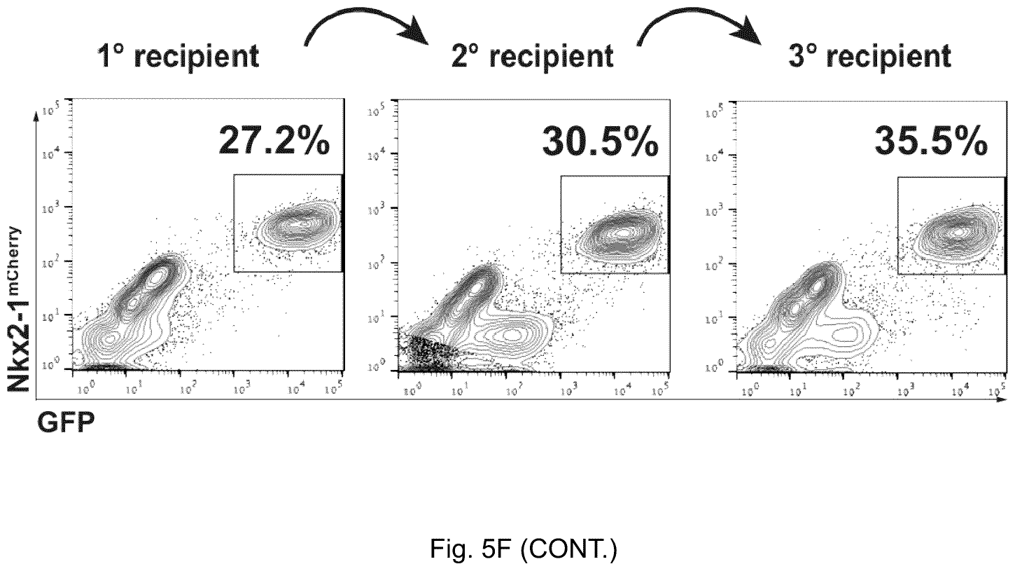

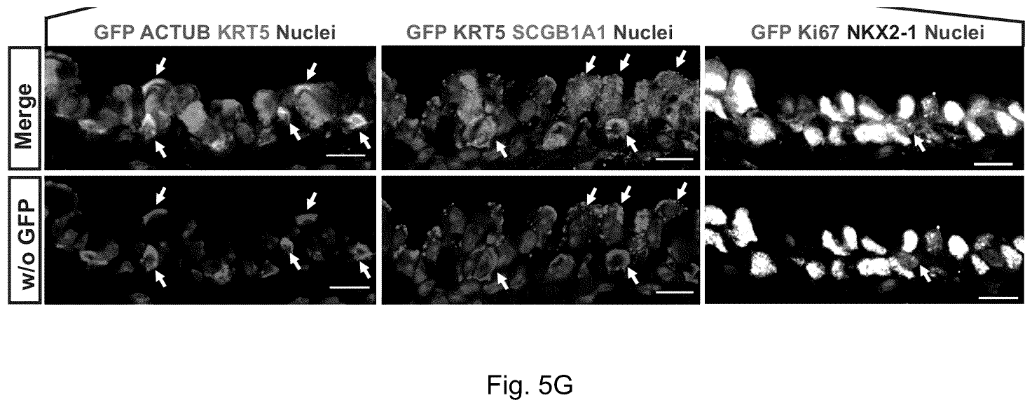

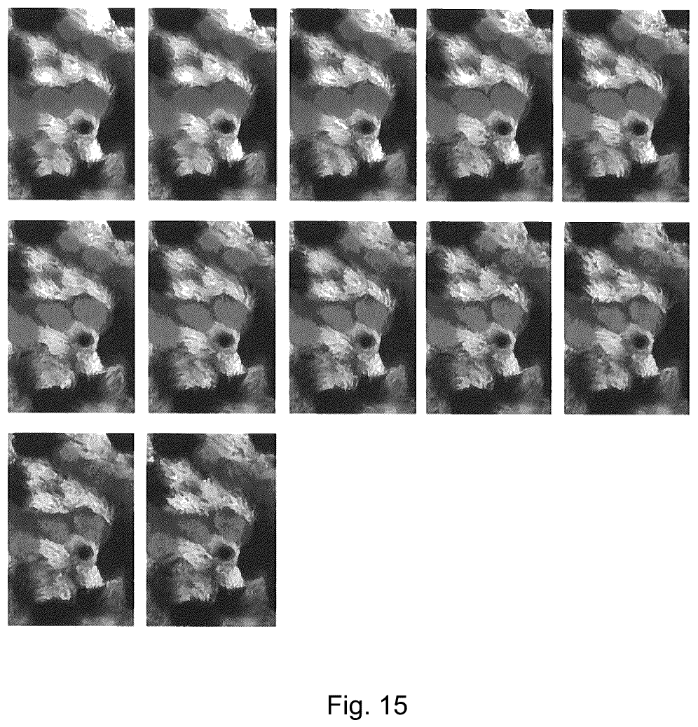

Next, the in vivo function of donor-derived cells was characterized. The morphology and function of motile cilia was quantified in donor-derived vs. endogenous ciliated cells. Using freshly dissected trachea, motile cilia were readily observed by high-resolution microscopy (see e.g., FIG. 15). Immunocytochemistry of motile cilia showed no gross morphological differences between the iBC-derived ciliated cells and endogenous ciliated cells (see e.g., FIG. 5A). Kymograph analysis revealed no statistically significant difference in beating frequencies between donor-derived and endogenous cilia (see e.g., FIG. 5A). Ciliary length from fixed tracheal tissue was also measured, and there was no significant difference in cilia length between donor-derived and endogenous ciliated cells (see e.g., FIG. 5B).

Next, it was tested whether donor-derived basal cells can participate in post-injury repair in response to a second insult in vivo following engraftment. A second round of polidocanol injury was performed in iBC recipients six weeks post-transplantation, and all proliferating cells in vivo were labeled with EdU pulses for 1-week post-injury (see e.g., FIG. 5C). Immunofluorescence microscopy demonstrated GFP+/EdU+/KRT5+ tracheal epithelial cells, indicating engrafted iBCs can re-enter cell cycle and proliferate in response to injury. In addition, GFP+/EdU+/Ki67− tracheal epithelial cells were also observed (see e.g., FIG. 5D), indicating donor-derived cells that had proliferated in the first week after re-injury (during EdU pulse) were able to resume quiescence during the EdU chase period. The percentage of EdU+ donor-derived cells was no different from that of endogenous cells, indicating similar regenerative potency (see e.g., FIG. 5D).

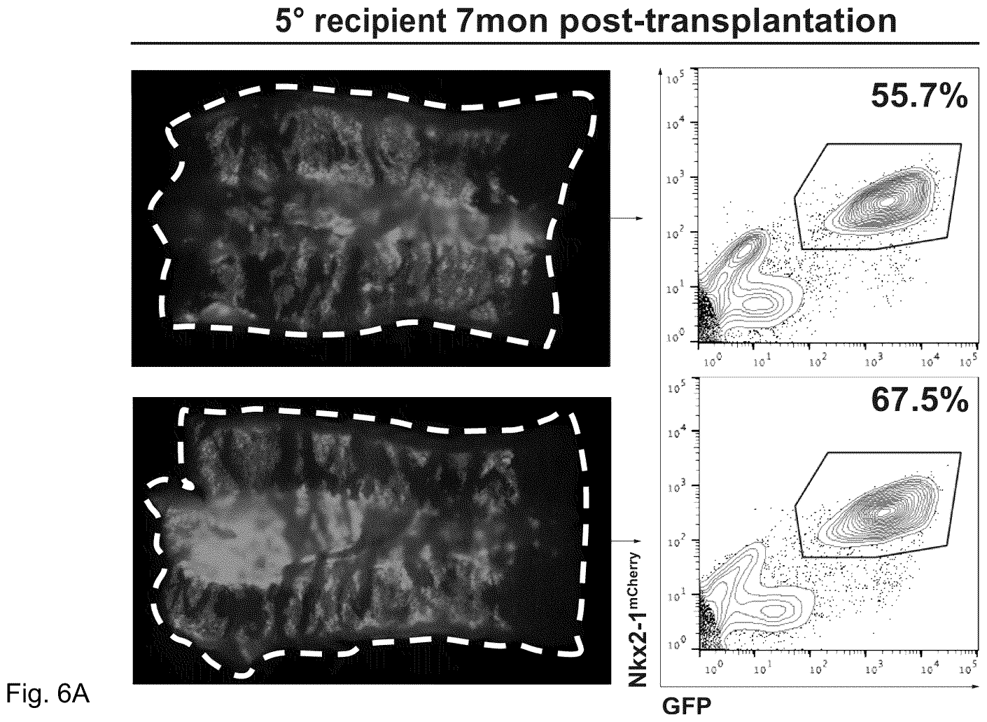

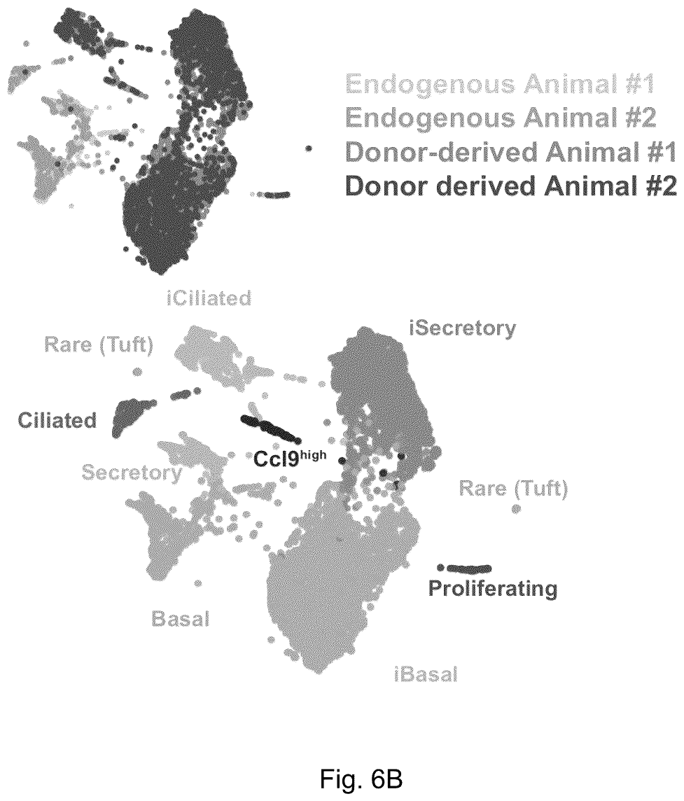

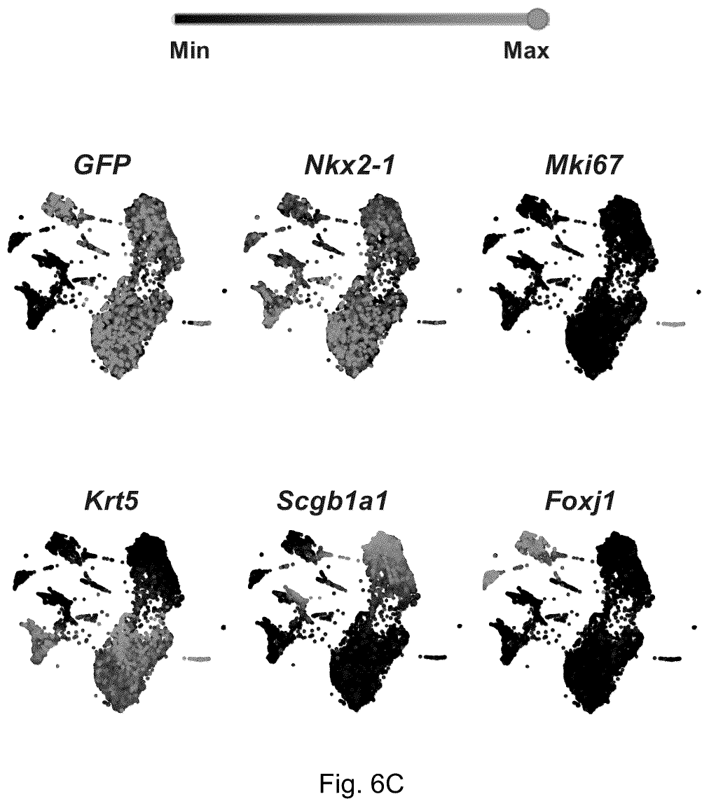

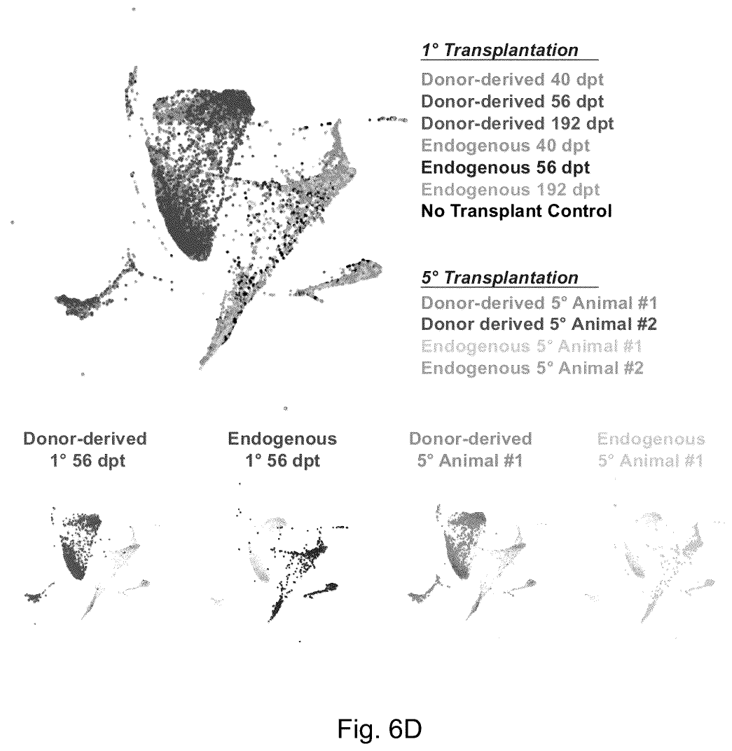

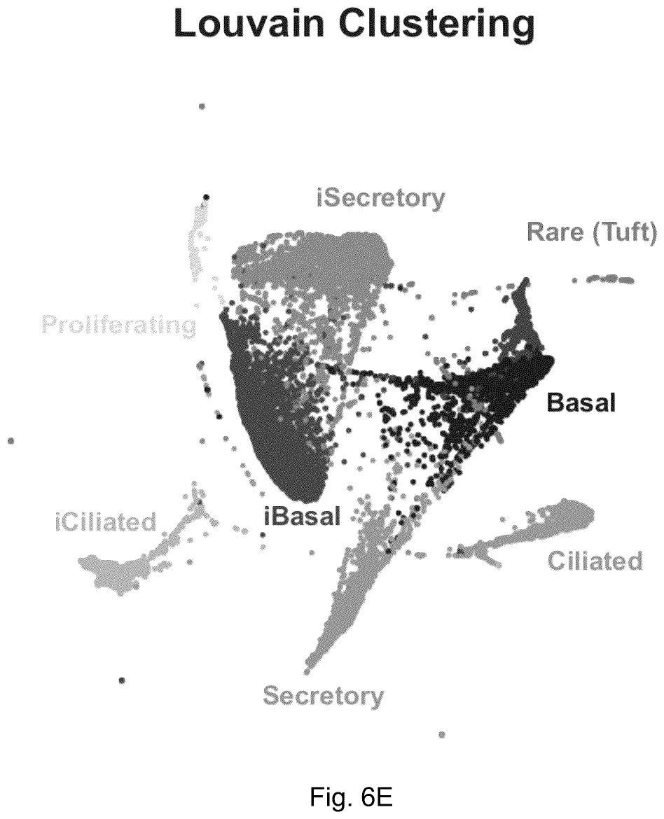

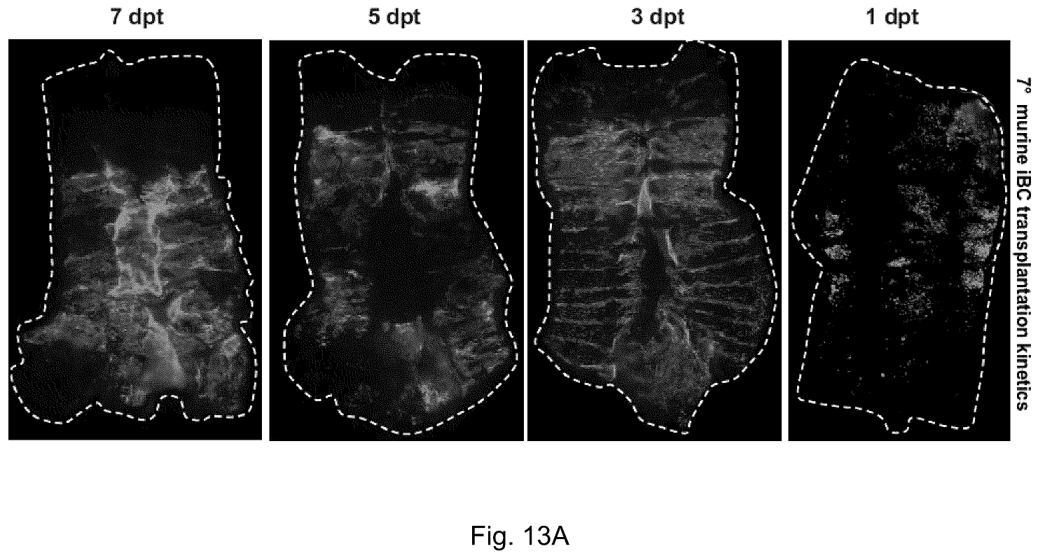

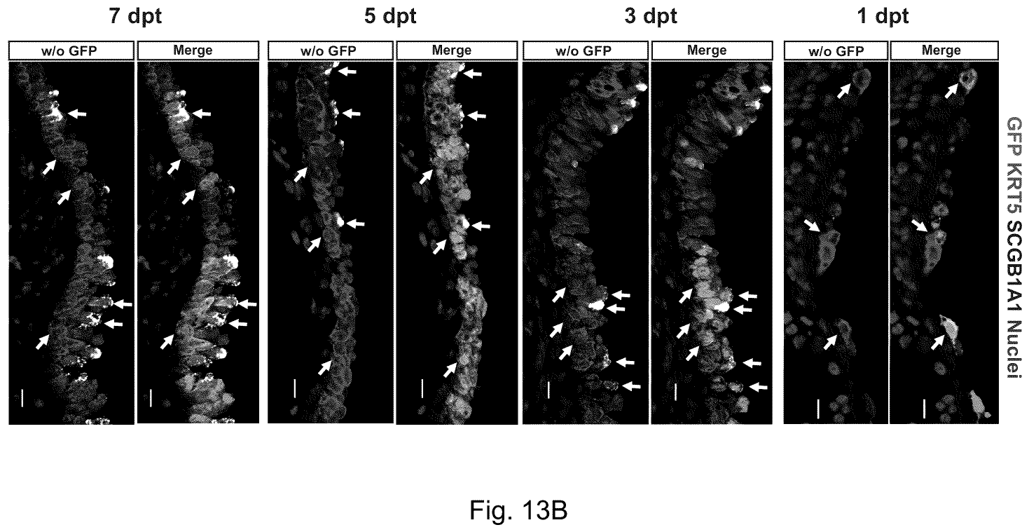

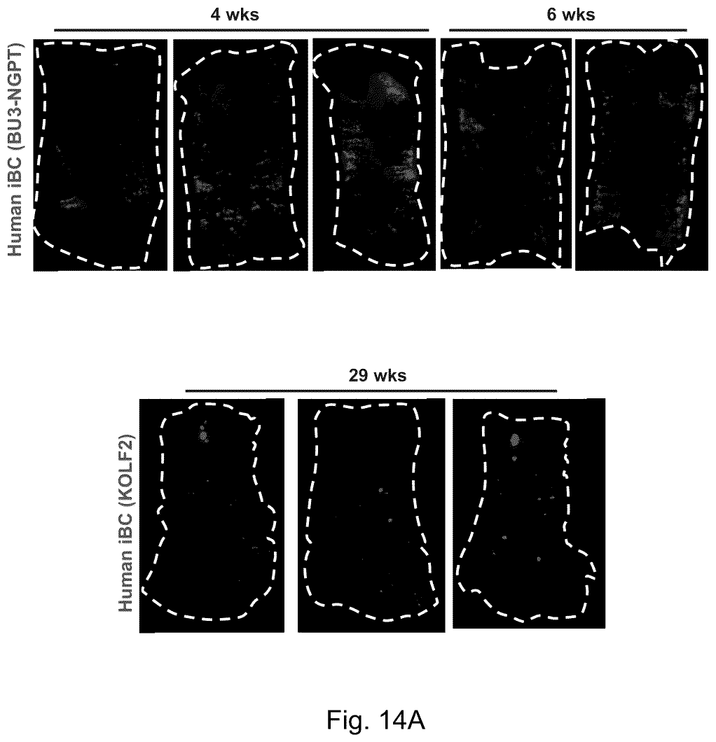

To evaluate whether the key stem cell functions of self-renewal and differentiation had been reconstituted with donor-derived cells, the capacity of engrafted iBCs to contribute to other recipients was next evaluated through serial transplantations. Murine hematopoietic stem cells (HSCs) of the bone marrow have been shown to reconstitute multi-lineage blood lineages after six generations of serial transplantation in myeloablated mice. To demonstrate stem cell self-renewal, GFP+ donor-derived cells were harvested six weeks post-transplantation, they were expanded in BCM to generate the cell number needed for transplantation, and the donor-derived cells were transplanted into secondary polidocanol-injured syngeneic recipients (see e.g., FIG. 5E). Through multiple generations of serial transplantations, robust GFP+ tracheal epithelial repopulating capacity was observed, undiminished even after seven generations of transplantation (see e.g., FIG. 5F-5G). By immunofluorescence, donor-derived cells retained trilineage differentiation potency in vivo (see e.g., FIG. 5H). To investigate whether the transcriptomic profile of the donor-derived cells changed over the generations of secondary transplantations, scRNA-Seq was performed of the tracheal epithelium of two fifth generation recipients (see e.g., FIG. 6A). The donor-derived cells, while contributing to all three airway epithelial lineages in the fifth-generation recipients, still clustered separately from their endogenous counterparts (see e.g., FIG. 6B-6C). Integrating this dataset with that of the first-generation recipients shown in FIG. 4A-4G without harmonization, it was found that all donor-derived cells clustered together and were indistinguishable by Louvain Clustering (see e.g., FIG. 6D-6E), indicating a stably distinct transcriptomic profile of donor-derived cells over time. In addition, to test whether the regenerative potential of iBCs exhaust over time, a time-series experiment was performed, harvesting 7th generation serial transplantation recipients at one day, three days, five days, or seven days post-transplantation. Similar to the findings in primary (first generation) recipients (see e.g., FIG. 11A-11D), the donor-derived cells were again able to generate small colonies and differentiate into secretory cell lineages at 3 dpt, and form pseudostratified epithelium at 5 dpt, which was further established by 7 dpt (see e.g., FIG. 13A-13B), indicating maintenance of regenerative potency. In total more than 20 months elapsed between first transplantation and final harvest of the last transplanted generation of serial recipients, indicating both the longevity of the iBCs as well as their self-renewal capacity.

### Airway Epithelial Reconstitution by Transplantation of Human Primary BC or iBC

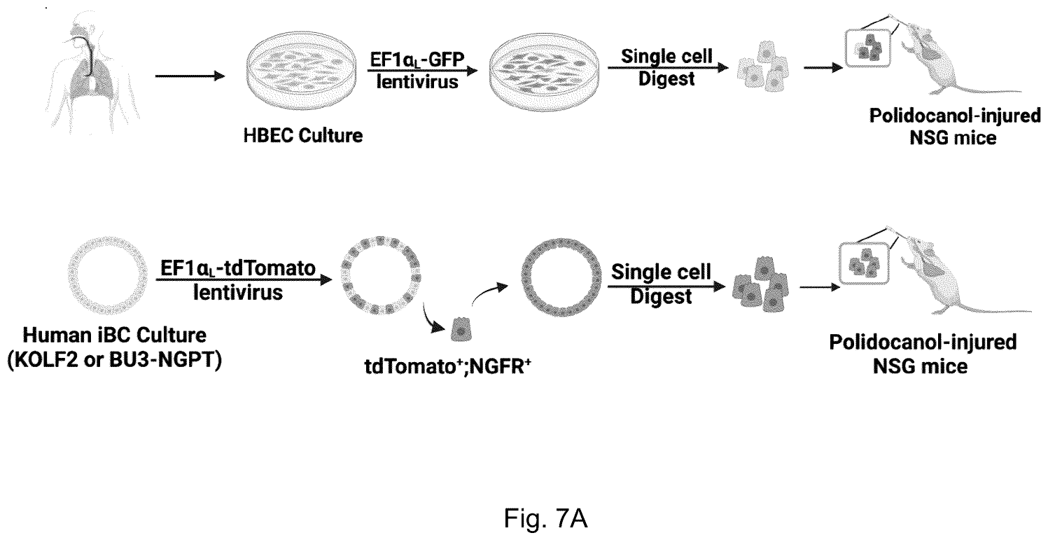

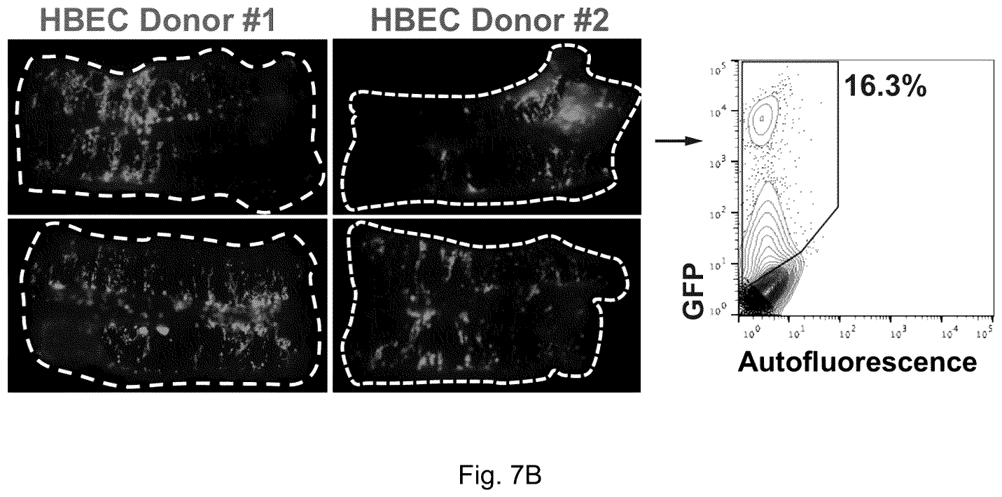

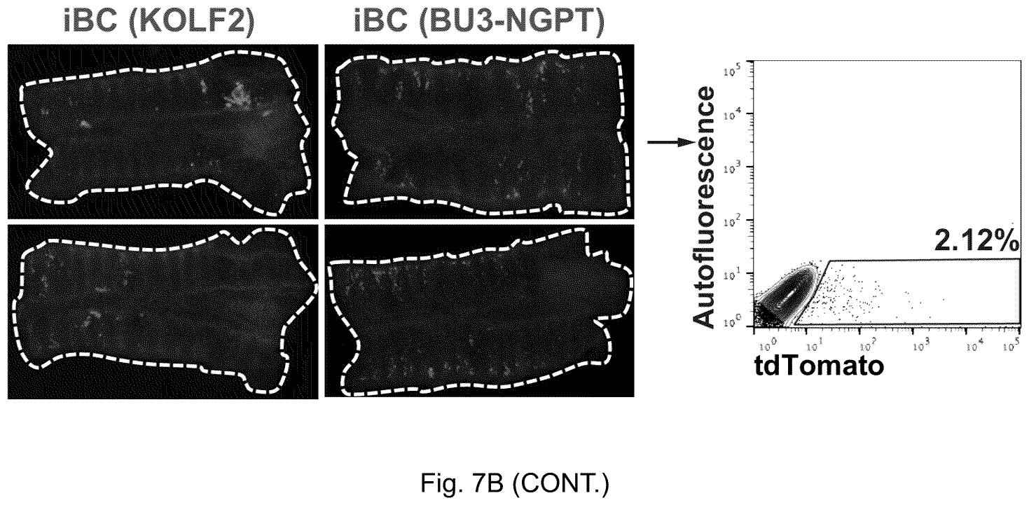

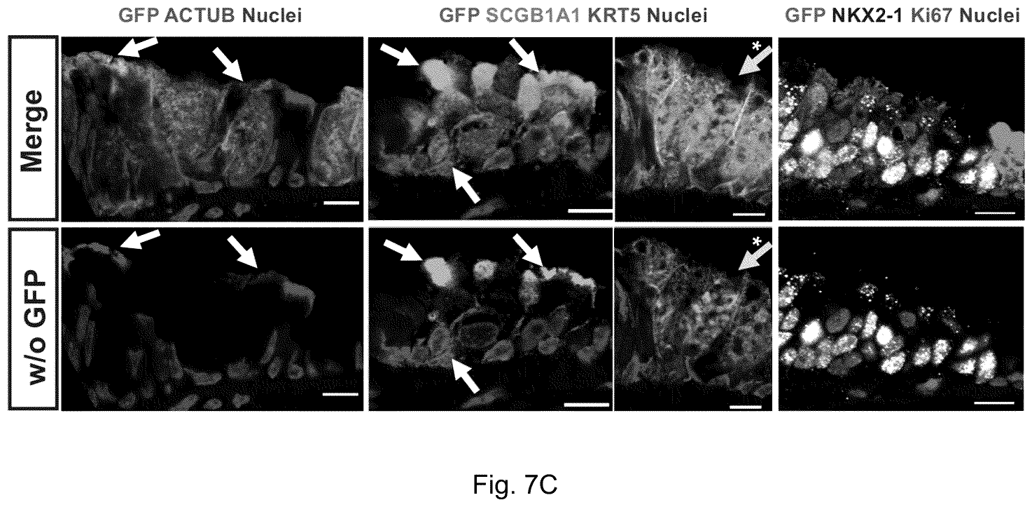

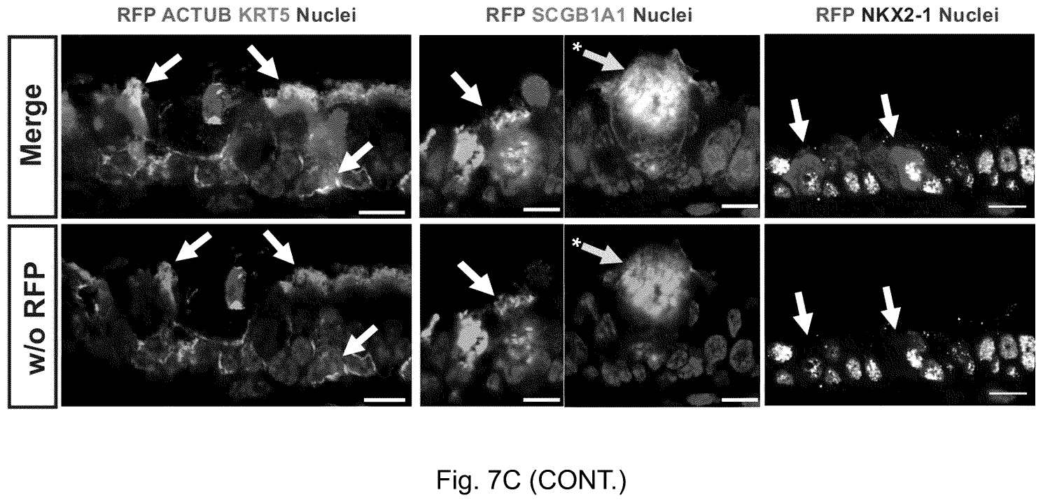

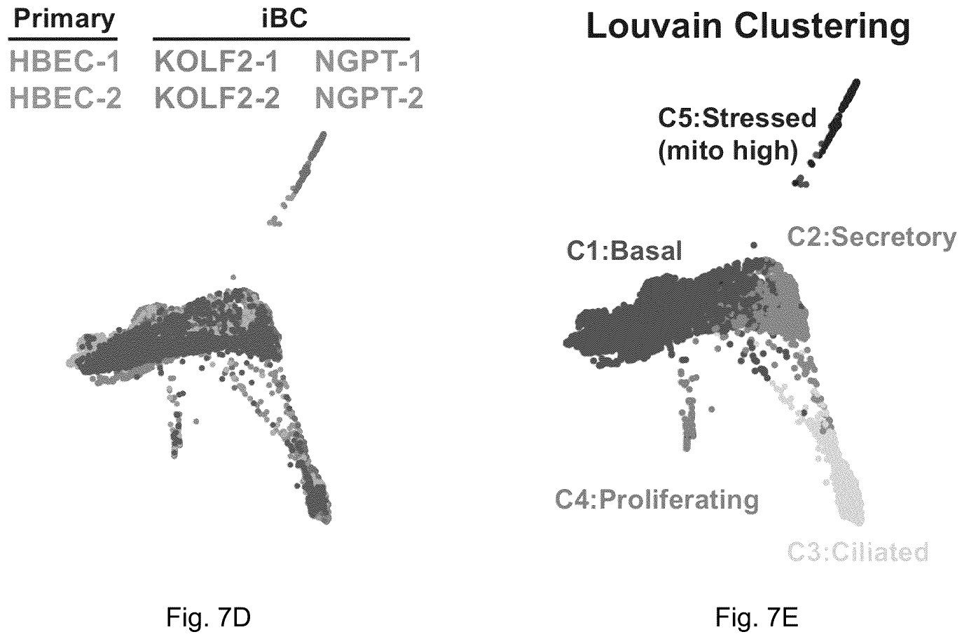

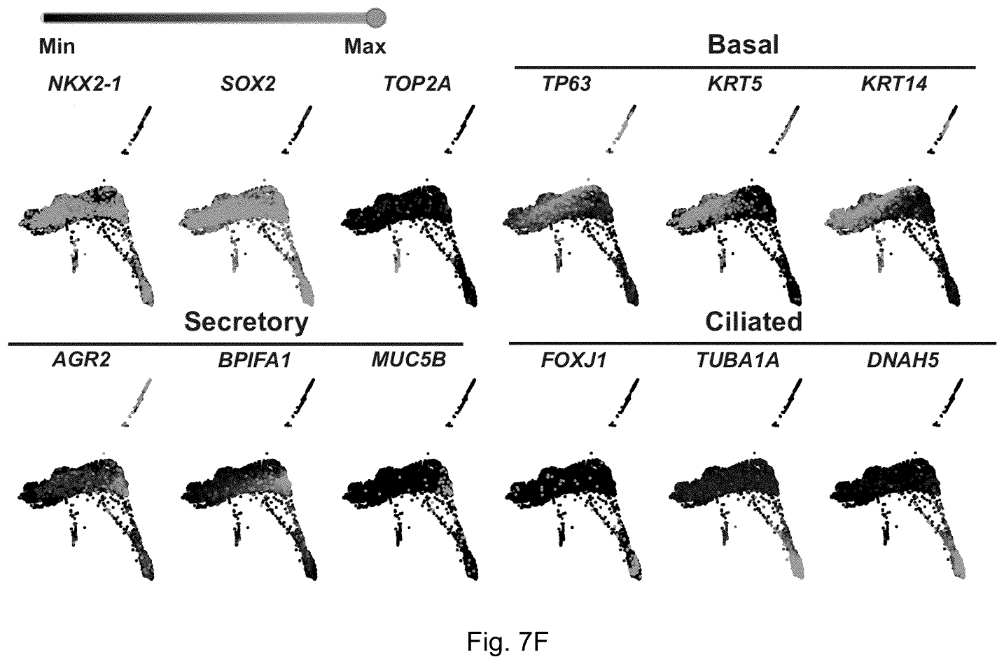

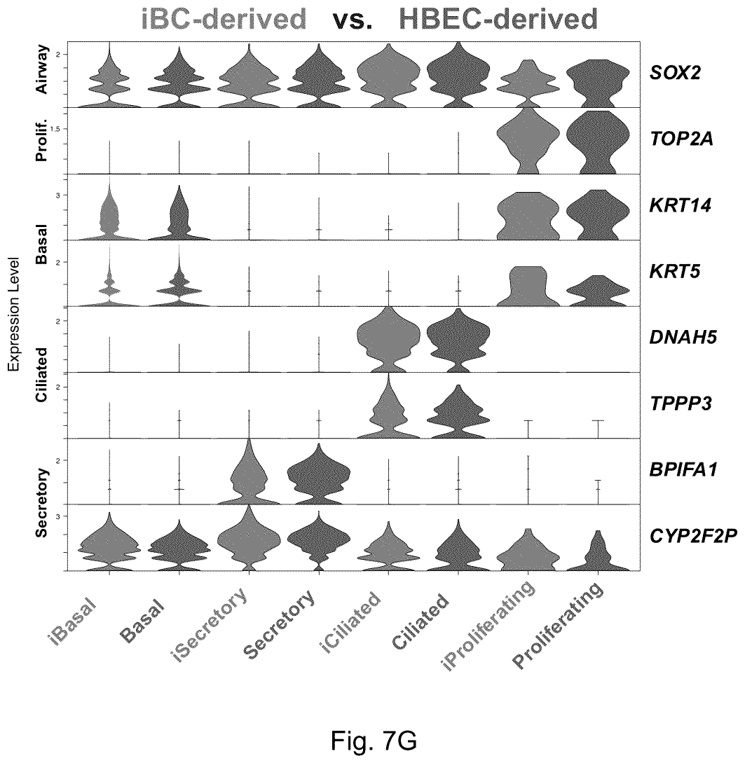

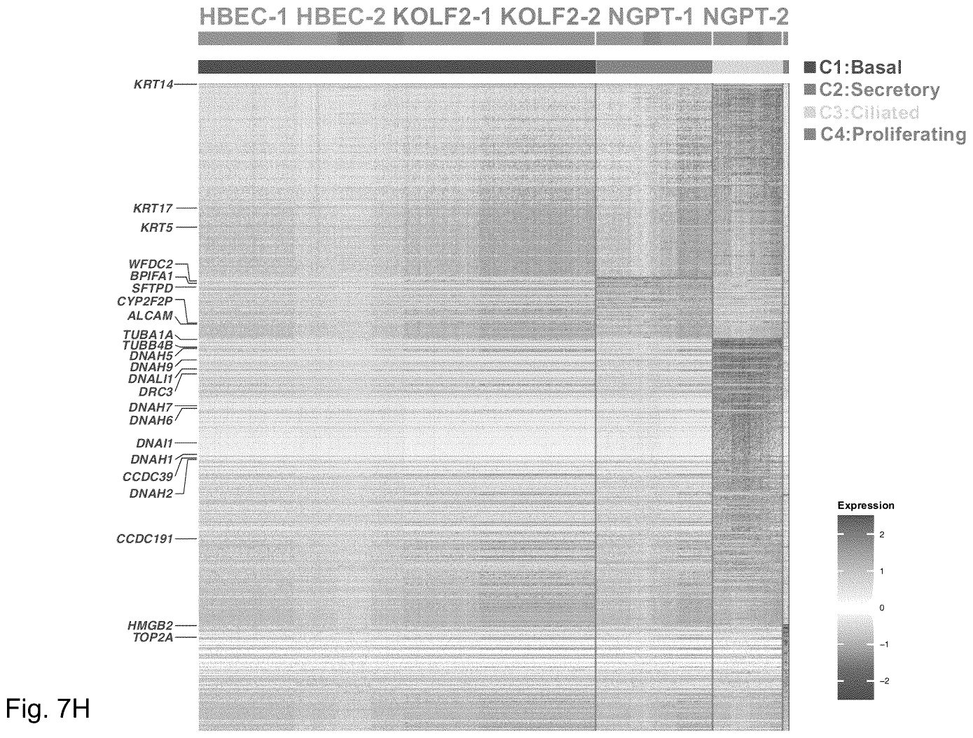

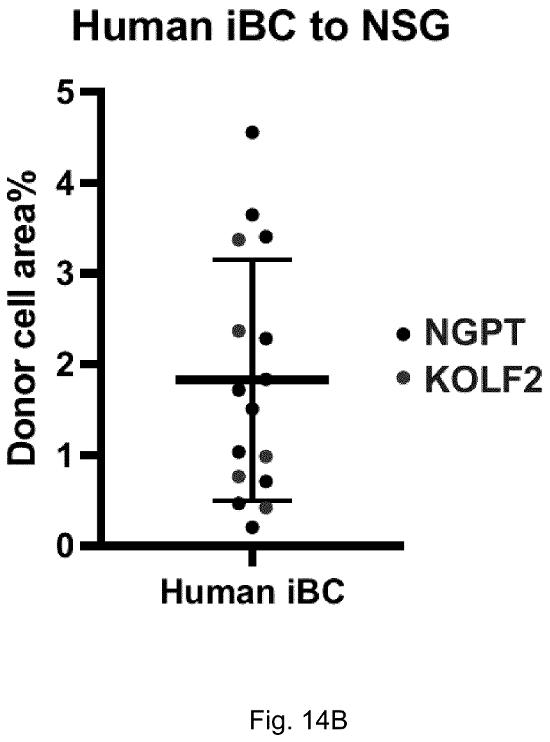

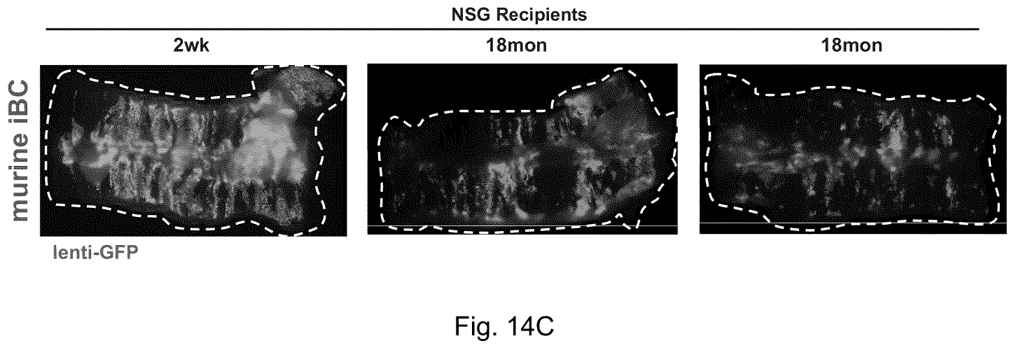

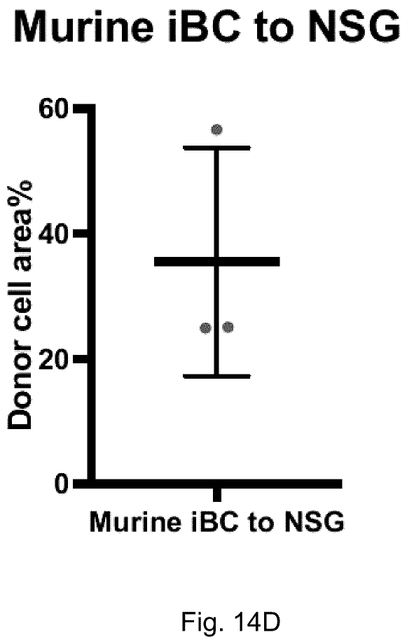

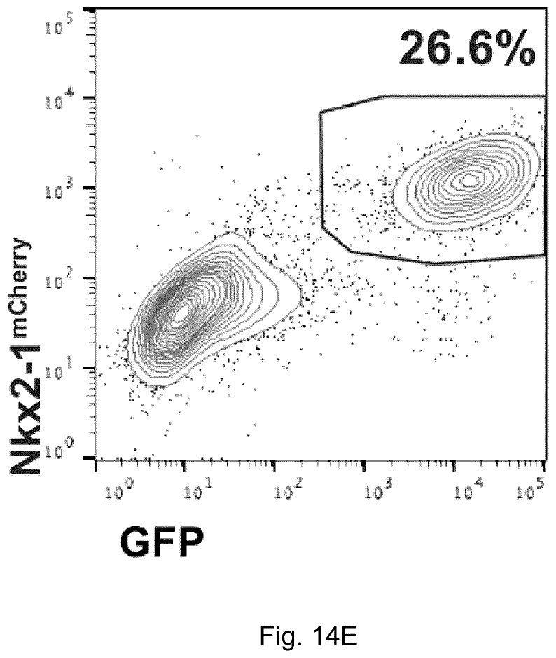

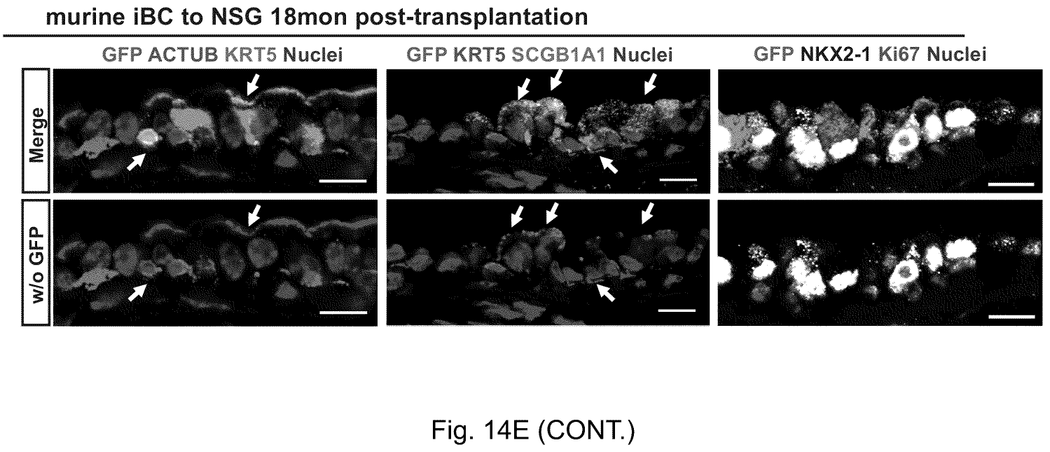

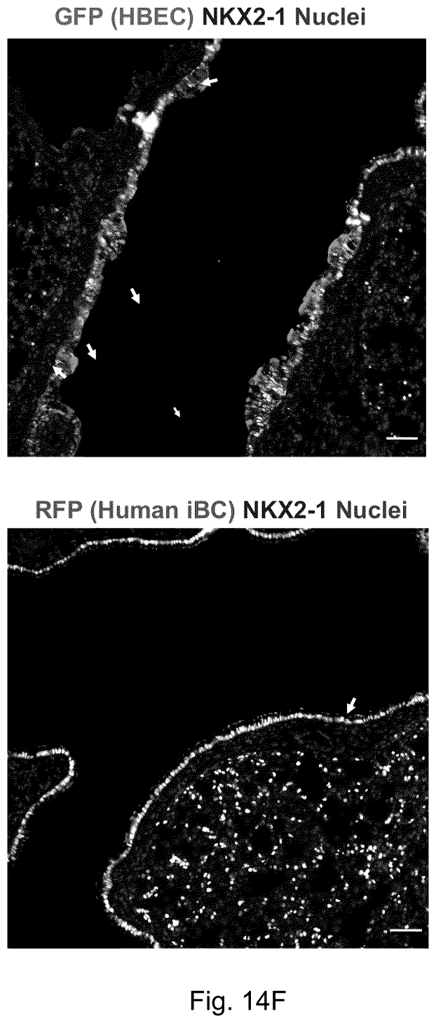

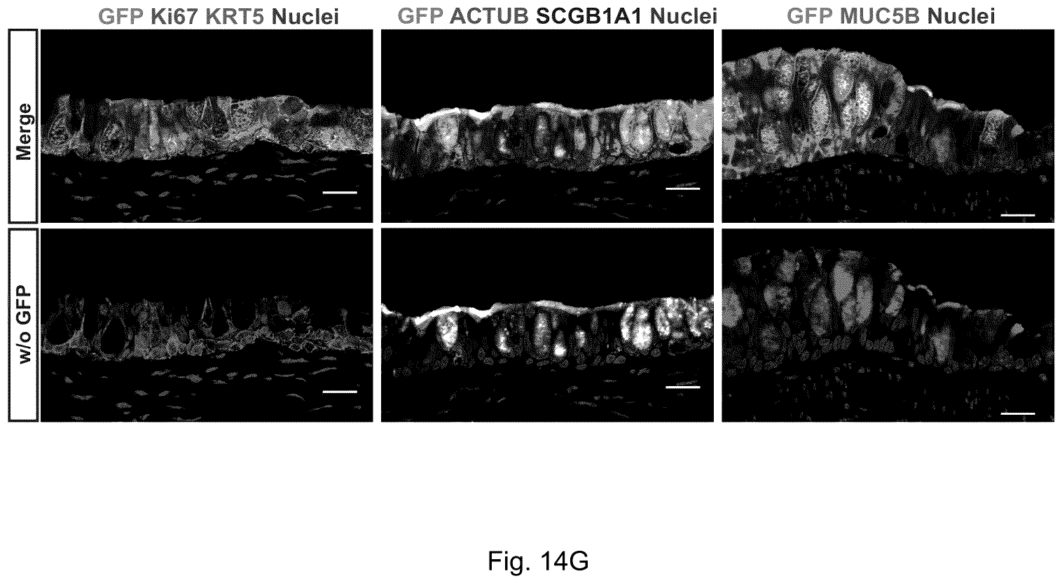

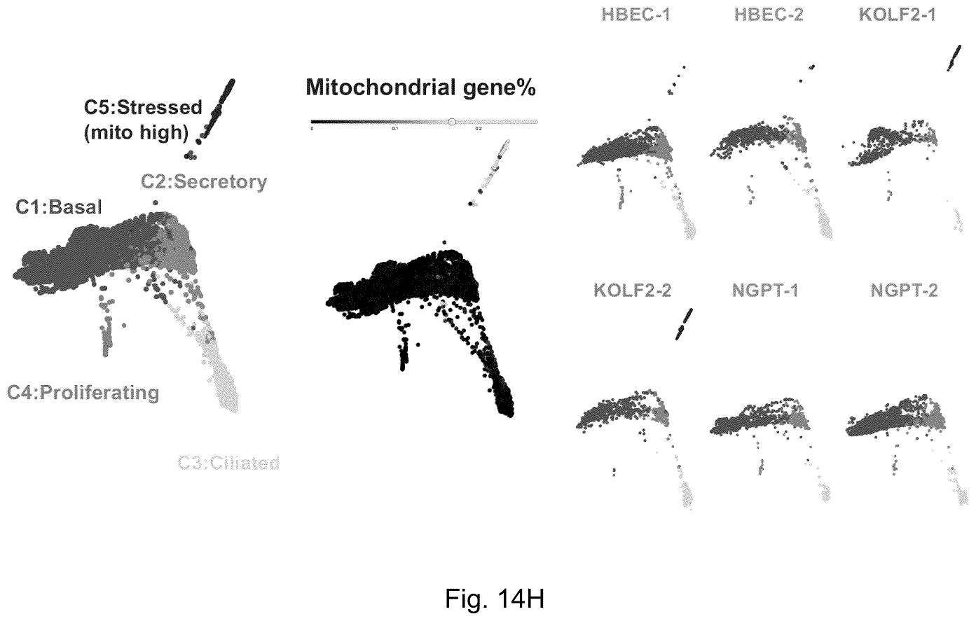

Having established the airway transplantation approach with mouse BCs and iBCs, it was next sought to further the pre-clinical model by transplanting human primary BCs (human bronchial epithelial cells, HBECs) and human iBCs. For HBEC transplantation, cryopreserved P1 HBECs from two different donors were thawed, and the cells were tagged with a GFP-expressing lentivirus before transplantation into NSG recipient mice following polidocanol tracheal injury (see e.g., FIG. 7A). For human iBC transplantation, 3D cultures of two human iBC lines of different genetic backgrounds (KOLF2 and BU3 NGPT) were prepared, and the cells were tagged with a tdTomato-expressing lentiviral vector. tdTomato+/NGFR+iBCs were purified, and these cells were transplanted into NSG recipient animals (see e.g., FIG. 7A). Donor-derived cells from both HBECs and iBCs were detected in recipient tracheas six weeks post-transplantation by whole mount microscopy and flow cytometry (see e.g., FIG. 7B, FIG. 14A). HBEC donors (N=4) contributed to 15.97+/−1.14% of recipients' tracheal epithelium, while human iBCs (KOLF N=5; NGPT N=11) contributed to 1.831+/−1.327% (see e.g., FIG. 14B). Immunofluorescence indicated that human donor-derived cells retained lung fate (NKX2-1+) and contributed to basal (KRT5+), secretory (SCGB1A1+), and ciliated (ACTUB+) lineages in each recipient (see e.g., FIG. 7C). SCGB1A1+ cells of goblet-like morphology were found (see e.g., FIG. 7C, arrows with asterisks), which are normally found in human, but not mouse, tracheas. Because human cell xeno-transplantation into NSG mice resulted in markedly fewer engrafted cells compared to the prior syngeneic mouse cell into immunocompetent mice transplants, it was considered whether this was due to the recipient strain. Murine GFP+iBCs were transplanted into NSG recipients, and GFP+ airway epithelial tri-lineage progeny was observed at 18 months post-transplantation, with similar efficiencies to what had been observed in immunocompetent recipients (see e.g., FIG. 14C-14E).

Given human basal cells extend more distally in the respiratory tree compared to their murine counterparts, the intrapulmonary airways of recipients was next studied. GFP+HBEC− derived ciliated and secretory cells comprising a columnar epithelium that was taller than the adjacent murine host epithelium was observed (see e.g., FIG. 14F-14G). Human donor-derived KRT5+ basal cells and MUC5B+ goblet-like cells, which are not normally present in mouse intra-pulmonary airways (see e.g., FIG. 14G), were detected. Human iBCs, in contrast, contributed mostly to the tracheal epithelium with only rare cells found in the intrapulmonary airways (see e.g., FIG. 14F).

To more extensively characterize the molecular phenotypes of the transplanted human cells and compare transplanted HBECs with human iBCs, scRNA-Seq was performed on GFP+ or tdTomato+ cells isolated from six recipient animal tracheas (two HBEC Donor #2 recipients, two KOLF2 iBC recipients, and two BU3 iBC recipients) six weeks post-transplantation. Human single cell transcriptomes of putative engrafted cells, visualized by SPRING, overlapped across all six recipients without any data harmonization, indicating a high level of similarity regardless of origin (see e.g., FIG. 7D). Louvain clustering revealed five main clusters, annotated based on canonical lineage defining markers as basal (KRT5, TP63), ciliated (FOXJI, DNAH5), secretory (AGR2, MUC5B), proliferating (TOP2A), and a minor stressed population with high mitochondrial gene percentage (see e.g., FIG. 6E-6F, FIG. 14H). All human samples contributed to all major clusters (see e.g., FIG. 7H, 14H). Donor-derived cells from HBECs and iBCs were highly similar to each other in all three lineages based on canonical gene expression and were not distinguishable by Louvain clustering (see e.g., FIG. 7G-7H).

### DISCUSSION

These results demonstrate in vivo airway epithelial engraftment of mouse and human primary BCs or iBCs. Using directed differentiation of PSCs to prepare exogenous airway basal-like cells for transplantation provided an accessible alternative to primary cells, which can be challenging to obtain or expand from patients. Transplantation of basal cells, whether primary or engineered from PSCs, resulted in reconstitution of the endogenous basal stem cell compartment of the pulmonary airways, with engrafted cells capable of long-term self-renewal and durable multipotent differentiation capacity for at least two years in vivo.

Reconstitution of a tissue resident stem cell compartment with primary and engineered cells at high efficiency in an immunocompetent host has considerable implications for regenerative therapy. These findings extend those of prior reports of airway epithelial transplantation, where immunodeficient recipient animals displaying evidence of grafted epithelial cells were followed for shorter periods with less formal tests of in vivo stem cell functional reconstitution; see e.g., Vaughan et al. (2015) Nature 517, 621-625; Nichane et al. (2017) Nat Methods 14, 1205-1212; Miller et al. (2018) Stem Cell Rep 10, 101-119; Kathiriya et al. (2020) Cell Stem Cell 26, 346-358.e4; Kathiriya et al. (2022) Nat Cell Biol 24, 10-23; Liao et al. (2022). Iscience 25, 104262; Louie et al. (2022) Cell Reports 39, 110662; Ghosh et al. (2016) Am J Resp Cell Mol 56, 1-10.

Mouse primary BCs and iBCs, when transplanted into syngeneic recipients without immunosuppression, can replace more than 50% of the endogenous tracheal epithelium after a single treatment. This degree of engraftment efficiency, if performed with gene-corrected cells, can provide sufficient functional replacement of endogenous cells compromised by disease-driving gene mutations. Engraftment of immunocompetent recipients with syngeneic cells also allows for autologous cell transplantation approaches that can be adapted to reconstitute the airways of individuals with cystic fibrosis or primary ciliary dyskinesia using their own iPSC-derived basal cells after in vitro gene editing.

One major hurdle limiting cell transplantation is producing large numbers of donor cells likely to be needed for the procedure. Airway basal cells are the resident stem cells of the mouse trachea and human pseudostratified airway epithelium, but obtaining these cells in sufficient quantity from patients already compromised by lung disease can be challenging. Primary human bronchial cells harvested via bronchoscopy from some patients or from explant lung tissues from others offers one source of cultured basal cells for transplantation, as used herein; however, some hurdles that can limit access to this resource for some patients include: 1) the invasive procedures required to harvest these cells; 2) loss of basal cell phenotype over serially passaged cultures; and 3) retained epigenetic signatures of disease in primary cells. These findings indicate iBCs as an alternative source of cells for transplantation that are easily accessible, archivable, readily gene-edited to correct disease-causing mutations, and have been reprogrammed to erase epigenetic disease-associated signatures. The human transplantation model offers a platform that can be employed to test and compare the in vivo functions, reconstituting advantages and disadvantages of both primary and engineered human basal-like cells. The similarity of the single cell transcriptomes of the progeny deriving in vivo from each human cell type after transplantation indicates either cell type can be used for airway engraftment, at least in the case of normal BCs and iBCs, and studies can focus on how cells from individuals with lung disease function in vivo after engraftment. In addition, long-term studies can focus on the safety of the transplanted cells, screening for potential malignant transformation. In these studies, tumor formation from donor-derived cells was not observed in any recipient animal, including in NSG recipients which are more prone to tumor formation.

One issue to address in transplantation of exogenously cultured cells is their similarity or difference from their endogenous in vivo counterparts, particularly for PSC-derived lineages that can be less mature than primary lineages in some cases. The transplanted mouse primary BCs contributed to major (basal, secretory, ciliated) and rare (ionocyte, neuroendocrine, tuft, hillock) airway epithelial lineages that were almost indistinguishable from their endogenous counterparts according to transcriptomic analyses. In contrast, iBC-derived cells, while contributing to all major airway epithelial lineages with similar expression of most canonical marker transcripts, remained distinct from their corresponding endogenous lineages in terms of their global transcriptional profiles. Both iBC-derived basal and secretory lineages exhibited differences in regional patterning. For example, iBC-derived secretory cells exhibited equal Scgb1a1 transcript expression to endogenous tracheal secretory cells, but lower Reg3g, Muc5b, Scgb3a2, Chad, and Cbr2, indicating a less proximal (tracheal) molecular phenotype based on markers of club cell proximal-distal patterning. Some of these differentially expressed gene have can also be markers of a proximally localized club cell subpopulation. One explanation for such differences is that iBCs can be capable of giving rise to epithelial lineages representing molecular phenotypes of both proximal and distal airways despite their engrafted location in the trachea. Studies can test this hypothesis, including comparative profiling with intrapulmonary endogenous lineages. Studies can also explore the donor-derived intra-pulmonary basal cells observed in some recipients of mouse iBCs or HBECs. Since intra-pulmonary basal cells in mouse airways are infrequently observed, except in settings of injury, studies can determine the mechanisms and stem cell niches responsible for these persistent intra-pulmonary airway phenotypes. The derivation from human donor-cells of intra-pulmonary tall columnar goblet cells, a phenotype also not normally found in the murine airways, can also be studies, as this finding indicates this fate is determined by the cell of origin in these studies.

Despite their transcriptomic differences, mouse iBC-derived lineages exhibited normal in vivo function, such as motile cilia beating from multiciliated cells and post-injury proliferation from basal cells. In contrast to mouse iBC derivatives, human primary, and iBC-derived lineages across all three different genetic backgrounds showed high levels of transcriptomic similarity in vivo after engraftment.

In summary, these results establish airway stem cell transplantation with exogenous mouse and human primary or PSC-derived basal stem cells to durably reconstitute the airway stem cell compartment in vivo. This study thus allows for cell-based therapies, whereby autologous gene-corrected cells can be employed for in vivo reconstitution of airway epithelial function for individuals with genetic airway diseases.

This work establishes the feasibility of transplantation of primary and PSC-derived cells into mouse trachea. Studies can be performed related to clinically relevant approaches, including with regard to epithelial conditioning regimens. The transplantation model includes pre-exposure to polidocanol to injure the murine tracheal epithelium. Clinically relevant conditioning regimens include localized airway epithelial denudation with detergents and/or ex vivo perfusion systems to maintain organ function in recipients of airway instilled cells that can facilitate clinical adaptation of the approach (see e.g., Dorrello et al. (2017). Sci Adv 3, e1700521; the contents of which are incorporated herein by reference in their entirety). In addition, localized delivery technologies such as bronchoscopy can be used to selectively graft locally prepared lobes or intrapulmonary airways.

### Experimental Model and Study Participant Details

### Animals:

All studies involving mice were approved by an Institutional Animal Care and Use Committee. All transplantation experiments were performed on 8-16 week old adult male mice, as follows. For primary BC transplantations, donor cells were harvested from 8-16 weeks old male C57BL/6J background UBC-GFP transgenic mice animals (JAX004353). Recipient WT immunocompetent C57BL/6J mice were obtained from JACKSON LABORATORY (JAX000664). For murine iBC transplantations, WT immunocompetent recipient mice were F1 offspring generated by crossing 12951/SvlmJ Males (JAX002448) and 129X1/SvJ Females (JAX000691). For human primary and iBC transplantation experiments, 10-12 week old NSG recipient mice (JAX005557) were used.

### Cell Lines:

Primary murine basal cell culture was established with harvested airway basal cells from C57BL/6J animals. Mouse iBCs were generated from a Nkx2.1^{mCherry} reporter R1 ESC line (129X1SvJx 129S1/Sv background).

Primary human bronchial epithelial cells (HBEC) were generated from lung tissue of deceased donors. Human iBCs were generated from KOLF2 and BU3 NGPT human iPSCs.

The culture conditions for different cell lines are detailed in the “methods details” section below.

### Methods Details

Generation and Maintenance of Murine iBCs

Cell differentiation was performed in a complete serum free differentiation medium (cSFDM) comprising a base medium of IMDM (THERMOFISHER, 12440053) and Ham's F12 (CELLGRO, 10-080-CV) with B27 Supplement with retinoic acid (INVITROGEN, 17504-44), N2 Supplement (INVITROGEN, 17502-048), 0.1% bovine serum albumin Fraction V (LIFE TECHNOLOGIES, 15260-037), monothioglycerol (SIGMA, M6145), GLUTAMAX (LIFE TECHNOLOGIES, 35050-061), ascorbic acid (SIGMA, A4544), and PRIMOCIN (INVIVOGEN, ANTPM1). One million mouse embryonic stem cells (ESCs) carrying a Nkx2-1^{mCherry} reporter were first cultured in 10 mL cSFDM in suspension for 60 hr to allow embryonic body (EB) formation. Directed differentiation was performed by adapting an approach for deriving airway organoids from mouse PSC. Briefly, to induce definitive endoderm fate, EBs were dissociated into a single cell suspension and cultured in cSFDM supplemented with 50 ng/mL recombinant human/mouse/rat Activin A (R&D systems, 338-AC) for 60 hr allowing EBs to reform in suspension culture. Next, 100 ng/mL recombinant mouse Noggin (R&D systems, 1967-NG) and 10μM SB431542 (SIGMA, S4317) were applied for 24 hr to generate anterior foregut endoderm. Then, EBs were dissociated, and single cell passaged onto a 6-well tissue culture plate coated with 200 μL of growth factor reduced 3D-MATRIGEL (CORNING, 354230). To induce NKX2-1 expression, adherent cells were cultured in cSFDM supplemented with 100 ng/mL recombinant mouse Wnt3a (R&D systems, 1324-WN-010) and 10 ng/mL recombinant human BMP4 (R&D systems, 314-BP) for 7-8 days depending on cell morphology and growth kinetics (typically 8 days; until total differentiation day D14). During the first 48 hr (D6-8) the Wnt3a/BMP4 supplemented specification medium was further supplemented with 100 nM Retinoic acid (SIGMA, R2625) and 10μM Y-27632 (TOCRIS, 1254). On D13-14 the cells were incubated at 37° C. for 1 hr in 1 mg/ml each of Collagenase IV (THERMOFISHER, 17104019) and Dispase (GIBCO, 17105-041) to digest the MATRIGEL bed, and the released epithelial spheres were isolated and washed with PBS through two slow centrifugations at 100×g. The resulting cell pellet was dissociated with 1 mL warm 0.05% trypsin-EDTA (GIBCO, 25300062) for 8 min at 37° C., followed by inactivation with 1 mL of cold fetal bovine serum (GIBCO, 16141079). Cells were sorted by flow cytometry on the basis of EPCAM (BV421 anti-CD326, BD BIOSCIENCES, 563214) and Nkx2-1^{mCherry}. EPCAM+/Nkx2-1^{mCherry}+ cells were then resuspended in 3D-MATRIGEL at a density of 500 cells per μL and pipetted in 15-20 μL droplets onto the base of tissue culture plates.

After 20 min at 37° C. to allow gelling of the MATRIGEL, airway medium composed of cSFDM containing 250 ng/mL recombinant human FGF2 (R&D systems, 233-FB), 100 ng/mL recombinant human FGF10 (345-FG), 10 μM Y27632, 100 ng/mL Heparin (SIGMA, H3393) to induce airway lineage. After 10-14 days, airway organoids were again dissociated into single cell suspension. The 3D-cultures were first incubated at 37° C. in 1 mg/mL dispase and 1 mg/mL collagenase, then collected and centrifuged at 300×g for 5 min. The cell pellet was then dissociated with 1 mL warm 0.05% trypsin-EDTA for 8 min at 37° C., and then inactivated with 1 mL of cold fetal bovine serum. The cells were then strained through a 40 m cell filter and centrifuged at 300×g for 5 min. Resulting cells were purified by flow cytometry sorting Nkx2-1^{mCherry+} cells and plated in 3D Matrigel for culturing in Basal Cell Medium (BCM) comprising: a base medium of cSFDM supplemented with 250 ng/mL recombinant human FGF2 (R&D systems, 233-FB), 100 ng/mL recombinant human FGF10 (R&D systems, 345-FG), 10 μM Y27632 (TOCRIS, 1254), 100 ng/mL Heparin (SIGMA, H3393), 1 μM A83-01 (TOCRIS, 2939) and 1 μM DMH-1 (TOCRIS, 4126). After 10-14 days, epithelial spheres outgrowths were single cell digested as before, and “iBCs” were purified by flow cytometry sorting Nkx2-1^{mCherry} and NGFR (ABCAM, ab8875 or ab245134) double positive cells as indicated. Nkx2-1^{mCherry}+/NGFR+ cells were then re-plated in 3D MATRIGEL in BCM and serially passaged every 10-14 days without further sorting. Where indicated, these cultures were either prepared for transplantation, characterization, or cryopreservation. For cryopreservation, cells were dissociated into single cells as described above and subsequently frozen in cryopreservation media (90% BCM+10% DMSO) at 500,000 cells/mL at −150° C. To thaw cryopreserved frozen iBC archives, the cryovial was warmed to 37° C. in a water bath and dilute the cell suspension with 9 mL of BCM. Cell suspensions were centrifuged at 300×g for 5 min, before resuspending in MATRIGEL at 500 cells per μL in 3D-MATRIGEL. After 10-15 min of incubation at 37° C., BCM was added and iBC culture was maintained as described above.

### Murine Primary BC Culture

Mouse tracheas (between larynx and carina) were harvested from adult mice (8-16 weeks). The lymphatic, muscular and other surrounding tissues were removed by surgical dissection, and the remaining tracheas were opened longitudinally. Tracheal epithelium was then dissociated by enzymatic digestion using 1.5 mg/mL pronase (ROCHE, 10165921001) in Ham's F12 at 4° C. for 18 hr to create a single cell suspension. To purify live tracheal epithelial cells, collected cells were stained with BV421-conjugated rat anti-mouse EPCAM monoclonal antibody (BV421 anti-CD326, BD BIOSCIENCES, 563214) and the cell viability dye DRAQ7 (ABCAM, ab109202). DRAQ7−/EPCAM^{hi} cells were sorted and resuspended in 3D-MATRIGEL at a density of 500 cells/μL and pipetted as 15-20 μL droplets added onto the base of 12 well tissue culture plates. After 20 minutes of incubation at 37° C. to allow gelling of the MATRIGEL, BCM was added with medium changes every two days of culture. Cells were passaged every 10-14 days with the same method used to passage murine iBC.

### Establishing GFP or GFP-Luciferase Tagged Murine iBC Lines Via Lentiviral Transduction

Established iBCs (after the first NGFR sort) were used for lentiviral transduction as follows. Single cell suspensions were prepared as detailed above. Then, 100 k cells were incubated in suspension with either pHAGE-EF1α_{L}-GFP-W lentivirus (ADDGENE #126686), or CMV-Luciferase-EF1α-copGFP lentivirus (SYSTEM BIOSCIENCES, BLIV511PA-1) at a multiplicity of infection (MOI) of 50 in 200 μL of BCM supplemented with 5 μg/mL polybrene. Cells were incubated with lentiviral particles for 4 hrs total, with the suspension gently agitated every 20 min. Then, cells were diluted with 15 mL of BCM, centrifuged at 300×g for 5 min, and then washed with BCM again and pelleted for resuspension in 3D cultures as detailed above. The initial outgrowth after transduction was purified by flow sorting Nkx2-1^{mCherry}+/NGFR+/GFP+ triple positive cells to establish each GFP or GFP-Luc tagged iBC line.

### HBEC Culture

HBECs (DD008P, DD024O) were obtained from a Center Tissue Procurement and Cell Culture Core. Human lung tissue was procured under an Office of Research Ethics Biomedical Institutional Review Board. Primary HBECs were cultured. Briefly, cryopreserved P1 HBECs were thawed and cultured on PURECOL (ADVANCED BIOMATRIX, 5005)-coated tissue culture dish in BEGM media at two million cells in one 10 cm plate. Two days after establishment of the culture, for GFP tagging, HBECs were transduced with EF1α_{L}-GFP-W lentivirus at MOI=10. Donor HBECs were cultured for a further 5-6 days in BEGM without passaging until the culture reached ˜90% confluence, at which point HBECs were harvested for transplantation without further sorting.

### Human iBC Culture

Human iBC lines (generated from KOLF2 and BU3 NGPT iPSC lines) were derived by directed differentiation and cryopreserved or maintained as stable self-renewing cultures in serum-free, feeder-free medium. BU3 NGPT iPSCs were generated and maintained in house with an Institutional Review Board approval. KOLF2-C1 clone D05 iPSC (46XY, normal karyotype by G-banding) were obtained (THE JACKSON LABORATORY FOR GENOMIC MEDICINE).

Briefly, cryopreserved iBCs were thawed and cultured in 3D-MATRIGEL droplets in PNEUMACULT EX-PLUS media (STEMCELL TECHNOLOGIES, 5040) supplemented with A83-01, DMH-1, Y27632 and PRIMOCIN. Seven days after thawing, the 3D iBC cultures were dissociated in dispase and trypsin as described for mouse iBC cultures, and were transduced with pHAGE-EF1α_{L}-tdTomato-W lentivirus at MOI of 10 for three hours in suspension, before replating in 3D-MATRIGEL at 400 cells/μL. After 10-14 days of culture, NGFR+/tdTomato+ were purified by flow cytometry and plated again in 3D-MATRIGEL at 400 cells/μL. After 14 days of further culture, the iBCs were harvested for transplantation without further cell sorting.

### Reverse Transcription Quantitative PCR (RT-qPCR)

RNA was extracted by first lysing cells in QIAZOL (QIAGEN, 79306) or RLT Plus lysis buffer (QIAGEN, 1053393), and subsequently using the RNEASY MINI kit (QIAGEN, 74104). TAQMAN FAST UNIVERSAL PCR Master Mix (APPLIED BIOSYSTEMS, 4364103) was used to reverse transcribe RNA into cDNA, followed by 40 cycles of real time quantitative PCR (qPCR) using Taqman probes (see e.g., Table 1). Relative gene expression, normalized to 18S control, was calculated as fold change in 18S-normalized gene expression, over baseline, using the 2^{-ΔΔCT} method. Baseline, defined as fold change=1, was set to undifferentiated PSCs or primary control as indicated in the text. If undetected, a cycle number of 40 was assigned to allow fold change calculations.

### Transplantation of BC iBC Culture

Adult recipient mice (8-16 weeks old) were used for all transplantation studies. Tracheal epithelial injury was induced in isoflurane anesthetized mice by intratracheal delivery of 20 μL of 2% polidocanol using the tongue pull method, followed by recovery prior to cell delivery 5-5.5 hours later. Specifically, the recipient animal was anesthetized with 3% isoflurane until the animal developed exaggerated respiratory movements at rates of 1-1.5 breath/second. Then the mouse was lightly restrained by operator, while a second operator delivered polidocanol solution directly into the posterior oropharynx of the mouse. The nose was gently held to encourage the animal to aspirate through the mouse, about 2-4 times, or until the liquid was no longer visible. For cell transplants, mice were re-anesthetized and 6×10⁶ primary BCs or iBCs suspended in 20 μL of DMEM were intratracheally instilled as above.

### Mouse Tissue Harvest and Whole Mount Fluorescence Imaging

Recipient mice were sacrificed following isoflurane overdose and PBS perfusion through the right ventricle. Mouse tracheas were harvested from larynx to carina and opened by longitudinal transection of the anterior trachea through the cartilaginous rings. For whole mount imaging, the opened trachea was placed on a glass slide and imaged by fluorescence microscopy using a NIKON ECLIPSE NI-E microscope. Epifluorescence imaging quantification was performed with the IMAGE J (Version 2.1.0/1.53i). Specifically, the “Color Threshold” function was used to select and calculate the area of GFP/RFP+ regions (based on GFP/RFP signal) as well as the entire recipient trachea (based on background autofluorescence). Transplantation efficiency was calculated by dividing the GFP/RFP+ area over total trachea area. To prepare 7 m thick tissue sections for immunostaining, the tracheal tissue was then fixed with 4% PFA at 4° C. for 4 hours prior to embedding in paraffin.

### Immunofluorescence Microscopy

Fixed, paraffin-embedded mouse trachea or lung 7 m thick tissue sections were rehydrated using standard methods and treated with citric acid-based Antigen Unmasking Solution (VECTOR LABORATORIES, H-3300-250) according to the manufacturer's instructions. Slides were washed in PBS and blocking with 10% Normal donkey serum (SIGMA, D9663), 2% Bovine Serum Albumin (FISHER SCIENTIFIC, BP1600), and 0.5% TRITON X-100 (Sigma, T9284, octyl phenol ethoxylate) for 1 hr at room temperature. The M.O.M. blocking kit (VECTOR LABORATORIES, BMK-2202) was used when primary mouse antibodies were required. Sections were incubated in primary antibody (see e.g., Table 1) resuspended in blocking buffer diluted 1:4 in PBS overnight at 4° C., washed 3× with PBS, and incubated in secondary antibody (see e.g., Table 1) and Hoechst 33342 (1:1000, THERMOFISHER) for 2 hr at room temperature. Slides were subsequently washed 1× with PBS and mounted using FLUOROSAVE mounting reagent (MILLIPORE, 345789). Slides were imaged using either NIKON ECLIPSE NI-E microscope or ZEISS LSM710 confocal microscope.

### Fluorescence-Activated Cell Sorting (FACS) or Analysis

Single cell suspensions were prepared as described for passaging (in vitro samples) or lung digestion. When indicated, cells were stained with fluorescence-conjugated antibodies (see e.g., Table 1) for 30 minutes in FACS buffer (2% Fetal Bovine Serum in PBS) at 4° C. and then resuspended in FACS buffer with 1:100 DRAQ7 or 1:1000 Calcein Blue, AM (THERMOFISHER, C1429) (live/dead stain). FACS was performed on either a BECKMAN COULTER MOFLO ASTRIOS or BD FACSARIA II SORP. Flow analysis was performed on a BECKMAN COULTER MOFLO ASTRIOS or STRATEDIGM S1000EXi. Data analysis was performed with FLOWJO software (version 10.8.1).

### Serial Secondary Transplantation

GFP+ cells were harvested from transplant recipients 6-16 weeks post-transplantation, using the same method to harvest primary basal cells as described above. GFP+ cells were then isolated by FACS and cultured in BCM. To achieve the cell number necessary for the next round of transplantation, passage 0 outgrowth of GFP+ cells were passaged and purified on Nkx2-1^{mCherry}+/GFP+/NGFR+FACS gating after 10-14 days of culture in BCM. Then, the passage 1 outgrowth cells were transplanted into syngeneic recipients as described above.

### Single Cell RNA Sequencing

Single cell suspensions were prepared, and live epithelial cells were FACS purified on a BECKMAN COULTER MOFLO ASTRIOS cell sorter as described above to collect populations described in the results section. Single-cell RNA-sequencing was performed using the CHROMIUM SINGLE CELL 3′ system (10× GENOMICS) at q Single Cell Sequencing Core according to the manufacturer's instructions (10× GENOMICS). Fastq files were generated using bcl2fastq v2.2 and CELLRANGER v.3.0.2. The sequenced files were mapped to the GRCm38/mm10 and/or GRCh30/hg38 supplemented with GFP, mCherry and tdTomato transcripts. Seurat v.3 was used to process the data further. Doublet rate was estimated according to the 10× chromium guidelines, in proportion to the density of cells loaded. These rates helped to flag potential doublets based on their gene and UMO counts. Based on manufacturer's recommendations for sequencing an average of >40,000 reads per cell to detect 1000-3000 genes per cell, sequencing depth was pursued to achieve these guidelines for all mouse cells, and dead cells or outliers were excluded by excluding any cells with fewer than 200 genes detected, as well as any cells with higher percentages of counts mapping to mitochondrial genes by setting a threshold of 15%, according to recommendation from the Seurat Package. For the human cell recipients, endogenous mouse cells were initially included in the sequencing experiment to generate cell numbers necessary for the technical processing of the sample. Therefore, these samples were mapped onto both GRCm38/mm10 and GRCh30/hg38. Post alignment, the Seurat object was created for both species. These objects were merged and then normalized using SCTransform, with cell degradation effect regressed out. After an initial linear dimensionality reduction (PCA), UMAP projections were used to represent the data, and the Louvain algorithm was used for clustering without harmonization. Differential gene expression tests were done with MAST, with prior gene filters to reduce the burden of multiple test corrects (minimum gene detection percentage of 10% in at least one of the populations, minimum average log-fold change of 0.5 between the two populations). The data was then visualized using SPRING plot. In the case of in vivo samples, Louvain clustering was used to identify and remove non-epithelial cells. Samples from distinct runs were then combined without harmonization, and again clustered using Louvain clustering. For merged dataset analysis, Seurat objects of individual datasets were merged together without harmonization and analyzed.

### In Vivo Bioluminescence Imaging

The XENOGEN IVIS Spectrum Instrument and Living Image Software Version 3.2 (CALIPER LIFESCIENCES) were used for imaging, analysis, and quantification of bioluminescence signals. The fur on the ventral side of the mice were removed by NAIR prior to imaging. On the day of imaging, luciferin (PALMER ELMER, 122799, 15 mg/kg body weight) was administered via intra-peritoneal injection. Animals were imaged immediately after the injection.

### Cilia Imaging

Mice were euthanized, perfused, and the trachea was harvested as described. The apical luminal surfaces of the lung epithelia were labelled using a solution of PBS pH 7.4 supplemented with 1 μg/ml of ALEXA FLUOR 647 conjugated Wheat Germ Agglutinin (THERMOFISHER, W32466) for 10 mins. This procedure was performed by insertion of a flexible cannula into the trachea which was held in place with a surgical suture, and the lungs were inflated using a reservoir held at 35 cm above the surgical site. The trachea was removed by the release of the suture and further dissection above the branch point and butterflied across the tracheal rings and washed 3 times with PBS pH 7.4 before proceeding to imaging.

The labelled trachea was then placed with the luminal surface facing down between two glass slips held apart by glass spacers at 0.85 mm in height. Motile cilia were imaged using a ZEISS LSM880 confocal microscope using the FAST AIRYSCAN mode in line sequential mode using a PLAN-APOCHROMAT 20X/0.8 M27 objective at 1 frame per sec. Whole mounted imaging was performed by tilling in Z-stack mode using AIRYSCAN mode in line sequential mode using a PLAN-APOCHROMAT 20X/0.8 M27 objective. The raw data was processed with ZEN BLACK. Time differential analysis and kymograph analysis was performed on acquired videos. Cilia length was calculated by measuring individual cilia while in orthogonal view of the 3D acquired images. Tissue sections were scanned at 20× using a ZEISS AXIO SCAN.Z1 slide scanner.

### Quantification and Statistical Analysis

Statistical methods relevant to RT-qPCR, immunostaining, or flow cytometry assessment are outlined in the figure legends. Paired, two-tailed Student's t tests were used for comparisons involving only two groups, while one-way ANOVA was used when considering multiple groups. Statistical analysis was done in GRAPHPAD PRISM, and p value<0.05 was used to determine statistical significance unless otherwise indicated in the text.

### Resources

TABLE 1
Key resources table

| | | |
|---|---|---|
| REAGENT or RESOURCE | SOURCE | IDENTIFIER |

Antibodies

| | | |
|---|---|---|
| Rabbit Anti-NGFR Antibody (no longer in | ABCAM | Cat#ab8875 |
| production) | | |
| Mouse Anti-NGFR Antibody | ABCAM | Cat#ab245134 |
| BV421 Rat Anti-Mouse CD326 | BD BIOSCIENCES | Cat#563214 |
| Goat Anti-GFP Antibody | US BIOLOGICAL | Cat#G8965-01E |
| Chicken Anti-GFP Antibody | AVES LABS | Cat#GFP-1020 |
| Goat Anti-RFP Antibody | MY BIOSOURCE | Cat#MBS448122 |
| Rat Anti-Mki67 Antibody | THERMO FISHER | Cat#14-5698-82 |
| Rabbit Anti-NKX2-1 Antibody | ABCAM | Cat#ab76013 |
| Rabbit Anti-KRT5 Antibody | BIOLEGEND | Cat#905501 |
| Rabbit Anti-RFP Antibody | ROCKLAND | Cat#600-401-379 |
| Mouse Anti-Acetylated-Tubulin Antibody | SIGMA | Cat#T7451 |
| Goat Anti-SCGB1A1 (mouse) Antibody | Gift | |
| Goat Anti-SCGB1A1 (human) Antibody | SIGMA | Cat#ABS1673 |
| Rabbit Anti-smooth muscle actin Antibody | ABCAM | Cat#ab5694 |
| Rat Anti-E-Cadherin Antibody | INVITROGEN | Cat#13-1900 |
| Goat Anti-CD45 Antibody | R&D SYSTEMS | Cat#AF114 |
| ALEXA FLUOR 488 donkey anti-chicken IgY | JACKSON | Cat#703-545-155 |
| (IgG) (H + L) | IMMUNO | |
| | RESEARCH | |
| ALEXA FLUOR 488 donkey anti-goat IgG (H + L) | JACKSON | Cat#705-545-003 |
| | IMMUNO | |
| | RESEARCH | |
| ALEXA FLUOR 488 donkey anti-rat IgG (H + L) | JACKSON | Cat#712-546-153 |
| | IMMUNO | |
| | RESEARCH | |
| ALEXA FLUOR 488 donkey anti-rabbit IgG (H + L) | JACKSON | Cat#711-546-152 |
| | IMMUNO | |
| | RESEARCH | |
| ALEXA FLUOR 488 donkey anti-mouse IgG | JACKSON | Cat#705-545-150 |
| (H + L) | IMMUNO | |
| | RESEARCH | |
| ALEXA FLUOR 546 donkey anti-Rabbit IgG | THERMO FISHER | Cat#A11055 |
| (H + L) | | |
| ALEXA FLUOR 568 donkey anti-mouse IgG | THERMO FISHER | Cat#A10037 |
| (H + L) | | |
| Cy3 donkey anti-rat IgG (H + L) | JACKSON | Cat#712-166-153 |
| | IMMUNO | |
| | RESEARCH | |
| Cy3 donkey anti-rabbit IgG (H + L) | JACKSON | Cat#711-165-152 |
| | IMMUNO | |
| | RESEARCH | |
| Cy3 donkey anti-goat IgG (H + L) | JACKSON | Cat#705-165-147 |
| | IMMUNO | |
| | RESEARCH | |
| ALEXA FLUOR 647 donkey anti-rabbit IgG (H + L) | THERMO FISHER | Cat#A32795 |
| ALEXA FLUOR 647 donkey anti-goat IgG (H + L) | JACKSON | Cat#705-605-003 |
| | IMMUNO | |
| | RESEARCH | |

Bacterial and virus strains

| | | |
|---|---|---|
| pHAGE-EF1_{αL}-GFP-W lentivirus | Addgene | ADDGENE126686 |
| pHAGE-EF1_{αL}-tdTomato-W lentivirus | in house | |
| CMV-Luciferase-EF1_{α}-copGFP lentivirus | SYSTEM | Cat#BLIV511PA-1 |
| | BIOSCIENCES | |

Biological samples

| | | |
|---|---|---|
| Primary human airway epithelial cell culture | a Tissue | N/A |
| | Procurement and | |
| | Cell Culture Core | |

Chemicals, peptides, and recombinant proteins

| | | |
|---|---|---|
| DMEM | GIBCO | Cat#2414671 |
| IMDM | THERMOFISHER | Cat#12440053 |
| GLUTAMAX | LIFE | Cat#35050-061 |
| | TECHNOLOGIES | |
| Ham's F12 | CELLGRO | Cat#10-080-CV |
| B27 supplement | INVITROGEN | Cat#17504-44 |
| N2 Supplement | INVITROGEN | Cat#17502-048 |
| PRIMOCIN | INVIVOGEN | Cat#ANTPM1 |
| 7.5% BSA Fraction V | THERMOFISHER | Cat#15260-037 |
| 1-thioglycerol (MTG) | SIGMA | Cat#M6145 |
| Ascorbic Acid | SIGMA | Cat#A4544 |
| Recombinant human/mouse/rat Activin A | R&D SYSTEMS | Cat#338-AC |
| Recombinant mouse Wnt3a | R&D SYSTEMS | Cat#1324-WN-010 |
| Recombinant human Bmp4 | R&D SYSTEMS | Cat#314-BP |
| Recombinant mouse Noggin | R&D SYSTEMS | Cat#1967-NG |
| SB431542 | SIGMA | Cat#S4317 |
| Recombinant human FGF2 | R&D SYSTEMS | Cat#233-FB |
| Recombinant human FGF10 | R&D SYSTEMS | Cat#345-FG |
| DMH-1 | TOCRIS | Cat#4126 |
| A8301 | TOCRIS | Cat#2939 |
| Y-27632 (ROCK inhibitor) | TOCRIS | Cat#1254 |
| Heparin Salt | SIGMA | Cat#H3393 |
| | MILLIPORE | |
| Growth Factor Reduced 3D MATRIGEL | CORNING | Cat#354230 |
| Dispase | GIBCO | Cat#17105-041 |
| Collagenase Type IV | THERMOFISHER | Cat#17104019 |
| PNEUMACULT EXPLUS Medium | STEM CELL | Cat#5040 |
| | TECHNOLOGIES | |
| BEGM | a Tissue | N/A |
| | Procurement and | |
| | Cell Culture Core | |
| Retinoic acid | SIGMA | Cat#R2625 |
| 0.05% Trypsin-EDTA | GIBCO | Cat#25300062 |
| fetal bovine serum | GIBCO | Cat#16141079 |
| PURECOL | ADVANCED | Cat#5005 |
| | BIOMATRIX | |
| Paraformaldehyde | ELECTRON | Cat#19208 |
| | MICROSCOPY | |
| | SCIENCES | |
| Normal Donkey Serum | SIGMA | Cat#D9663 |
| Antigen Unmasking Solution, Citric Acid Based | VECTOR | Cat#H-3300-250 |
| | LABORATORIES | |
| M.O.M. Immunodetection Kit | VECTOR | Cat#BMK-2202 |
| | LABORATORIES | |
| Bovine Serum Albumin | FISHER | Cat#BP1600 |
| FLUOROSAVE Mounting Agent | MILLIPORE | Cat#345789 |
| Hoechst 33342 | THERMO FISHER | Cat#H3570 |
| TRITON-X | SIGMA | Cat#T9284 |
| Pronase | ROCHE | Cat#10165921001 |
| Wheat Germ Agglutinin, Alexa Fluor ™ 647 | THERMOFISHER | Cat#W32466 |
| Conjugate | | |
| DNAse | ROCHE | Cat#10104159001 |
| Polidocanol | SIGMA | Cat#88315 |
| XENOLIGHT D-Luciferin | PERKINELMER | Cat#122799 |
| Calcein Blue | THERMOFISHER | Cat#C1429 |
| DRAQ7 | ABCAM | Cat#ab109202 |

Critical commercial assays

| | | |
|---|---|---|
| RNEASY MINI Kit | QIAGEN | Cat#74104 |
| QIAZOL Lysis Reagent QIAGEN | QIAGEN | Cat#79306 |
| RLT Plus lysis buffer | QIAGEN | Cat#1053393 |
| TAQMAN FAST UNIVERSAL PCR Master Mix | THERMO FISHER | Cat#4364103 |
| (2X) | | |
| High-Capacity cDNA Reverse Transcription Kit | APPLIED | Cat#4368814 |
| | BIOSYSTEMS | |
| Click-iT EdU Cell Proliferation Kit | THERMOFISHER | Cat#C10340 |

Deposited data

| | | |
|---|---|---|
| Sequence Data | described herein | GEO: Super Series |
| | | GSE211998 |

Experimental models: Cell lines

| | | |
|---|---|---|
| Nkx2-1^{mCherry} mouse ES Cell Line | Bilodeau et al. Stem | N/A |
| | Cell Rep 3, 634- | |
| | 649, 2014 | |
| Human BU3-NGPT iPSC Cell Line | in house | N/A |
| Human KOLF2 iPSC Cell Line | Skarnes et al. | N/A |
| | Methods 164, 18- | |
| | 28, 2019 | |

Experimental models: Organisms/strains

| | | |
|---|---|---|
| UBC-GFP mice | JACKSON LABS | JAX004353 |
| C57BL/6J mice | JACKSON LABS | JAX000664 |
| 129X1/SvJ mice | JACKSON LABS | JAX000691 |
| 129S1/SvlmJ mice | JACKSON LABS | JAX002448 |
| NSG mice | JACKSON LABS | JAX005557 |

Software and algorithms

| | | |
|---|---|---|
| FLOWJO Software v.10.8.1 | BECTON | flowjo.com/solutions/flowjo |
| | DICKINSON & | |
| | COMPANY | |
| Seurat v.3 | | github.com/satijalab/seurat |
| SPRING | | github.com/AllonKleinLab/SPRINGdev |
| LIVING IMAGE Software v. 3.2 | CALIPER | perkinelmer.com/product/spectrum-200- |
| | LIFESCIENCES | living-image-v4series-1-128113 |
| GRAPHPAD PRISM v.9.5.1 | GRAPHPAD | graphpad.com/features |
| | SOFTWARE | |
| IMAGEJ v.2.1.0/1.53i | NIH | imagej.net/ij/index.html |

Other

| | | |
|---|---|---|
| Taqman Probe: Nkx2-1 | THERMO FISHER | Mm00447558_ml |
| Taqman Probe: Trp63 | THERMO FISHER | Mm00495788_ml |
| Taqman Probe: Krt5 | THERMO FISHER | Mm01305291_gl |
| Taqman Probe: Ngfr | THERMO FISHER | Mm00446296_ml |
| Taqman Probe: Scgb1a1 | THERMO FISHER | Mm00442046_ml |
| Taqman Probe: Muc5ac | THERMO FISHER | Mm01276718_ml |
| Taqman Probe: Foxj1 | THERMO FISHER | Mm01267279_ml |

What is claimed herein is:

1. A method of engrafting airway basal cells into the respiratory tract of a subject in need thereof, the method comprising:
(a) removing epithelial cells from the respiratory tract; and
(b) administering airway basal cells to the respiratory tract.

2. The method of claim 1, wherein the epithelium cells are removed from the respiratory tract a chemical agent or using bronchial thermoplasty.

3. The method of claim 2, wherein the chemical agent comprises a detergent.

4. The method of claim 2, wherein the chemical agent is selected from the group consisting of: polidocanol; 3-[(3-cholamidopropyl)dimethylammonio]-1-propane sulfonate (CHAPS); lucinactant, colfosceril palmitate, beractant, calefacient, and poractant alfa.

5. The method of claim 2, wherein the chemical agent comprises polidocanol.

6. The method of claim 1, wherein step (b) of administering the airway basal cells occurs 3 hours to 24 hours after step (a) of removing epithelium epithelial cells from of the respiratory tract.

7. The method of claim 1, wherein the epithelial cells are removed in the trachea, at least one bronchus, and/or at least one bronchiole of the respiratory tract.

8. The method of claim 1, wherein the airway basal cells are administered to the trachea, at least one bronchus, and/or at least one bronchiole of the respiratory tract.

9. The method of claim 1, wherein the chemical agent and/or the airway basal cells are administered using a bronchoscope.

10. The method of claim 1, wherein the airway basal cells are primary cells.

11. The method of claim 1, wherein the airway basal cells are differentiated from pluripotent stem cells.

12. The method of claim 1, wherein the airway basal cells express at least one marker selected from the group consisting of: Tumor Protein P63 (TRP63), keratin-5 (KRT5), and NK2 Homeobox 1 (NKX2-1).

13. The method of claim 1, wherein the airway basal cells are autologous or allogeneic to the subject.

14. The method of claim 1, wherein the airway basal cells are engineered to reduce or prevent expression of at least one major histocompatibility complex (MHC) polypeptide to reduce immunogenicity of the airway basal cells.

15. The method of claim 1, wherein the subject is healthy.

16. The method of claim 1, wherein the subject has a respiratory tract disease or a respiratory tract injury.

17. The method of claim 16, wherein the respiratory tract disease is selected from the group consisting of: cystic fibrosis; primary ciliary dyskinesia; idiopathic pulmonary fibrosis (IPF); chronic obstructive pulmonary disease (COPD); lung cancer; asthma; chronic bronchitis; bronchiectasis; bronchiolitis obliterans syndrome; and chronic lung allograft disorder (CLAD).

18. The method of claim 16, wherein the respiratory tract injury is associated with: inhalation of a chemical (e.g., an acid), smoke, fire, and/or a heated substance; a burn; radiation damage; chemotherapy damage; space travel; a respiratory infection; a birth defect; a lung cancer; and/or a surgery (e.g., tracheal resection, lung lobectomy).

19. The method of claim 1, wherein the airway basal cells are engineered to express at least one functional polypeptide that is dysfunctional in a respiratory tract disease.

20. The method of claim 19, wherein the airway basal cells are engineered to express functional Cystic Fibrosis Transmembrane Conductance Regulator (CFTR) polypeptide, and the subject has cystic fibrosis.

21. The method of claim 19, wherein the airway basal cells are engineered to express functional Dynein Axonemal Heavy Chain 5 (DNAH5) polypeptide and/or functional Dynein Axonemal Intermediate Chain 1 (DNAI1) polypeptide, and the subject has primary ciliary dyskinesia.

22. The method of claim 1, wherein the engrafted airway basal cells reconstitute the tissue-resident airway basal stem cell compartment in the respiratory tract of the subject.

23. The method of claim 1, wherein the engrafted airway basal cells comprise at least 10% of the epithelium in the respiratory tract of the subject after the engraftment.

24. The method of claim 1, wherein the engrafted airway basal cells persist for at least 2 years following the engraftment.

25. The method of claim 1, wherein the engrafted airway basal cells exhibit self-renewal and/or multipotent differentiation capacity for at least 2 years following the engraftment.

26. The method of claim 25, wherein the multipotent differentiation capacity comprises: basal cell, secretory cell, ciliated cell, ionocyte, neuroendocrine cell, tuft cell, and/or hillock cell airway epithelial lineages.

27. The method of claim 1, wherein the engrafted airway basal cells exhibit a transcriptome that is at least 80% identical to the transcriptome of endogenous airway epithelium cells.

28. A method of treating a respiratory tract disease, the method comprising:
(a) removing epithelial cells from the respiratory tract of a subject in need thereof, and
(b) administering an effective amount of airway basal cells to the respiratory tract;
wherein the respiratory tract disease is selected from the group consisting of: cystic fibrosis; primary ciliary dyskinesia; idiopathic pulmonary fibrosis (IPF); chronic obstructive pulmonary disease (COPD); lung cancer; asthma; chronic bronchitis; bronchiectasis; bronchiolitis obliterans syndrome; and chronic lung allograft disorder (CLAD).

29. A method of treating a respiratory tract injury, the method comprising:
(a) removing epithelial cells from the respiratory tract of a subject in need thereof, and
(b) administering an effective amount of airway basal cells to the respiratory tract;
wherein the respiratory tract injury is associated with: inhalation of a chemical (e.g., an acid), smoke, fire, and/or a heated substance; a burn; radiation damage; chemotherapy damage; space travel; a respiratory infection; a birth defect; a lung cancer; and/or a surgery (e.g., tracheal resection, lung lobectomy).

30. A kit comprising:
(a) a chemical agent to remove epithelial cells from a respiratory tract; and
(b) airway basal cells.
